(19) United States
(12) Patent Application Publication
(10) Pub. No.: US 2025/0262327 A1
(43) Pub. Date: Aug. 21, 2025

(54) COMPOSITIONS AND METHODS FOR TREATING DOMINANT OPTIC ATROPHY AND X-LINKED RETINOSCHISIS

(71) Applicant: Abeona Therapeutics Inc., Cleveland, OH (US)

(72) Inventor: Linas PADEGIMAS, Mayfield, OH (US)

(21) Appl. No.: 18/857,323

(22) PCT Filed: Apr. 18, 2023
(86) PCT No.: PCT/US2023/065877

§ 371 Date: Oct. 16, 2024

Related U.S. Application Data
(60) Provisional application No. 63/332,015, filed on Apr. 18, 2022.

(51) Int. Cl. A61K 48/00; C12N 15/86

(52) U.S. Cl. CPC A61K 48/0058; C12N 15/86; C12N 2750/14143

(57) ABSTRACT

Provided herein are recombinant AAV vectors, AAV viral vectors, capsid proteins, and administration methods for improved gene therapy, and methods for their manufacture and use. These AAV vectors may be used for treating retinoschisis (e.g., X-linked Retinoschisis) or Dominant Optic Atrophy (DOA).

### CROSS-REFERENCE TO RELATED APPLICATIONS

This application claims the benefit of U.S. Provisional Application No. 63/332,015, filed Apr. 18, 2022, the content of which is incorporated by reference in its entirety.

### REFERENCE TO AN ELECTRONIC SEQUENCE LISTING

The content of the electronic sequence listing (ABEO_009_01WO_SeqList_ST26.xml; Size: 732,965 bytes; and Date of Creation: Apr. 13, 2023) is herein incorporated by reference in its entirety.

### BACKGROUND

X-linked retinoschisis (XLRS) is a rare, monogenic disease that results in severe visual impairment. While female carriers are asymptomatic, affected males usually begin exhibiting disease symptoms within the first decade, and occasionally during infancy. The disease results from mutations in the RS1 gene, which is expressed in photoreceptors and retinal bipolar cells. In individuals with XLRS, cavities develop where the adhesion of adjacent retinal layers is disrupted, leading to discontinuity within the retinal circuitry, photoreceptor degeneration, and impaired visual acuity. The current standard of care for XLRS patients is palliative and involves correction of refractive errors, low-vision aids, and genetic counseling. Complications such as retinal detachment (up to 22% of patients) and vitreal hemorrhage (up to 40% of patients) are most frequent in the later stages of disease and can be treated surgically. Early intervention via gene therapy has significant potential to reverse or stabilize disease progression at early stages of disease and prevent significant vision loss as well as the occurrence of these more severe complications.

Autosomal Dominant Optic Atrophy (ADOA) results in vision loss in the second to third decade of life. ADOA subjects typically have retinal degeneration, neurological defects, and musculoskeletal complications over time. ADOA caused by Opa1 mutation(s). Opa1 has been shown to be involved in mitochondrial christae structure, mitochondrial fusion, and mitochondrial inner membrane remodeling.

There is a need for new gene therapies for treating XLRS or ADOA.

### SUMMARY

The present disclosure relates generally to the field of gene therapy and in particular, to recombinant adeno-associated viral (AAV) vector particles (also known as AAV viral vectors) with novel expression cassettes, and their use to deliver transgenes to treat or prevent a disease or disorder such as XLRS or ADOA.

In one aspect, the disclosure provides methods of treating retinoschisis in a subject in need thereof, comprising para-retinal or sub-retinal administration of an AAV viral vector to the subject. In embodiments, the AAV viral vector comprises a photoreceptor-specific promoter operably linked to a transgene encoded by a heterologous nucleic acid. In embodiments, the photoreceptor-specific promoter is selected from the group consisting of a rhodopsin kinase (RK) promoter, a rhodopsin (RHO) promoter, a beta phosphodiesterase (PDE) promoter, and a retinitis pigmentosa (RP1) promoter. In embodiments, the photoreceptor-specific promoter is a rhodopsin kinase (RK) promoter. In embodiments, the RK promoter comprises, consists essentially of, or consists of a nucleic acid sequence having at least 90%, at least 95%, at least 97%, at least 98%, at least 99%, or 100% identity to SEQ ID NO: 196. In embodiments, the method comprises para-retinal administration of the AAV viral vector to the subject. In embodiments, the subject is a human. In embodiments, the AAV viral vector is administered at a dose of about 10¹⁰ to about 1012 viral genome (vg). In embodiments, the retinoschisis is X-linked retinoschisis. In embodiments, the transgene is RS1. In embodiments, the transgene comprises a nucleic acid sequence having at least 90%, at least 95%, at least 97%, at least 98%, at least 99%, or 100% identity to SEQ ID NO: 117. In embodiments, the transgene encodes an amino acid sequence having at least 90%, at least 95%, at least 97%, at least 98%, at least 99%, or 100% identity to SEQ ID NO: 143.

In one aspect, the disclosure provides methods of treating an ocular disease or disorder in a subject in need thereof, comprising administration of an AAV viral vector to the subject, wherein the AAV viral vector comprises an AAV vector genome, wherein the AAV vector genome comprises, in 5′ to 3′ orientation: (a) a first AAV inverted terminal repeat, (b) a promoter, (c) a heterologous nucleic acid encoding Opa1, (d) a polyadenylation signal, and (e) a second AAV inverted terminal repeat.

In embodiments, the promoter is a CBh promoter comprising a sequence having at least 90%, at least 95%, at least 97%, at least 98%, at least 99%, or 100% identity to SEQ ID NO: 154. In embodiments, the promoter is a MeCP2 promoter comprising a sequence having at least 90%, at least 95%, at least 97%, at least 98%, at least 99%, or 100% identity to SEQ ID NO: 156.

In embodiments, the AAV vector genome comprises an intron sequence having at least 90%, at least 95%, at least 97%, at least 98%, at least 99%, or 100% identity to SEQ ID NO: 200 or 227. In embodiments, the intron sequence is located between the promoter and the heterologous nucleic acid encoding Opa1. In embodiments, the intron sequence is located immediately downstream of the promoter, without any additional nucleotide in between.

In embodiments, the heterologous nucleic acid encoding Opa1 comprises a sequence having at least 90%, at least 95%, at least 97%, at least 98%, at least 99%, or 100% identity to any one of SEQ ID NO: 175, 182 and 184. In embodiments, the heterologous nucleic acid encodes an Opa1 protein comprising an amino acid sequence having at least 90%, at least 95%, at least 97%, at least 98%, at least 99%, or 100% identity to any one of SEQ ID NO: 180, 183 and 185. In embodiments, the heterologous nucleic acid encodes an Opa1 protein comprising an amino acid sequence having at least 90%, at least 95%, at least 97%, at least 98%, at least 99%, or 100% identity to SEQ ID NO: 180.

In embodiments, the polyadenylation signal comprises a sequence having at least 90%, at least 95%, at least 97%, at least 98%, at least 99%, or 100% identity to SEQ ID NO: 201 or 225.

In embodiments, the AAV vector genome does not comprise any telomeric repeats sequence.

In embodiments, the AAV vector genome comprises a first telomeric repeats sequence located between the polyadenylation signal and the second AAV inverted terminal repeat. In embodiments, the first telomeric repeats sequence has at least 90%, at least 95%, at least 97%, at least 98%, at least 99%, or 100% identity to SEQ ID NO: 202. In embodiments, the AAV vector genome comprises a second telomeric repeats sequence located between the first AAV inverted terminal repeat and the promoter. In embodiments, the second telomeric repeats sequence has at least 90%, at least 95%, at least 97%, at least 98%, at least 99%, or 100% identity to SEQ ID NO: 203.

In embodiments, the first AAV inverted terminal repeat comprises or consists of a sequence having at least 90%, at least 95%, at least 97%, at least 98%, at least 99%, or 100% identity to SEQ ID NO: 253. In embodiments, the second AAV inverted terminal repeat comprises or consists of a sequence having at least 90%, at least 95%, at least 97%, at least 98%, at least 99%, or 100% identity to SEQ ID NO: 254.

In embodiments, the AAV vector genome comprises, in 5′ to 3′ orientation: (a) the first AAV inverted terminal repeat, (b) the promoter comprising a sequence having at least 90%, at least 95%, at least 97%, at least 98%, at least 99%, or 100% identity to SEQ ID NO: 154, (c) the heterologous nucleic acid encoding Opa1, (d) the polyadenylation signal comprises a sequence having at least 90%, at least 95%, at least 97%, at least 98%, at least 99%, or 100% identity to SEQ ID NO: 201, and (e) the second AAV inverted terminal repeat. In embodiments, the AAV vector genome comprises an intron sequence having at least 90%, at least 95%, at least 97%, at least 98%, at least 99%, or 100% identity to SEQ ID NO: 200. In embodiments, the intron sequence is located between the promoter and the heterologous nucleic acid encoding Opa1. In embodiments, the AAV vector genome comprises a sequence having at least 90%, at least 95%, at least 97%, at least 98%, at least 99%, or 100% identity to SEQ ID NO: 228. In embodiments, the Opa1 protein comprises an amino acid sequence having at least 90%, at least 95%, at least 97%, at least 98%, at least 99%, or 100% identity to SEQ ID NO: 180.

In embodiments, the AAV vector genome comprises a polynucleotide sequence having at least 90%, at least 95%, at least 97%, at least 98%, at least 99%, or 100% identity to any one of SEQ ID NO: 230-239.

In embodiments, the ocular disease or disorder is Autosomal Dominant Optic Atrophy.

In one aspect, the disclosure provides methods of treating an ocular disease or disorder in a subject in need thereof, comprising administration of an AAV viral vector to the subject, wherein the AAV viral vector comprises an AAV vector genome, wherein the AAV vector genome comprises, in 5′ to 3′ orientation: (a) a first AAV inverted terminal repeat, (b) a promoter, (c) a heterologous nucleic acid encoding RS1, (d) a polyadenylation signal, and (e) a second AAV inverted terminal repeat.

In embodiments, the promoter is a photoreceptor-specific promoter. In embodiments, the photoreceptor-specific promoter is selected from the group consisting of a rhodopsin kinase (RK) promoter, a rhodopsin (Rho) promoter, a beta phosphodiesterase (PDE) promoter, and a retinitis pigmentosa (RP1) promoter. In embodiments, the promoter is a RK promoter comprising a sequence having at least 90%, at least 95%, at least 97%, at least 98%, at least 99%, or 100% identity to SEQ ID NO: 196. In embodiments, the promoter is a Rho promoter comprising a sequence having at least 90%, at least 95%, at least 97%, at least 98%, at least 99%, or 100% identity to SEQ ID NO: 197. In embodiments, the promoter is a PDE promoter comprising a sequence having at least 90%, at least 95%, at least 97%, at least 98%, at least 99%, or 100% identity to SEQ ID NO: 198.

In embodiments, the promoter is a CBh promoter comprising a sequence having at least 90%, at least 95%, at least 97%, at least 98%, at least 99%, or 100% identity to SEQ ID NO: 154.

In embodiments, the AAV vector genome comprises an IRBP enhancer sequence upstream of the promoter. In embodiments, the IRBP enhancer sequence has at least 90%, at least 95%, at least 97%, at least 98%, at least 99%, or 100% identity to SEQ ID NO: 199. In embodiments, the IRBP enhancer sequence is located immediately upstream of the promoter, without any additional nucleotide in between.

In embodiments, the AAV vector genome comprises a CVA-MVM intron sequence having at least 90%, at least 95%, at least 97%, at least 98%, at least 99%, or 100% identity to SEQ ID NO: 226. In embodiments, the CVA-MVM intron sequence is located between the promoter and the heterologous nucleic acid encoding RS1.

In embodiments, the AAV vector genome comprises an intron sequence having at least 90%, at least 95%, at least 97%, at least 98%, at least 99%, or 100% identity to SEQ ID NO: 200 or 222. In embodiments, the intron is located between the promoter and the heterologous nucleic acid encoding RS1.

In embodiments, the heterologous nucleic acid encoding RS1 comprises a sequence having at least 90%, at least 95%, at least 97%, at least 98%, at least 99%, or 100% identity to SEQ ID NO: 117. In embodiments, the heterologous nucleic acid encodes an RS1 protein comprising an amino acid sequence having at least 90%, at least 95%, at least 97%, at least 98%, at least 99%, or 100% identity to SEQ ID NO: 143.

In embodiments, the polyadenylation signal comprises a sequence having at least 90%, at least 95%, at least 97%, at least 98%, at least 99%, or 100% identity to SEQ ID NO: 201 or 225.

In embodiments, the AAV vector genome does not comprise any telomeric repeats sequence.

In embodiments, the AAV vector genome comprises a first telomeric repeats sequence located between the polyadenylation signal and the second AAV inverted terminal repeat. In embodiments, the first telomeric repeats sequence has at least 90%, at least 95%, at least 97%, at least 98%, at least 99%, or 100% identity to SEQ ID NO: 203.

In embodiments, the AAV vector genome comprises a human beta-globin scaffold/matrix attachment region (βGlo_s/MAR) sequence. In embodiments, the βGlo_s/MAR sequence is located between the polyadenylation signal and the second AAV inverted terminal repeat. In embodiments, the βGlo_s/MAR sequence is located between the polyadenylation signal and the first telomeric repeats sequence. In embodiments, the βGlo_s/MAR sequence has at least 90%, at least 95%, at least 97%, at least 98%, at least 99%, or 100% identity to SEQ ID NO: 221.

In embodiments, the first AAV inverted terminal repeat comprises or consists of a sequence having at least 90%, at least 95%, at least 97%, at least 98%, at least 99%, or 100% identity to SEQ ID NO: 255. In embodiments, the second AAV inverted terminal repeat comprises or consists of a sequence having at least 90%, at least 95%, at least 97%, at least 98%, at least 99%, or 100% identity to SEQ ID NO: 256.

In embodiments, the AAV vector genome comprises, in 5′ to 3′ orientation: (a) the first AAV inverted terminal repeat, (b) the IRBP enhancer sequence having at least 90%, at least 95%, at least 97%, at least 98%, at least 99%, or 100% identity to SEQ ID NO: 199, (c) the RK promoter comprising a sequence having at least 90%, at least 95%, at least 97%, at least 98%, at least 99%, or 100% identity to SEQ ID NO: 196, (d) the heterologous nucleic acid encoding RS1, (e) the polyadenylation signal comprising a sequence having at least 90%, at least 95%, at least 97%, at least 98%, at least 99%, or 100% identity to SEQ ID NO: 225, and (f) the second AAV inverted terminal repeat.

In embodiments, the AAV vector genome comprises an intron sequence having at least 90%, at least 95%, at least 97%, at least 98%, at least 99%, or 100% identity to SEQ ID NO: 222. In embodiments, the intron sequence is located between the promoter and the heterologous nucleic acid encoding RS1. In embodiments, the AAV vector genome comprises a CBA sequence of SEQ ID NO: 229, or a sequence having at most 5, at most 4, at most 3, at most 2, or at most 1 mutation(s) thereto. In embodiments, the CBA sequence is located immediately upstream of the intron sequence without any additional nucleotides in between. In embodiments, the AAV vector genome comprises a CBA-MVM intron sequence having at least 90%, at least 95%, at least 97%, at least 98%, at least 99%, or 100% identity to SEQ ID NO: 226. In embodiments, the CVA-MVM intron sequence is located between the promoter and the heterologous nucleic acid encoding RS1.

In embodiments, the AAV vector genome comprises a sequence having at least 90%, at least 95%, at least 97%, at least 98%, at least 99%, or 100% identity to SEQ ID NO: 224.

In embodiments, the AAV vector genome comprises a polynucleotide sequence having at least 90%, at least 95%, at least 97%, at least 98%, at least 99%, or 100% identity to any one of SEQ ID NO: 224 and 240-252.

In embodiments, the ocular disease or disorder is X-linked retinoschisis.

In embodiments, the AAV viral vector comprises an AAV capsid protein comprising an amino acid sequence that is at least 95% identical to SEQ ID NO: 1-3, 30-34, 49, 67, 84, or 164. In embodiments, the AAV viral vector comprises an AAV capsid protein comprising an amino acid sequence that is at least 98%, at least 99%, at least 99.5%, or 100% identical to SEQ ID NO: 1-3, 30-34, 49, 67, 84 or 164. In embodiments, the AAV viral vector comprises an AAV capsid protein comprising or consisting of an amino acid sequence that is at least 95%, at least 98%, at least 99%, at least 99.5%, or 100% identical to SEQ ID NO: 2.

In embodiments, the administration is para-retinal administration. In embodiments, the para-retinal administration comprises injecting at a distance of between 0 and 13 millimeters (mm), between 0 and 10 mm, between 0 and 5 mm, or between 0 and 3 mm, from the surface of the retina in the posterior vitreous cavity of the eye.

In embodiments, the subject is a human.

In one aspect, the disclosure provides nucleic acids comprising, in 5′ to 3′ orientation: (a) a first AAV inverted terminal repeat, (b) a promoter, (c) a heterologous nucleic acid encoding Opa1, (d) a polyadenylation signal, and (e) a second AAV inverted terminal repeat.

In embodiments, the promoter is a CBh promoter comprising a sequence having at least 90%, at least 95%, at least 97%, at least 98%, at least 99%, or 100% identity to SEQ ID NO: 154. In embodiments, the promoter is a MeCP2 promoter comprising a sequence having at least 90%, at least 95%, at least 97%, at least 98%, at least 99%, or 100% identity to SEQ ID NO: 156.

In embodiments, the AAV vector genome comprises an intron sequence having at least 90%, at least 95%, at least 97%, at least 98%, at least 99%, or 100% identity to SEQ ID NO: 200 or 227. In embodiments, the intron sequence is located between the promoter and the heterologous nucleic acid encoding Opa1. In embodiments, the intron sequence is located immediately downstream of the promoter, without any additional nucleotide in between.

In embodiments, the heterologous nucleic acid encoding Opa1 comprises a sequence having at least 90%, at least 95%, at least 97%, at least 98%, at least 99%, or 100% identity to any one of SEQ ID NO: 175, 182 and 184. In embodiments, the heterologous nucleic acid encodes an Opa1 protein comprising an amino acid sequence having at least 90%, at least 95%, at least 97%, at least 98%, at least 99%, or 100% identity to any one of SEQ ID NO: 180, 183 and 185. In embodiments, the heterologous nucleic acid encodes an Opa1 protein comprising an amino acid sequence having at least 90%, at least 95%, at least 97%, at least 98%, at least 99%, or 100% identity to SEQ ID NO: 180.

In embodiments, the polyadenylation signal comprises a sequence having at least 90%, at least 95%, at least 97%, at least 98%, at least 99%, or 100% identity to SEQ ID NO: 201 or 225.

In embodiments, the AAV vector genome does not comprise any telomeric repeats sequence.

In embodiments, the AAV vector genome comprises a first telomeric repeats sequence located between the polyadenylation signal and the second AAV inverted terminal repeat. In embodiments, the first telomeric repeats sequence has at least 90%, at least 95%, at least 97%, at least 98%, at least 99%, or 100% identity to SEQ ID NO: 202. In embodiments, the AAV vector genome comprises a second telomeric repeats sequence located between the first AAV inverted terminal repeat and the promoter. In embodiments, the second telomeric repeats sequence has at least 90%, at least 95%, at least 97%, at least 98%, at least 99%, or 100% identity to SEQ ID NO: 203.

In embodiments, the first AAV inverted terminal repeat comprises or consists of a sequence having at least 90%, at least 95%, at least 97%, at least 98%, at least 99%, or 100% identity to SEQ ID NO: 253. In embodiments, the second AAV inverted terminal repeat comprises or consists of a sequence having at least 90%, at least 95%, at least 97%, at least 98%, at least 99%, or 100% identity to SEQ ID NO: 254.

In embodiments, the AAV vector genome comprises, in 5′ to 3′ orientation: (a) the first AAV inverted terminal repeat, (b) the promoter comprising a sequence having at least 90%, at least 95%, at least 97%, at least 98%, at least 99%, or 100% identity to SEQ ID NO: 154, (c) the heterologous nucleic acid encoding Opa1, (d) the polyadenylation signal comprises a sequence having at least 90%, at least 95%, at least 97%, at least 98%, at least 99%, or 100% identity to SEQ ID NO: 201, and (e) the second AAV inverted terminal repeat.

In embodiments, the nucleic acid comprising an intron sequence having at least 90%, at least 95%, at least 97%, at least 98%, at least 99%, or 100% identity to SEQ ID NO: 200. In embodiments, the intron sequence is located between the promoter and the heterologous nucleic acid encoding Opa1.

In embodiments, the Opa1 protein comprises an amino acid sequence having at least 90%, at least 95%, at least 97%, at least 98%, at least 99%, or 100% identity to SEQ ID NO: 180.

In embodiments, the nucleic acid comprises a sequence having at least 90%, at least 95%, at least 97%, at least 98%, at least 99%, or 100% identity to SEQ ID NO: 228. In embodiments, the nucleic acid comprises a polynucleotide sequence having at least 90%, at least 95%, at least 97%, at least 98%, at least 99%, or 100% identity to any one of SEQ ID NO: 230-239.

In one aspect, the disclosure provides nucleic acids comprising, in 5′ to 3′ orientation: (a) a first AAV inverted terminal repeat, (b) a promoter, (c) a heterologous nucleic acid encoding RS1, (d) a polyadenylation signal, and (e) a second AAV inverted terminal repeat.

In embodiments, the promoter is a photoreceptor-specific promoter. In embodiments, the photoreceptor-specific promoter is selected from the group consisting of a rhodopsin kinase (RK) promoter, a rhodopsin (Rho) promoter, a beta phosphodiesterase (PDE) promoter, and a retinitis pigmentosa (RP1) promoter.

In embodiments, the promoter is a CBh promoter comprising a sequence having at least 90%, at least 95%, at least 97%, at least 98%, at least 99%, or 100% identity to SEQ ID NO: 154.

In embodiments, the promoter is a RK promoter comprising a sequence having at least 90%, at least 95%, at least 97%, at least 98%, at least 99%, or 100% identity to SEQ ID NO: 196. In embodiments, the promoter is a Rho promoter comprising a sequence having at least 90%, at least 95%, at least 97%, at least 98%, at least 99%, or 100% identity to SEQ ID NO: 197. In embodiments, the promoter is a PDE promoter comprising a sequence having at least 90%, at least 95%, at least 97%, at least 98%, at least 99%, or 100% identity to SEQ ID NO: 198.

In embodiments, the nucleic acid comprises an IRBP enhancer sequence upstream of the promoter. In embodiments, the IRBP enhancer sequence has at least 90%, at least 95%, at least 97%, at least 98%, at least 99%, or 100% identity to SEQ ID NO: 199. In embodiments, the IRBP enhancer sequence is located immediately upstream of the promoter, without any additional nucleotide in between.

In embodiments, the AAV vector genome comprises a CVA-MVM intron sequence having at least 90%, at least 95%, at least 97%, at least 98%, at least 99%, or 100% identity to SEQ ID NO: 226. In embodiments, the CVA-MVM intron sequence is located between the promoter and the heterologous nucleic acid encoding RS1.

In embodiments, the AAV vector genome comprises an intron sequence having at least 90%, at least 95%, at least 97%, at least 98%, at least 99%, or 100% identity to SEQ ID NO: 200 or 222. In embodiments, the intron is located between the promoter and the heterologous nucleic acid encoding RS1.

In embodiments, the heterologous nucleic acid encoding RS1 comprises a sequence having at least 90%, at least 95%, at least 97%, at least 98%, at least 99%, or 100% identity to SEQ ID NO: 117. In embodiments, the heterologous nucleic acid encodes an RS1 protein comprising an amino acid sequence having at least 90%, at least 95%, at least 97%, at least 98%, at least 99%, or 100% identity to SEQ ID NO: 143.

In embodiments, the polyadenylation signal comprises a sequence having at least 90%, at least 95%, at least 97%, at least 98%, at least 99%, or 100% identity to SEQ ID NO: 201 or 225.

In embodiments, the AAV vector genome does not comprise any telomeric repeats sequence.

In embodiments, the AAV vector genome comprises a first telomeric repeats sequence located between the polyadenylation signal and the second AAV inverted terminal repeat. In embodiments, the first telomeric repeats sequence has at least 90%, at least 95%, at least 97%, at least 98%, at least 99%, or 100% identity to SEQ ID NO: 203.

In embodiments, the AAV vector genome comprises a human beta-globin scaffold/matrix attachment region (βGlo_s/MAR) sequence. In embodiments, the βGlo_s/MAR sequence is located between the polyadenylation signal and the second AAV inverted terminal repeat. In embodiments, the βGlo_s/MAR sequence is located between the polyadenylation signal and the first telomeric repeats sequence. In embodiments, the βGlo_s/MAR sequence has at least 90%, at least 95%, at least 97%, at least 98%, at least 99%, or 100% identity to SEQ ID NO: 221.

In embodiments, the first AAV inverted terminal repeat comprises or consists of a sequence having at least 90%, at least 95%, at least 97%, at least 98%, at least 99%, or 100% identity to SEQ ID NO: 255. In embodiments, the second AAV inverted terminal repeat comprises or consists of a sequence having at least 90%, at least 95%, at least 97%, at least 98%, at least 99%, or 100% identity to SEQ ID NO: 256.

In embodiments, the AAV vector genome comprises, in 5′ to 3′ orientation: (a) the first AAV inverted terminal repeat, (b) the IRBP enhancer sequence having at least 90%, at least 95%, at least 97%, at least 98%, at least 99%, or 100% identity to SEQ ID NO: 199, (c) the RK promoter comprising a sequence having at least 90%, at least 95%, at least 97%, at least 98%, at least 99%, or 100% identity to SEQ ID NO: 196, (d) the heterologous nucleic acid encoding RS1, (e) the polyadenylation signal comprising a sequence having at least 90%, at least 95%, at least 97%, at least 98%, at least 99%, or 100% identity to SEQ ID NO: 225, and (f) the second AAV inverted terminal repeat.

In embodiments, the nucleic acid comprises an intron sequence having at least 90%, at least 95%, at least 97%, at least 98%, at least 99%, or 100% identity to SEQ ID NO: 222. In embodiments, the intron sequence is located between the promoter and the heterologous nucleic acid encoding RS1.

In embodiments, the nucleic acid comprises a CBA sequence of SEQ ID NO: 229, or a sequence having at most 5, at most 4, at most 3, at most 2, or at most 1 mutation(s) thereto. In embodiments, the CBA sequence is located immediately upstream of the intron sequence without any additional nucleotides in between. In embodiments, the nucleic acid comprises a CBA-MVM intron sequence having at least 90%, at least 95%, at least 97%, at least 98%, at least 99%, or 100% identity to SEQ ID NO: 226. In embodiments, the CVA-MVM intron sequence is located between the promoter and the heterologous nucleic acid encoding RS1.

In embodiments, the nucleic acid comprises a sequence having at least 90%, at least 95%, at least 97%, at least 98%, at least 99%, or 100% identity to SEQ ID NO: 224.

In embodiments, the nucleic acid comprises a polynucleotide sequence having at least 90%, at least 95%, at least 97%, at least 98%, at least 99%, or 100% identity to any one of SEQ ID NO: 224 and 240-252.

In one aspect, the disclosure provides nucleic acids comprising, in 5′ to 3′ orientation: (a) a promoter, (b) a heterologous nucleic acid encoding a transgene, and (c) a polyadenylation signal, wherein the promoter is a CBh promoter comprising a sequence having at least 90%, at least 95%, at least 97%, at least 98%, at least 99%, or 100% identity to SEQ ID NO: 154, and wherein the polyadenylation signal comprises a sequence having at least 90%, at least 95%, at least 97%, at least 98%, at least 99%, or 100% identity to SEQ ID NO: 201 or 225.

In embodiments, the nucleic acid comprises an intron sequence having at least 90%, at least 95%, at least 97%, at least 98%, at least 99%, or 100% identity to SEQ ID NO: 200, 222, 226, or 227. In embodiments, the intron is located between the promoter and the heterologous nucleic acid encoding the transgene.

In embodiments, the nucleic acid comprises a first ITR that is located 5′ to the promoter, and a second ITR that is located 3′ to the polyadenylation signal.

In embodiments, the nucleic acid does not comprise any telomeric repeats sequence.

In embodiments, the nucleic acid comprises a first telomeric repeats sequence located between the polyadenylation signal and the second AAV ITR. In embodiments, the first telomeric repeats sequence has at least 90%, at least 95%, at least 97%, at least 98%, at least 99%, or 100% identity to SEQ ID NO: 202. In embodiments, the nucleic acid comprises a second telomeric repeats sequence located between the first AAV inverted terminal repeat and the promoter. In embodiments, the second telomeric repeats sequence has at least 90%, at least 95%, at least 97%, at least 98%, at least 99%, or 100% identity to SEQ ID NO: 203.

In one aspect, the disclosure provides vectors comprising the nucleic acid of the disclosure.

In one aspect, the disclosure provides AAV vector genomes comprising the nucleic acid of the disclosure.

In one aspect, the disclosure provides AAV viral vectors comprising the AAV vector genome of the disclosure. In embodiments, the AAV viral vector comprises the AAV capsid protein comprising an amino acid sequence that is at least 95%, at least 98%, at least 99%, at least 99.5%, or 100% identical to any one of SEQ ID NO: 1-3, 30-34, 49, 84 and 164.

In one aspect, the disclosure provides methods of expressing a transgene in a retinal cell, comprising delivering the nucleic acid of the disclosure to the retinal cell, or transducing the retinal cell with the AAV viral vector of the disclosure. In embodiments, the retinal cell is a retinal ganglion cell.

In one aspect, the disclosure provides methods of treating a disease or disorder comprising administering the AAV viral vector of the disclosure to a subject. In embodiments, the AAV viral vector is administered to the subject intra-ocularly, peri-ocularly, intravitreally, para-retinally, or sub-retinally. In embodiments, the disease or disorder is macular degeneration, retinitis pigmentosa, Autosomal Dominant Optic Atrophy, Retinoschisis, Stargardt disease, Bietti's Crystalline Dystrophy or BEST vitelliform macular dystrophy. In embodiments, the disease or disorder is X-linked Retinoschisis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustration of different modes of intraocular administrations (adapted from Yiu et al., Mol Ther Methods Clin Dev. 2020 Jan. 21; 16:179-191, the content of which is incorporated by reference herein in its entirety.)

FIGS. 2A-2E show AAV viral vector-mediated GFP expression in the eyes of a non-human primate animal model via intravitreal or para-retinal administrations. Scanning laser ophthalmoscopy (SLO) imaging was performed at Day 26 after injection of the indicated AAV viral vector. FIG. 2A shows transduction spread mediated by intravitreal injection of AAV viral vector comprising AAV204 capsid protein. FIG. 2B shows transduction spread mediated by para-retinal injection of AAV viral vector comprising AAV204 capsid protein. FIG. 2C shows transduction spread mediated by para-retinal injection of AAV viral vector comprising AAV8 capsid protein. FIG. 2D shows transduction spread mediated by para-retinal injection of AAV viral vector comprising AAV214 capsid protein. FIG. 2E shows transduction spread mediated by para-retinal injection of AAV viral vector comprising AAV214-D5 capsid protein.

FIGS. 3A-3F show imaging analysis of retinas after AAV administration. FIG. 3A shows the composite images of retina after AAV204 intravitreal administration. FIG. 3B shows the composite (upper left), rhodopsin (upper right), and zoom-in composite (lower) images of retina after AAV204 para-retinal administration. FIG. 3C shows the composite (upper left), rhodopsin (upper right), and zoom-in composite (lower) images of retina after AAV204 para-retinal administration. FIG. 3D shows the immunohistochemistry analysis of rhodopsin and GFP expression 1-month post para-retinal injection of AAV204 or AAV8 viral vector. FIG. 3E shows rhodopsin and GFP expression in the fovea post para-retinal injection of AAV204 viral vector. FIG. 3F shows rhodopsin and GFP expression along the papillomacular bundle post para-retinal injection of AAV204 viral vector.

FIGS. 4A-4C show AAV viral vector-mediated GFP expression in the eyes of a non-human primate animal model via sub-retinal administration. SLO imaging was performed at Day 27 after injection of the indicated AAV viral vector. FIG. 4A shows transduction spread mediated by sub-retinal injection of AAV viral vector comprising AAV8 capsid protein. FIG. 4B shows transduction spread mediated by sub-retinal injection of AAV viral vector comprising AAV214 capsid protein. FIG. 4C shows transduction spread mediated by sub-retinal injection of AAV viral vector comprising AAV214-D5 capsid protein. FIG. 4D shows the composite (upper left), rhodopsin (upper right), and zoom-in composite (lower) images of retina after AAV8 subretinal administration. FIG. 4E shows the composite (upper left), rhodopsin (upper right), and zoom-in composite (lower) images of retina after AAV214 subretinal administration. FIG. 4F shows the composite (upper left), rhodopsin (upper right), and zoom-in composite (lower) images of retina after AAV214-D5 subretinal administration.

FIG. 5 shows a diagram of the VP1, VP2, and VP3 portions of the capsid protein. The VP1- and VP2-specific portions are indicated along with the VP3 portion, which is identical to the VP3 protein produced. The amino acid sequence of AAV214 VP3 (SEQ ID NO: 41) is shown and variable regions I-IX are indicated. The full VP1 protein amino acid sequence for AAV214 is provided as SEQ ID NO: 3.

FIG. 6 shows an alignment of the VP1 protein amino acid sequences of AAV214 (SEQ ID NO: 3) and AAV214-D5 (SEQ ID NO: 164).

FIG. 7 shows the design of various Opa1-encoding AAV vector genomes.

FIG. 8A shows Opa1 expression in 293 cells transfected with each indicated vector.

FIG. 8B is a chart showing the virus production yield of each indicated vector.

FIG. 9A and FIG. 9B show Opa1 expression in viral potency tests. FIG. 9C shows protein staining results of cells transfected with each indicated vector.

FIG. 10A shows a schematic diagram of the proof-of-concept study that evaluates AAV204 viral vectors encoding Opa1. FIG. 10B shows Western analysis of Opa1 and FLAG-tag expression in heterozygous treated mice. FIG. 10C shows Western analysis of Opa1, FLAG-tag, Brn3a and Rho expression. FIG. 10D shows RT-PCR analysis of RNA transcript levels of human Opa1, mouse Opa1, and FLAG-tag in wild-type or Opa1 heterozygous, untreated or treated animals at 2 months post-injection.

FIG. 11A and FIG. 11B show schematics of the proof-of-concept (POC) studies to evaluate AAV204 viral vectors encoding Opa1.

FIG. 12A shows a table summary of various RS1-encoding AAV vector genomes.

FIG. 12B shows the design of various RS1-encoding AAV vector genomes.

FIG. 13 shows Western analysis of RS1 protein expression.

FIG. 14A is a chart showing expression of secreted RS1 protein in Lec2 cells transduced with each indicated AAV viral vector. FIG. 14B is a chart showing mRNA expression of target transgene in Lec2 cells transduced with each indicated AAV viral vector. FIG. 14C shows Western analysis of secreted RS1 protein in Lec2 cells transduced with each indicated AAV viral vector. FIG. 14D shows Western analysis comparing the molecular weight of the myc-tagged RS1 and wildtype RS1.

FIG. 15A shows a diagram of the proof-of-concept (POC) studies to evaluate AAV204 viral vectors encoding RS1. FIG. 15B is a chart showing RS1 protein expression in wild-type mice transduced with the indicated AAV.

FIG. 16A and FIG. 16B show Western analysis of RS1 expression in mice transduced with the indicated AAV.

FIG. 17A and FIG. 17B show schematics of the proof-of-concept (POC) studies to evaluate AAV204 viral vectors encoding RS1.

FIG. 18A is a table showing the different treatment groups for the mouse study of RS1 expression. FIG. 18B shows endogenous mouse RS1 (mRs1) expression as measured by qPCR for Groups 5-8 at the 2 months post-treatment (mpt) timepoint. FIG. 18C shows RS1 expression from virally delivered transgene at the 2 mpt timepoint. The data are log-transformed to facilitate visualization of comparisons with large differences. nd: not detected. FIG. 18D and FIG. 18E show the results of simultaneous detection of endogenous Rs1 and viral-derived myc-RS1 using a highly specific RS1 antibody. The Western analysis is shown in FIG. 18D and quantified in FIG. 18E. The RS1-specific bands in Groups 5 and 6 are larger than the endogenous protein in Group 8 due to the presence of the myc tag. A myc-RS1 positive control, derived from transduced tissue culture cells, is included in the final lane. nd: not detected.

FIG. 19A shows IHC staining of wildtype (WT) retina at 2 mpt timepoint. FIG. 19B shows IHC staining of untreated mutant retina at 2 mpt timepoint. FIG. 19C shows IHC staining of mutant retina transduced with RS1_46 at 2 mpt timepoint. FIG. 19D shows IHC staining of mutant retina transduced with RS1_46 in the right eye of animal #123 at 2 mpt timepoint. FIG. 19E shows IHC staining of mutant retina transduced with RS1_48 at 2 mpt timepoint. FIG. 19F is a chart showing cone density analysis at p90, 2 mpt timepoint. FIG. 19G shows staining of retina samples. The upper panels A-C show retina samples transduced with AAV204.RK:RS1_28: RS1 is stained in red, and PNA is stained in green to demonstrate the extent of cone degeneration. The bottom panels are higher magnification images showing cone density in a similarly prepared WT retina (section D) and the transduced and untransduced regions of the treated mutant retina (sections E and F, respectively).

FIG. 20A shows Western analysis of RS1 protein expression. The positive control in the last lane is recombinant RS1 from transfected tissue culture cells. FIG. 20B is a chart showing quantification of band intensity. nd=not detected. FIG. 20C shows IHC of WT retina at 6 mpt timepoint. RS1 expression (red) in WT eyes (group 3) is uniform across the retina and concentrated in photoreceptors. Panels to the right show a higher magnification view of the boxed region. FIG. 20D shows IHC of mutant retina at 6 mpt timepoint. RS1 staining (red) is absent in mutant retinas (group 1). Panels to the right show higher magnification views of the boxed region. In the absence of RS1, retinal vasculature is labeled with the secondary antibody (arrows). FIG. 20E shows IHC of mutant retina treated with RS1_28 at 6 mpt timepoint. RS1 expression (red) is visible in the dorsal retina of this eye from group 2. Higher magnification views of transduced (a) and non-transduced (b) areas are shown to the right. FIG. 20F shows IHC of mutant retina treated with RS1_26 at 6 mpt timepoint. None of the eyes in this group had detectable RS1 expression.

FIG. 21 is a chart showing quantification of cone density at 6 mpt timepoint. Cone density was measured from sections stained with peanut agglutinin (PNA). Measurements from group 2 are segregated by RS1 expression. Within group 2, individual data points of identical color reflect the RS1-positive and -negative areas of the same section. In four of the five eyes with detectable RS1 expression, the cone density was slightly higher in the area with adjacent RS1 expression. Eyes that had no detectable RS1 expression are represented with black dots.

FIG. 22A shows OCT imaging of representative eyes at the 6 mpt timepoint. Yellow bars in each image indicate the thickness of the ONL. Arrows show examples of retinal schises that were present in one eye. FIG. 22B is a chart showing quantification analysis of ONL measurements across all groups.

FIG. 23A shows IHC of WT retina at 6 mpt timepoint. FIG. 23B shows IHC of untreated mutant retina at 6 mpt timepoint. FIG. 23C shows IHC of mutant retina treated with AAV204.CBh:RS1_16 at 6 mpt timepoint. In most eyes treated with AAV204.CBh:RS1_16, a large area of extensive degeneration was seen in the dorsal retina where the bleb would have been. Visible RS1 expression was rare and is not present in this eye. The boxed area, enlarged in the panels on the right, shows improved cone density (PNA staining) despite there being no detectable RS1 staining. FIG. 23D shows IHC of mutant retina treated with AAV204.CBh:RS1_16 at 6 mpt timepoint in one particular eye (with injection injury). On the left, the asterisk shows treated retina with severe injection related injury and RS1 expression (red) throughout the inner retina. On the right shows a deeper section of the same eye showing inner retinal RS1 expression (B′) as well as expression in photoreceptors in the area adjacent to the lesion (B″). In all panels, PNA (gray) labels cone outer segments and Iba1 (green) labels inflammatory cells. FIG. 23E shows IHC of mutant retina treated with AAV204.CBh:RS1_18 at 6 mpt timepoint. In this example, strong RS1 expression was observed in the dorsal photoreceptors and extended into the inner retina (box a). In contrast, RS1 staining in the ventral retina (box b) was absent. Most of the dorsal retina was devoid of cones despite being otherwise structurally intact. However, the area just dorsal to box b was RS1-positive and also cone-enriched compared to the adjacent RS1-negative area. FIG. 23F is a chart summarizing the cone density analysis at 6 mpt timepoint. Cone density was significantly improved in all treated eyes, even in areas where RS1 immunostaining is not detectable. For this analysis, areas of severe degeneration and areas of cone depletion were omitted.

FIG. 24A is a chart summarizing mean ONL thickness among all groups. For treated eyes, separate measurements were taken for RS1+ and RS1− areas of the retina, and data points of similar color within each group reflect measurements taken from the same section. Areas of severe degeneration were not included. Data from representative eyes in Groups 10 and 11 are shown in FIG. 24B and FIG. 24C, where star shaped labels indicate areas of treated retinas with RS1 expression.

FIG. 25A shows Western analysis of RS1 protein expression. The positive control in the last lane is recombinant RS1 from transfected tissue culture cells. FIG. 25B is a chart showing quantification of band intensity. nd=not detected.

FIG. 26A shows OCT imaging of an untreated mutant eye. FIG. 26B shows OCT imaging of a wildtype eye. FIG. 26C shows OCT imaging of a post-operative bleb to confirm a successful injection. FIG. 26D shows OCT imaging of a treated eye 6 months post-injection. FIG. 26E shows OCT imaging of a treated eye 6 months post-injection; here the margin of the bleb was captured. FIG. 26F shows OCT imaging of a treated eye 6 months post-injection. Yellow lines in these figures indicate the thickness of the ONL. FIG. 26G is a chart showing quantification of ONL thickness. FIG. 26H is a paired t-test estimation plot showing the increased ONL thickness in the treated dorsal retina (11D) compared to the untreated ventral retina in the same eye (11V).

FIG. 27 shows representative flicker ERG at 6 mpt timepoint.

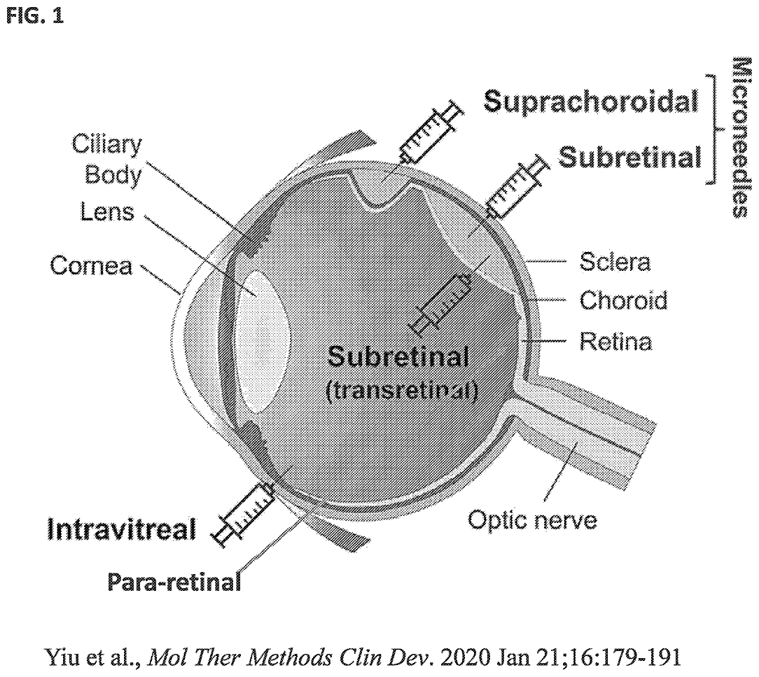

### DETAILED DESCRIPTION

Some embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

Unless the context indicates otherwise, it is specifically intended that the various features of the invention described herein can be used in any combination. Moreover, the disclosure also contemplates that in embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols in Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (RI. Freshney, ed. (1987)).

It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 10% of the specified amount.

The terms “acceptable”, “effective”, or “sufficient” when used to describe the selection of any components, ranges, dose forms, etc. disclosed herein intend that said component, range, dose form, etc. is suitable for the disclosed purpose.

Unless specifically recited, the term “host cell” includes a eukaryotic host cell, including, for example, fungal cells, yeast cells, higher plant cells, insect cells and mammalian cells. Non-limiting examples of eukaryotic host cells include simian, bovine, porcine, murine, rat, avian, reptilian and human, e.g., HEK293 cells and 293T cells.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

“Under transcriptional control” is a term well understood in the art and indicates that transcription of a polynucleotide sequence, usually a DNA sequence, depends on its being operatively linked to an element that contributes to the initiation of, or promotes, transcription. “Operatively linked” intends that the polynucleotides are arranged in a manner that allows them to function in a cell. In one aspect, this invention provides promoters operatively linked to the downstream sequences.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide and/or a fragment thereof. The antisense strand is the complement of such a nucleic acid, and the encoding sequence can be deduced therefrom.

The term “promoter” as used herein means a control sequence that is a region of a polynucleotide sequence at which the initiation and rate of transcription of a coding sequence, such as a gene or a transgene, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, an H1 promoter, a ubiquitous chicken β-actin hybrid (CBh) promoter, a small nuclear RNA (U1a or U1b) promoter, an MeCP2 promoter, an MeP418 promoter, an MeP426 promoter, a minimal MeCP2 promoter, a VMD2 promoter, an mRho promoter or an EFI promoter.

Additional non-limiting exemplary promoters provided herein include, but are not limited to EFla, Ubc, human β-actin, CAG, TRE, Ac5, Polyhedrin, CaMKIIa, Gal1, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In embodiments, the promoter is used together with an enhancer to increase the transcription efficiency. Non-limiting examples of enhancers include an interstitial retinoid-binding protein (IRBP) enhancer, an RSV enhancer or a CMV enhancer.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise, consist essentially of, or consist of a protein's or peptide's sequence. As used herein the term “amino acid” refers to either natural and/or unnatural or synthetic amino acids, including glycine and both the D and L optical isomers, amino acid analogs and peptidomimetics.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In embodiments, the signal peptide is removed following localization. Examples of signal peptides are well known in the art. Non-limiting examples are those described in U.S. Pat. Nos. 8,853,381, 5,958,736, and 8,795,965. In embodiments, the signal peptide can be an IDUA signal peptide.

The terms “equivalent” or “biological equivalent” are used interchangeably when referring to a particular molecule, biological material, or cellular material and intend those having minimal homology while still maintaining desired structure or functionality. Non-limiting examples of equivalent polypeptides include a polypeptide having at least about 60%, at least about 65%, at least about 70%, at least about 75%, at least about 80%, at least about 85%, at least about 90%, at least about 95% identity or at least about 99% identity to a reference polypeptide (for instance, a wild-type polypeptide); or a polypeptide which is encoded by a polynucleotide having at least about 70%, at least about 75%, at least about 80%, at least about 85%, at least about 90%, at least about 95% identity, at least about 97% sequence identity or at least about 99% sequence identity to the reference polynucleotide (for instance, a wild-type polynucleotide).

“Homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using ClustalW (available at genome.jp/tools-bin/clustalw/) and Gonnet (for protein) weight matrix. In embodiments, the ClustalW parameters used for performing nucleic acid sequence alignments using the nucleic acid sequences found herein are generated using the ClustalW (for DNA) weight matrix.

As used herein, amino acid modifications may be substitutions, deletions or insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g., charge, hydrophobicity or size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one charged or polar residue for another, such as the substitution of arginine for lysine, glutamic acid for aspartic acid, glutamine for asparagine, and the like. Other illustrative examples of conservative substitutions include the changes of: alanine to serine; asparagine to glutamine or histidine; aspartate to glutamate; cysteine to serine; glycine to proline; histidine to asparagine or glutamine; lysine to arginine, glutamine, or glutamate; phenylalanine to tyrosine, serine to threonine; threonine to serine; tryptophan to tyrosine; tyrosine to tryptophan or phenylalanine; and the like.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally-occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

A “viral vector” is defined as a recombinantly produced virus or viral particle that contains a polynucleotide to be delivered into a host cell, either in vivo, ex vivo or in vitro. Examples of viral vectors include retroviral vectors, AAV viral vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5 (7): 823-827.

As used herein, the term “recombinant expression system” or “recombinant vector” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; bacteria; viruses, such as baculoviruses, adenoviruses and retroviruses; bacteriophage, cosmid, plasmid, and fungal vectors; and other recombination vehicles typically used in the art which have been described for expression in a variety of eukaryotic and prokaryotic hosts, and may be used for gene therapy as well as for simple protein expression. Liposomes that also comprise, consist essentially of, or consist of a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized prior to introduction into the host cell.

“Plasmids” used in genetic engineering are called “plasmid vectors”. Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, consisting essentially of, or consisting of the viral genome or part thereof, and a transgene.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VPl, VP2 and VP3. In embodiments, the AAV refers to the serotype AAV1, AAV2, AAV4, AAV5, AAV6, AAV7, AAV8, AAV9, AAV10, AAV11, AAV12, AAV13, AAVPHP.B, or AAVrh74.

An “AAV vector” as used herein refers to a vector comprising one or more heterologous nucleic acid (HNA) sequences and one or more AAV inverted terminal repeat sequences (ITRs). Such AAV vectors can be replicated when present in a host cell that provides the functionality of rep and cap gene products, and allow the ITRs and the nucleic acid between the ITRs to be packaged into infectious viral particles. In embodiments, AAV vectors comprise a promoter, at least one nucleic acid that may encode at least one protein or RNA, and/or an enhancer and/or a terminator within the flanking ITRs that is packaged into the infectious AAV particle. The ITRs and the nucleic acid between the ITRs can be encapsidated into the AAV capsid, and this encapsidated portion of the nucleic acid may be referred to as the “AAV vector genome”. AAV vectors may contain elements in addition to the encapsidated portion, for example, antibiotic resistance genes or other elements known in the art included in the plasmid for manufacturing purposes but not packaged into the AAV particle.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz J E, Samulski R J (December 2000). “Building a better vector: the manipulation of AAV virions”. Virology. 278 (2): 301-8, each of which is incorporated herein by reference in its entirety.

An “AAV virion” or “AAV viral particle” or “AAV viral vector” or “AAV vector particle” or “AAV particle” refers to a viral particle composed of at least one AAV capsid protein and an encapsidated AAV vector genome.

As used herein, the term “helper” in reference to a virus or plasmid refers to a virus or plasmid used to provide the additional components necessary for replication and packaging of any one of the AAV vector genomes disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In embodiments, the pHELP plasmid may be the pHELPK plasmid, wherein the ampicillin expression cassette is exchanged with a kanamycin expression cassette; pHELPK has the sequence shown in SEQ ID NO: 92.

As used herein, a packaging cell (or a helper cell) is a cell used to produce viral vectors. Producing recombinant AAV viral vectors requires Rep and Cap proteins provided in trans as well as gene sequences from Adenovirus that help AAV replicate. In some aspects, packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

As used herein, a reporter protein is a detectable protein that is operably linked to a promoter to assay the expression (for example, tissue specificity and/or strength) of the promoter. In aspects, a reporter protein may be operably linked to a polypeptide. In aspects, reporter proteins may be used in monitoring DNA delivery methods, functional identification and characterization of promoter and enhancer elements, translation and transcription regulation, mRNA processing and protein: protein interactions. Non-limiting examples of a reporter protein are β-galactosidase; a fluorescent protein, such as, Green Fluorescent Protein (GFP) or Red Fluorescent Protein (RFP); luciferase; glutathione S-transferase; and maltose binding protein.

A “pharmaceutical composition” is intended to include the combination of an active ingredient such as a polypeptide, a polynucleotide, an antibody, or a viral vector, with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject” of diagnosis or treatment is a cell or an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In embodiments, the subject is a human.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected), or a group of tissues making up an organ or part or region of the body of an organism. The tissue may comprise, consist essentially of, or consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, in embodiments the effective amount is the amount sufficient to result in regaining part or full function of a gene that is deficient in a subject. In embodiments, the effective amount of an AAV viral particle is the amount sufficient to result in expression of a gene in a subject. The skilled artisan will be able to determine appropriate amounts depending on these and other factors.

In embodiments, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

### AAV Structure and Function

AAV is a replication-deficient parvovirus, the single-stranded DNA genome of which is about 4.7 kb in length, including two about 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). The sequence of the AAV rh.74 genome is provided in U.S. Pat. No. 9,434,928, incorporated herein by reference in its entirety. U.S. Pat. No. 9,434,928 also provide the sequences of the capsid proteins and a self-complementary genome. In one aspect, the genome is a self-complementary genome. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus polyadenylation signal is located at map position 95 of the AAV genome. The life cycle and genetics of AAV are reviewed in Muzyczka, Current Topics in Microbiology and Immunology, 158:97-129 (1992).

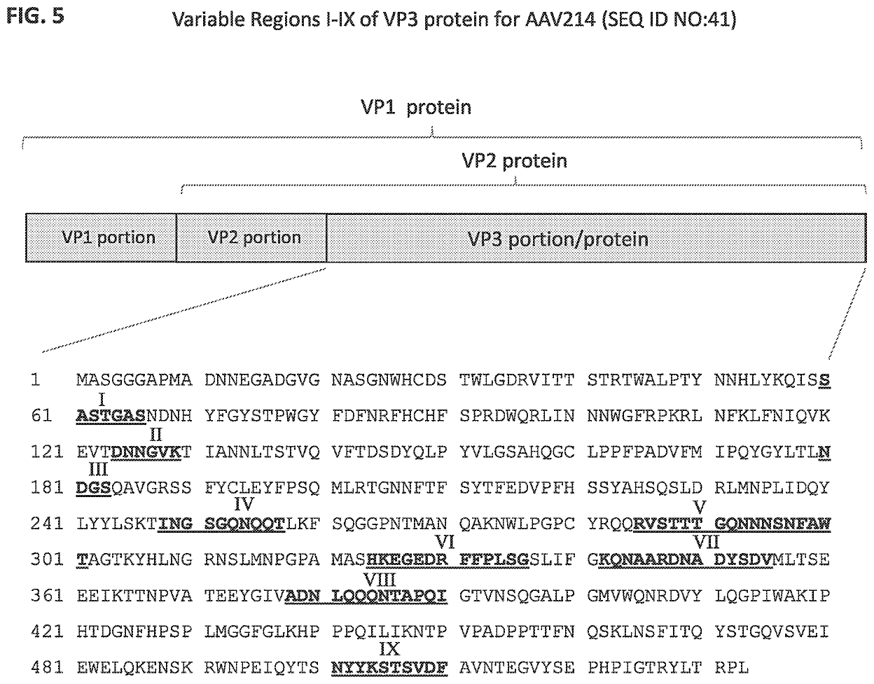

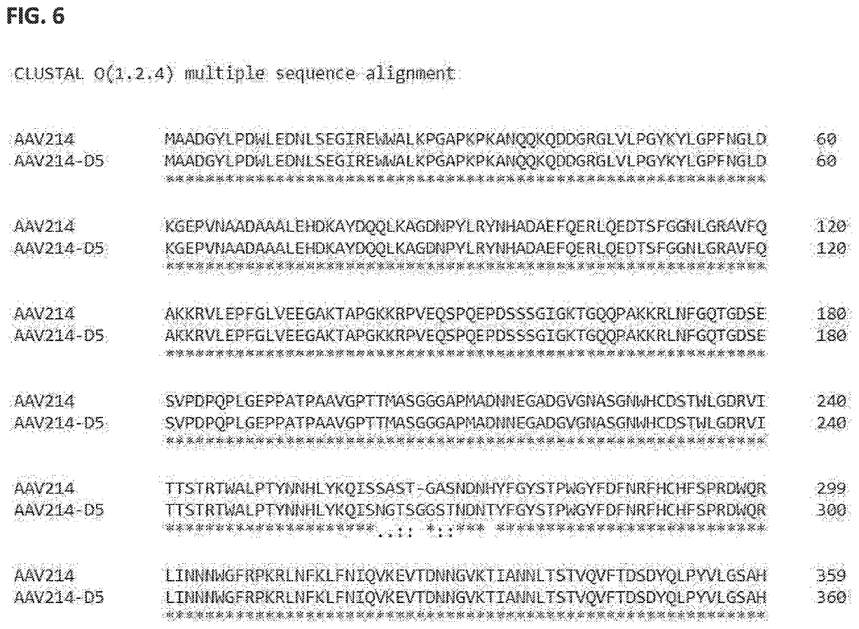

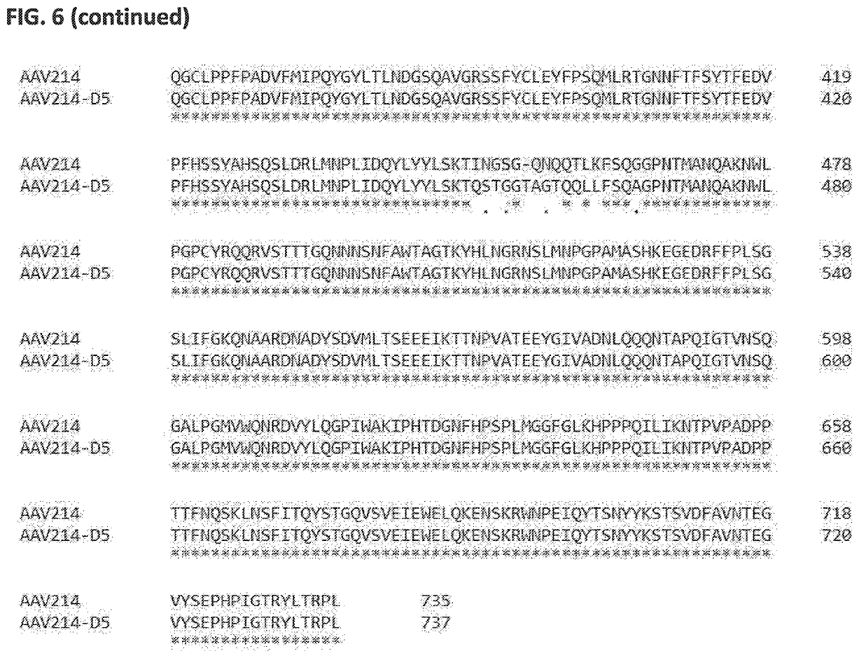

Each VP1 protein contains a VP1 portion, a VP2 portion and a VP3 portion. The VP1 portion is the N-terminal portion of the VP1 protein that is unique to the VP1 protein, corresponding to the amino acids 1-137 portion of SEQ ID NO: 164. The VP2 portion is the amino acid sequence present within the VP1 protein that is also found in the N-terminal portion of the VP2 protein, corresponding to the amino acids 138-202 portion of SEQ ID NO: 164. The VP3 portion and the VP3 protein have the same sequence. The VP3 portion is the C-terminal portion of the VP1 protein that is shared with the VP1 and VP2 proteins, corresponding to the amino acids 203-737 portion of SEQ ID NO: 164. See FIG. 5.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958 June 2012, the contents of which are incorporated herein by reference.

AAV possesses unique features that make it attractive as a viral vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vector genomes. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8:659-669 (1997); Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise, consist essentially of, or consist of a nucleic acid molecule encoding a therapeutic protein (e.g., CYP4V2, RS1, PDE6B, ABCA4, BEST1, OPA1 or OPA3) and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV PHP.B and AAV rh74. Production of pseudotyped rAA Vis disclosed in, for example, WO2001083692. Other types of rAAV variants, for example rAAV with capsid mutations, are also contemplated. See, e.g., Marsic et al., Molecular Therapy, 22 (11): 1900-1909 (2014). The nucleotide sequences of the genomes of various AAV serotypes are known in the art.

### AAV Vector Particles, Capsid Proteins, and AAV Vectors

Provided herein are AAV vector particles, AAV vectors, and capsid proteins that have desirable tissue specificity and find use in delivering a variety of therapeutic payloads, including nucleic acids, and proteins useful in the treatment of disease.

### AAV Capsid Proteins

The disclosure provides AAV particles possessing properties of high gene transfer efficiency and increased tissue tropism. AAV viral vector delivery currently relies on the use of serotype selection for tissue targeting based on the natural tropism of the virus or by the direct injection into target tissues. Many currently available AAV viral vectors are, however, suboptimal for delivering genes to a specific target site.

The present disclosure provides AAV capsid protein sequences that confer high gene transfer efficiency and increased tissue specificity on the AAV particles comprising them. In embodiments, the AAV particles comprising such AAV capsid proteins are administered via a specific delivery route to achieve optimal delivery to specific target site.

In embodiments, the VP1 capsid protein comprises any one of the amino acid sequences listed in Table 1, or a sequence having up to 1, 2, 3, 4, 5, 6, 7, 8, 9, or 10 amino acids mutated, deleted or added as compared to, any one of the amino acid sequences listed in Table 1. In embodiments, up to 15 amino acids, up to 20 amino acids, up to 30 amino acids, or up to 40 amino acids may be mutated, deleted or added compared to these sequences. In embodiments, the VP1 capsid protein is encoded by any one of the nucleic acid sequences listed in Table 1, or a sequence having up to 5, up to 10, up to 30, or up to 60 nucleotide changes to any one of the nucleic acid sequences listed in Table 1.

TABLE 1
VP1 Capsid Proteins

| Amino Acid SEQ ID NO: | NA SEQ ID NO: | AAV Capsid Name |
|---|---|---|
| 1 | 98 | AAV 110 |
| 2 | 15 | AAV 204 |
| 3 | 18 | AAV 214 |
| 30 | 19 | AAV 214A |
| 31 | 20 | AAV 214e |
| 32 | 21 | AAV 214e8 |
| 33 | 22 | AAV 214e9 |
| 34 | 23 | AAV 214e10 |
| 49 | 47 | AAV ITB102_45 |
| 84 | 82 | AAV 214AB |
| 164 | 167 | AAV 214-D5 |

In embodiments, the AAV VP1 protein comprises, consists essentially of, or consists of an amino acid sequence of SEQ ID NOs: 1-3, 30-34, 49, 84 or 164, or a sequence having up to 1, 2, 3, 4, 5, 6, 7, 8, 9, or 10 amino acids different from SEQ ID NOs: 1-3, 30-34, 49, 84 or 164. Also provided are polynucleotides encoding these VP1 proteins. In embodiments, the polynucleotides encoding the VP1 proteins comprise, consist essentially of, or consist of the sequence of SEQ ID NOs: 15, 18-23, 47, 82, 98 or 167 or a sequence having up to 5, up to 10, or up to 30 nucleotide changes to SEQ ID NOs: 15, 18-23, 47, 82, 98 or 167.

In embodiments, the AAV capsid sequence is an AAV-110 capsid protein (SEQ ID NO: 1), AAV204 capsid protein (SEQ ID NO: 2), AAV214 capsid protein (SEQ ID NO: 3) or AAV ITB102_45 capsid protein (SEQ ID NO: 49). In embodiments, the AAV capsid protein is a variant of the AAV214 capsid protein. In embodiments, the AAV capsid protein is AAV214A (SEQ ID NO: 30), AAV-214-AB (SEQ ID NO: 84), AAV214e (SEQ ID NO: 31), AAV214e8 (SEQ ID NO: 32), AAV214e9 (SEQ ID NO: 33), AAV214e10 (SEQ ID NO: 34), or AAV214-D5 (SEQ ID NO: 164). In embodiments, the AAV capsid protein is AAV214-D5 (SEQ ID NO: 164).

In embodiments, the AAV capsid sequence is an AAV204 capsid protein (SEQ ID NO: 2), AAV214 capsid protein (SEQ ID NO: 3), AAV214-D5 capsid protein (SEQ ID NO: 164) or AAV8 capsid protein (SEQ ID NO: 67).

Sequences for exemplary VP2 and VP3 proteins are provided in Table 2 and Table 3. Given the VP2 and VP3 sequences, the VP1 portions may be determined by alignment with the full, VP1 protein sequence.

TABLE 2
VP2 Capsid Proteins

| Amino Acid SEQ ID NO: | Name |
|---|---|
| 35 | 214 |
| 36 | 214A |
| 37 | 214e |
| 38 | 214e8 |
| 39 | 214e9 |
| 40 | 214e10 |
| 85 | 214AB |
| 50 | ITB102_45 |
| 165 | 214-D5 |

In embodiments, the AAV VP2 proteins comprise, consist essentially of, or consist of an amino acid sequence of any one of SEQ ID NOs: 35-40, 50, 85 and 165, or a sequence having up to 1, 2, 3, 4, 5, 6, 7, 8, 9, or 10 amino acids different from SEQ ID NOs: 35-40, 50, 85 or 165. In embodiments, the AAV VP2 proteins comprise, consist essentially of, or consist of an amino acid sequence of SEQ ID NO: 165, or a sequence having up to 1, 2, 3, 4, 5, 6, 7, 8, 9, or 10 amino acids different from SEQ ID NO: 165.

Also provided are polynucleotides encoding these VP2 proteins. In embodiments, the polynucleotide encoding the VP2 protein comprises, consists essentially of, or consists of the sequence of SEQ ID NO: 47, or a sequence having up to 5, up to 10, or up to 30 nucleotide changes to SEQ ID NO: 47. In embodiments, the polynucleotide encoding the VP2 protein comprises, consists essentially of, or consists of the sequence of SEQ ID NO: 168, or a sequence having up to 5, up to 10, or up to 30 nucleotide changes to SEQ ID NO: 168.

Exemplary nucleic acids for the other capsid VP2 portions may be derived from the corresponding portions of the VP1 capsid protein nucleic acids.

TABLE 3
VP3 Capsid proteins

| Amino Acid SEQ ID NO: | NA SEQ ID NO: | AAV Capsid Name |
|---|---|---|
| 17 | 16 | 204 |
| 41 | 24 | 214 |
| 42 | 25 | 214A |
| 43 | 26 | 214e |
| 44 | 27 | 214e8 |
| 45 | 28 | 214e9 |
| 46 | 29 | 214e10 |
| 86 | 83 | 214AB |
| 51 | 48 | ITB102_45 |
| 166 | 169 | 214-D5 |

The VP3 proteins of AAV214, AAV214e, AAV214e8, AAV214e9, AAV214e10 have the same amino acid (SEQ ID NO:41) and nucleic acid (SEQ ID NO: 24) sequences.

In embodiments, the AAV VP3 proteins comprise, consist essentially of, or consist of the amino acid sequence of SEQ ID NOs: 17, 41-46, 51, 86, or 166, or a sequence having up to 1, 2, 3, 4, 5, 6, 7, 8, 9, or 10 amino acids different from SEQ ID NOs: 17, 41-46, 51, 86, or 166. In embodiments, the AAV VP3 proteins comprise, consist essentially of, or consist of the amino acid sequence of SEQ ID NO: 166, or a sequence having up to 1, 2, 3, 4, 5, 6, 7, 8, 9, or 10 amino acids different from SEQ ID NO: 166.

Also provided are polynucleotides encoding these VP3 proteins. In embodiments, the polynucleotides encoding the proteins that comprise, consist essentially of, or consist of the sequence of SEQ ID NOs: 16, 24-29, 48, 83 or 169, or a sequence having up to 5, up to 10, or up to 30 nucleotide changes to SEQ ID NOs: 16, 24-29, 48, 83, or 169. In embodiments, the polynucleotides encoding the proteins that comprise, consist essentially of, or consist of the sequence of SEQ ID NO: 169, or a sequence having up to 5, up to 10, or up to 30 nucleotide changes to SEQ ID NO: 169.

In embodiments, the AAV capsid protein is a chimeric protein. In embodiments, a VP1, VP2, or VP3 portion of the AAV capsid protein disclosed herein may be replaced with a VP1, VP2, or VP3 portion from a different AAV capsid protein disclosed herein.

In embodiments, provided herein is an AAV capsid protein comprising a leucine residue at amino acid 129, an asparagine residue at amino acid 586 and a glutamic acid residue at amino acid 723, wherein amino acid positions in the AAV capsid protein are numbered with respect to amino acid positions in the amino acid sequence of SEQ ID NO: 2. In some cases, the protein comprises the amino acid sequence of SEQ ID NO: 2. In other cases, these amino acids may be introduced into other capsid proteins.

In embodiments, provided herein is an AAV VP1 capsid protein comprising a VP1 portion, a VP2 portion and a VP3 portion, wherein the VP1 portion comprises a leucine (L) residue at amino acid 129, wherein the VP2 portion comprises a threonine (T) or asparagine (N) residue at amino acid 157 and a lysine (K) or serine(S) residue at amino acid 162, and wherein the VP3 portion comprises asparagine (N) residue at amino acid 223, an alanine (A) residue at amino acid 224, a histidine (H) residue at amino acid 272, a threonine (T) residue at amino acid 410, a histidine (H) residue at amino acid 724 and a proline (P) residue at amino acid 734, wherein amino acid positions in the AAV capsid protein are numbered with respect to amino acid positions in the amino acid sequence of SEQ ID NO: 3 (i.e., VP1 capsid subunit numbering).

In embodiments, the VP1 portion further comprises an aspartic acid (D) or alanine (A) residue at amino acid 24, wherein amino acid positions in the AAV capsid protein are numbered with respect to amino acid positions in the amino acid sequence of SEQ ID NO: 3. In embodiments, the VP2 portion further comprises one or more of (i) a proline (P) residue at amino acid 148; (ii) an arginine (R) residue inserted at amino acid 152; (iii) an arginine (R) residue at amino acid 168; (iv) an isoleucine (I) residue at amino acid 189; and (v) a serine(S) residue at amino acid 200, wherein amino acid positions in the AAV capsid protein are numbered with respect to amino acid positions in the amino acid sequence of SEQ ID NO: 3.

In embodiments, one or more variable regions I through IX (see FIG. 5) in the disclosed VP3 portion capsid proteins may be removed and replaced with alternative regions. Suitable alternatives are identified in Table 6 below. The location for these, as well as the identity of additional alternatives may be identified by alignment to SEQ ID NO:41 as shown in FIG. 5. In embodiments, one or more VRs may have an insertion of 1, 2 or 3 amino acids. In embodiments, one or more VRs may have a deletion of 1, 2 or 3 amino acids.

TABLE 6
Variable Regions

| VR | Sequence |
|---|---|
| I | SASTGAS (SEQ ID NO. 52); NSTSGGSS (SEQ |
| | ID NO. 53); SSTSGGSS (SEQ ID NO: 87); |
| | NGTSGGST (SEQ ID NO: 170) |
| II | DNNGVK (SEQ ID NO. 54) |
| III | NDGS (SEQ ID NO. 55) |
| IV | INGSGQNQQT (SEQ ID NO. 56); QSTGGTAGTQQ |
| | (SEQ ID NO: 171) |
| V | RVSTTTGQNNNSNFAWTA (SEQ ID NO. 57) |
| VI | HKEGEDRFFPLSG (SEQ ID NO. 58) |
| VII | KQNAARDNADYSDV (SEQ ID NO: 59) |
| VIII | ADNLQQQNTAPQI (SEQ ID NO. 60) |
| IX | NYYKSTSVDF (SEQ ID NO. 61). |

The disclosure provides nucleic acids encoding any one of the AAV capsid proteins disclosed herein. The disclosure also provides vectors comprising any one of the nucleic acids disclosed herein.

In embodiments, AAV is an AAV9 serotype. Alternative serotypes or modified capsid viruses can be used to optimize neuronal tropism. Alternative vectors include: a modified AAV9 serotype vector for higher neuronal tropism than standard AAV9, e.g., PHP.B that uses a Cre-lox recombination system to identify neuronally targeted vectors. Alternatively, the AAV9 PHP.B has a modified amino acid 498 of VP1 from asparagine to lysine to reduce the liver tropism. Further variants of AAVrh74 that have mutated several amino acids can be used for very broad tissue tropism including the brain.

### AAV Vectors

The AAV vectors supply the nucleic acid that becomes encapsidated into the AAV vector particle including element(s) involved in controlling expression of the nucleic acids in the subject, as well as the ITRs to facilitate encapsidation. In embodiments, the AAV vectors disclosed herein comprise at least one heterologous nucleic acid (HNA) sequence, which, when expressed in a cell of a subject, is effective to treat a disease or disorder. In embodiments, the HNA sequence comprises a transgene. In embodiments, the AAV vectors comprise at least one ITR sequence and at least one transgene. In embodiments, the transgene encodes a therapeutic protein or a therapeutic RNA.

In embodiments, control of transgene expression in the host cell may be regulated by regulatory elements contained within the AAV vector, including promoter sequences, and polyadenylation signals. In embodiments, the AAV vector may also encode a signal peptide. In embodiments, the AAV vectors have 5′ and 3′ inverted terminal repeats (ITRs). The 5′ ITR is located upstream of a promoter, which in turn is upstream of the transgene. In embodiments, the 5′ and 3′ ITR have the same sequence. In embodiments, they have a different sequence. In embodiments, an AAV vector of the disclosure may comprise, in 5′ to 3′ orientation, a first (5′) ITR, a promoter, a transgene, a polyadenylation signal, and a second (3′) ITR.

In embodiments, the 5′ ITR comprises, consists essentially of, or consists of a nucleic acid sequence having at least 90%, at least 95%, at least 97%, at least 98%, at least 99%, or 100% identity to SEQ ID NO: 253. In embodiments, the 3′ ITR comprises, consists essentially of, or consists of a nucleic acid sequence having at least 90%, at least 95%, at least 97%, at least 98%, at least 99%, or 100% identity to SEQ ID NO: 254. In embodiments, the corresponding AAV vector is for expression of Opa1 transgene.

In embodiments, the 5′ ITR comprises, consists essentially of, or consists of a nucleic acid sequence having at least 90%, at least 95%, at least 97%, at least 98%, at least 99%, or 100% identity to SEQ ID NO: 255. In embodiments, the 3′ ITR comprises, consists essentially of, or consists of a nucleic acid sequence having at least 90%, at least 95%, at least 97%, at least 98%, at least 99%, or 100% identity to SEQ ID NO: 256. In embodiments, the corresponding AAV vector is for expression of RS1 transgene.

Further descriptions of ITRs can be found, for example, in McCarty et al., Gene Ther. 2003 December; 10 (26): 2112-8, the content of which is incorporated herein in its entirety.

In embodiments, the HNA (for example, an HNA comprising a transgene) is operably linked to a promoter.

In embodiments, the HNA is operably linked to a constitutive promoter. The constitutive promoter can be any constitutive promoter known in the art and/or provided herein. In embodiments, the constitutive promoter comprises, consists essentially of, or consists of a Rous sarcoma virus (RSV) LTR promoter (optionally with the RSV enhancer), a cytomegalovirus (CMV) promoter, an SV40 promoter, a dihydrofolate reductase promoter, a beta-actin promoter, a phosphoglycerol kinase (PGK) promoter, a U6 promoter, an H1 promoter, a hybrid chicken beta actin promoter, a MeCP2 promotor, an H1 promoter, a U1a promoter, a mMeP418 promoter, a mMeP426 promoter, a minimal MeCP2 promoter, a CAG promoter, or an EF1 promoter. It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). In embodiments, the HNA sequence is operably linked to a tissue-specific control promoter, or an inducible promoter. In embodiments, the tissue-specific control promoter is a central nervous system (CNS) cell-specific promoter, a lung-specific promoter, a skin-specific promoter, a muscle-specific promoter, a liver-specific promoter, an eye-specific promoter (e.g., a VMD2, or mRho promoter).

In embodiments, the promoter may comprise, consist essentially of or consist of a polynucleotide having the sequence of SEQ ID NO: 96 (mouse U1 promoter) or a SEQ ID NO: 97 (a H1 promoter). In embodiments, the promoter is an U1a or U1b promoter, EF1 promoter, or CBA (chicken beta-actin). In embodiments, the promoter may comprise, consist essentially of or consist of any one of the nucleic acid sequences listed in Table 5, or a sequence having up to 5, up to 10, up to 20, or up to 30 nucleotide changes to any one of the nucleic acid sequences listed in Table 5. In embodiments, the promoter may comprise, consist essentially of, or consist of a nucleic acid sequence having at least 90%, at least 95%, at least 97%, at least 98%, at least 99%, or 100% identity to any one of the nucleic acid sequences listed in Table 5.

In embodiments, the promoter is a Chicken beta-Actin hybrid (CBh) promoter. In embodiments, the CBh promoter comprises, consists essentially of, or consists of a nucleic acid sequence having at least 90%, at least 95%, at least 97%, at least 98%, at least 99%, or 100% identity to SEQ ID NO: 154.

In embodiments, the promoter is a Rhodopsin Kinase (RK) promoter. In embodiments, the RK promoter comprises, consists essentially of, or consists of a nucleic acid sequence having at least 90%, at least 95%, at least 97%, at least 98%, at least 99%, or 100% identity to SEQ ID NO: 196.

In embodiments, the promoter is a Rho promoter. In embodiments, the Rho promoter comprises, consists essentially of, or consists of a nucleic acid sequence having at least 90%, at least 95%, at least 97%, at least 98%, at least 99%, or 100% identity to SEQ ID NO: 197.

In embodiments, the promoter is a PDE promoter. In embodiments, the PDE promoter comprises, consists essentially of, or consists of a nucleic acid sequence having at least 90%, at least 95%, at least 97%, at least 98%, at least 99%, or 100% identity to SEQ ID NO: 198.

TABLE 5
Non-limiting Examples of Promoters

| | Nucleic acid |
|---|---|
| Promoter name | SEQ ID No. |
| Mouse U1a promoter | 152 |
| Polymerase III H1 mutant promoter | 153 |
| Chicken β-actin hybrid promoter CBh (CBh promoter | 154 |
| consists of CMV enhancer, CBA promoter, and first CBA | |
| exon) | |
| MeCP2 min promoter sequence | 155 |
| MeCP2 promoter sequence | 156 |
| MeCP418 promoter sequence | 157 |
| MeCP426 promoter sequence | 158 |
| VMD2 promoter | 159 |
| PDE6b promoter | 160 |
| mRho promoter | 161 |
| CMV promoter | 162 |
| UbC promoter | 163 |
| RK promoter | 196 |
| Rho promoter | 197 |
| PDE promoter | 198 |

In embodiments, the AAV vector comprises an enhancer. In embodiments, the enhancer is operably linked to the HNA sequence. In embodiments, the enhancer is located upstream of the promoter. In embodiments, the enhancer is located immediately upstream of the promoter, without any additional nucleotide in between.

In embodiments, the enhancer is an interphotoreceptor retinoid-binding protein (IRBP) enhancer. In embodiments, the IRBP enhancer comprises, consists essentially of, or consists of a nucleic acid sequence having at least 90%, at least 95%, at least 97%, at least 98%, at least 99%, or 100% identity to SEQ ID NO: 199.

In embodiments, the HNA sequence is operably linked to an additional regulatory element. The additional regulatory element can be a woodchuck hepatitis virus post-transcriptional regulatory element (“WPRE”). In embodiments, the AAV vector may comprise regulatory components suitable for growth and culture of the vector in a bacterial host for vector production purposes. For example, the vector may comprise genes for antibiotic resistance, and maintenance of the plasmid in bacteria, as well as associated regulatory elements to control protein expression in bacteria.

In embodiments, the HNA sequence is operably linked to a polyadenylation signal. In embodiments, the polyadenylation signal comprises, consists essentially of or consists of an MeCP2 polyadenylation signal, a retinol dehydrogenase 1 (RDH1) polyadenylation signal, a bovine growth hormone (BGH) polyadenylation signal, an SV40 polyadenylation signal, a SPA49 polyadenylation signal, a sNRP-TK65 polyadenylation signal, a sNRP polyadenylation signal, or a TK65 polyadenylation signal. An exemplary SPA49 polyadenylation signal is described in Ostedgaard et al., Proc. Nat'l Acad. Sci. USA (Feb. 22, 2005) 102:2952-2957, incorporated herein by reference. In embodiments, the polyadenylation signal sequence comprises, consists essentially of, or consists of a nucleic acid sequence having at least 90%, at least 95%, at least 97%, at least 98%, at least 99%, or 100% identity to SEQ ID NO: 201. In embodiments, the polyadenylation signal sequence comprises, consists essentially of, or consists of a nucleic acid sequence having at least 90%, at least 95%, at least 97%, at least 98%, at least 99%, or 100% identity to SEQ ID NO: 225.

In embodiments, an intron is inserted between the promoter and the HNA. In embodiments, the intron comprises, consists essentially of, or consists of a nucleic acid sequence having at least 90%, at least 95%, at least 97%, at least 98%, at least 99%, or 100% identity to SEQ ID NO: 200. In embodiments, the intron comprises, consists essentially of, or consists of a nucleic acid sequence having at least 90%, at least 95%, at least 97%, at least 98%, at least 99%, or 100% identity to SEQ ID NO: 222. In embodiments, the intron comprises, consists essentially of, or consists of a nucleic acid sequence having at least 90%, at least 95%, at least 97%, at least 98%, at least 99%, or 100% identity to SEQ ID NO: 227. In embodiments, the AAV vector genome comprises a CBA sequence located immediately upstream of the intron sequence without any additional nucleotides in between, and wherein the CBA sequence comprises, consists essentially of, or consists of the nucleic acid sequence SEQ ID NO: 229, or a sequence having at most 10, at most 9, at most 8, at most 7, at most 6, at most 5, at most 4, at most 3, at most 2, or at most 1 mutation(s) thereto. In embodiments, the intron comprises, consists essentially of, or consists of a nucleic acid sequence having at least 90%, at least 95%, at least 97%, at least 98%, at least 99%, or 100% identity to SEQ ID NO: 226. In embodiments, the intron does not comprise polynucleotide sequence “ATG”.

In embodiments, a first telomeric repeat is inserted between the polyadenylation signal and the 3′ ITR. In embodiments, the telomeric repeat comprises a repeat unit of CCCTAA (SEQ ID NO: 217). In embodiments, the telomeric repeat comprises an intermediate repeat comprising 1, 2, 3, 4, 5, 6, 7, 8, 9, 10, or more, consecutive copies of the repeat unit of CCCTAA (SEQ ID NO: 217). In embodiments, the telomeric repeat comprises 1, 2, 3, 4, 5, 6, 7, 8, 9, 10, or more, copies of the intermediate repeat. In embodiments, the copies of the intermediate repeat is separated by a spacer comprising TTTTT (SEQ ID NO: 218). In embodiments, the first telomeric repeat comprises, consists essentially of, or consists of a nucleic acid sequence having at least 90%, at least 95%, at least 97%, at least 98%, at least 99%, or 100% identity to SEQ ID NO: 202.

In embodiments, a first telomeric repeat is inserted between the polyadenylation signal and the 3′ ITR. In embodiments, the telomeric repeat comprises a repeat unit of TTAGGG (SEQ ID NO: 219). In embodiments, the telomeric repeat comprises an intermediate repeat comprising 1, 2, 3, 4, 5, 6, 7, 8, 9, 10, or more, consecutive copies of the repeat unit of TTAGGG (SEQ ID NO: 219). In embodiments, the telomeric repeat comprises 1, 2, 3, 4, 5, 6, 7, 8, 9, 10, or more, copies of the intermediate repeat. In embodiments, the copies of the intermediate repeat is separated by a spacer comprising AAAAA (SEQ ID NO: 220). In embodiments, the first telomeric repeat comprises, consists essentially of, or consists of a nucleic acid sequence having at least 90%, at least 95%, at least 97%, at least 98%, at least 99%, or 100% identity to SEQ ID NO: 203.

In embodiments, a second telomeric repeat is inserted between the 5′ ITR and the promoter. In embodiments, the telomeric repeat comprises a repeat unit of TTAGGG (SEQ ID NO: 219). In embodiments, the telomeric repeat comprises an intermediate repeat comprising 1, 2, 3, 4, 5, 6, 7, 8, 9, 10, or more, consecutive copies of the repeat unit of TTAGGG (SEQ ID NO: 219). In embodiments, the telomeric repeat comprises 1, 2, 3, 4, 5, 6, 7, 8, 9, 10, or more, copies of the intermediate repeat. In embodiments, the copies of the intermediate repeat is separated by a spacer comprising AAAAA (SEQ ID NO: 220). In embodiments, the second telomeric repeat comprises, consists essentially of, or consists of a nucleic acid sequence having at least 90%, at least 95%, at least 97%, at least 98%, at least 99%, or 100% identity to SEQ ID NO: 203.

In embodiments, a human beta-globin scaffold/matrix attachment region (βGlo_s/MAR) sequence is inserted between the polyadenylation signal and the downstream telomeric repeat, or is inserted between the polyadenylation signal and the downstream 3′ ITR. In embodiments, the βGlo_s/MAR sequence comprises, consists essentially of, or consists of a nucleic acid sequence having at least 90%, at least 95%, at least 97%, at least 98%, at least 99%, or 100% identity to SEQ ID NO: 221.

### Heterologous Nucleic Acids (HNA)

The AAV viral vectors disclosed herein infect and deliver one or more heterologous nucleic acids (HNA) to target tissues. In embodiments, the HNA sequences are transcribed and optionally, translated in the cells of the target tissue.

In some cases, the HNA encodes an antisense RNA, microRNA, siRNA, or guide RNA (gRNA). CRISPR technology has been used to target the genome of living cells for modification. Cas9 protein is a large enzyme that must be delivered efficiently to target tissues and cells to mediate gene repair through the CRISPR system and current CRISPR/Cas9 gene correction protocols suffer from a number of drawbacks. Long-term expression of Cas9 can elicit host immune responses. An additional guide RNA may be delivered via a separate vector due to packaging constraints. In embodiments, the HNA encodes a Cas9 protein or an equivalent thereof.

In embodiments, the HNA comprises a transgene encoding a protein, which may be expressed in cells of a subject to treat a disease or a disorder, resulting from reduced or eliminated activity of the native protein. Thus, in embodiments, the transgene may encode a protein selected from cystic fibrosis transmembrane conductance regulator (CFTR), N-acetyl-alpha-glucosaminidase (NAGLU), N-sulfoglucosamine sulfohydrolase (SGSH), palmitoyl-protein thioesterase 1 (PPT1), survival of motor neuron 1, telomeric (SMN1), alkaline phosphatase, biomineralization associated (ALPL, also known as TNALP), glial cell derived neurotrophic factor (GDNF), glucosylceramidase beta (GBA1), iduronidase alpha-L-(IDUA), methyl-CpG binding protein 2 (MeCP2), ceroid lipofuscinosis, neuronal, 1 (CLN1), rhodopsin (Rho), cytochrome P450 family 4 subfamily V member 2 (CYP4V2), retinoschisin 1 (RS1), phosphodiesterase 6B (PDE6B), ATP binding cassette subfamily A member 4 (ABCA4), Bestrophin-1 (BEST1), OPA1 Mitochondrial Dynamin Like GTPase (OPA1), and Optic Atrophy 3 (OPA3).

In embodiments, the transgene encodes a Cytochrome P450 family 4 subfamily V member 2 (CYP4V2). In embodiments, the CYP4V2 comprises a mutant sequence, a codon-optimized sequence, and/or a truncated sequence of CYP4V2. In embodiments, the CYP4V2 comprises, consists essentially of, or consists of a nucleic acid having the sequence of SEQ ID NO: 116, or a sequence having at least 80%, at least 85%, at least 90%, at least 95%, at least 97%, at least 98%, at least 99%, or at least 99.5% identity to SEQ ID NO: 116. In embodiments, the CYP4V2 encodes a protein that comprises, consists essentially of, or consists of an amino acid sequence of SEQ ID NO: 142, or a sequence having at least 80%, at least 85%, at least 90%, at least 95%, at least 97%, at least 98%, at least 99%, or at least 99.5% identity to SEQ ID NO: 142. In embodiments, the AAV vector or AAV vector genome of the disclosure encodes CYP4V2 and is for treating Bietti's Crystalline Dystrophy.

In embodiments, the transgene encodes a Retinoschisin 1 (RS1). In embodiments, the RS1 transgene comprises a mutant sequence, a codon-optimized sequence, and/or a truncated sequence of RS1. In embodiments, the RS1 comprises, consists essentially of, or consists of a nucleic acid having the sequence of SEQ ID NO: 117, or a sequence having at least 80%, at least 85%, at least 90%, at least 95%, at least 97%, at least 98%, at least 99%, or at least 99.5% identity to SEQ ID NO: 117. In embodiments, the RS1 encodes a protein that comprises, consists essentially of, or consists of an amino acid sequence of SEQ ID NO: 143, or a sequence having at least 80%, at least 85%, at least 90%, at least 95%, at least 97%, at least 98%, at least 99%, or at least 99.5% identity to SEQ ID NO: 143. In embodiments, the AAV vector or AAV vector genome of the disclosure encodes RS1 and is for treating Retinoschisis.

In embodiments, the transgene encodes a Phosphodiesterase 6B (PDE6B). In embodiments, the PDE6B transgene comprises a mutant sequence, a codon-optimized sequence, and/or a truncated sequence of PDE6B. In embodiments, the RS1 comprises, consists essentially of, or consists of a nucleic acid having the sequence of SEQ ID NO: 118, or a sequence having at least 80%, at least 85%, at least 90%, at least 95%, at least 97%, at least 98%, at least 99%, or at least 99.5% identity to SEQ ID NO: 118. In embodiments, the PDE6B encodes a protein that comprises, consists essentially of, or consists of an amino acid sequence of SEQ ID NO: 144, or a sequence having at least 80%, at least 85%, at least 90%, at least 95%, at least 97%, at least 98%, at least 99%, or at least 99.5% identity to SEQ ID NO: 144. In embodiments, the AAV vector or AAV vector genome of the disclosure encodes PDE6B and is for treating Retinitis pigmentosa.

In embodiments, the transgene encodes an ATP binding cassette subfamily A member 4 (ABCA4). In embodiments, the ABCA4 transgene comprises a mutant sequence, a codon-optimized sequence, and/or a truncated sequence of ABCA4. In embodiments, the ABCA4 comprises, consists essentially of, or consists of a nucleic acid having the sequence of SEQ ID NO: 172, or a sequence having at least 80%, at least 85%, at least 90%, at least 95%, at least 97%, at least 98%, at least 99%, or at least 99.5% identity to SEQ ID NO: 172. In embodiments, the ABCA4 encodes a protein that comprises, consists essentially of, or consists of an amino acid sequence of SEQ ID NO: 177, or a sequence having at least 80%, at least 85%, at least 90%, at least 95%, at least 97%, at least 98%, at least 99%, or at least 99.5% identity to SEQ ID NO: 177. In embodiments, the AAV vector or AAV vector genome of the disclosure encodes ABCA4 and is for treating Stargardt disease.

In embodiments, the transgene encodes a Bestrophin-1 (BEST1). In embodiments, the BEST1 transgene comprises a mutant sequence, a codon-optimized sequence, and/or a truncated sequence of BEST1. In embodiments, the BEST1 comprises, consists essentially of, or consists of a nucleic acid having the sequence of SEQ ID NO: 173 or 174, or a sequence having at least 80%, at least 85%, at least 90%, at least 95%, at least 97%, at least 98%, at least 99%, or at least 99.5% identity to SEQ ID NO: 173 or 174. In embodiments, the BEST1 encodes a protein that comprises, consists essentially of, or consists of an amino acid sequence of SEQ ID NO: 178 or 179, or a sequence having at least 80%, at least 85%, at least 90%, at least 95%, at least 97%, at least 98%, at least 99%, or at least 99.5% identity to SEQ ID NO: 178 or 179. In embodiments, the AAV vector or AAV vector genome of the disclosure encodes BEST1 and is for treating BEST vitelliform macular dystrophy.

In embodiments, the transgene encodes an OPA1 Mitochondrial Dynamin Like GTPase (OPA1). In embodiments, the OPA1 transgene comprises a mutant sequence, a codon-optimized sequence, and/or a truncated sequence of OPA1. In embodiments, the OPA1 transgene comprises, consists essentially of, or consists of a nucleic acid having the sequence of SEQ ID NO: 175, or a sequence having at least 80%, at least 85%, at least 90%, at least 95%, at least 97%, at least 98%, at least 99%, or at least 99.5% identity to SEQ ID NO: 175. In embodiments, the OPA1 transgene encodes a protein that comprises, consists essentially of, or consists of an amino acid sequence of SEQ ID NO: 180, or a sequence having at least 80%, at least 85%, at least 90%, at least 95%, at least 97%, at least 98%, at least 99%, or at least 99.5% identity to SEQ ID NO: 180. In embodiments, the OPA1 transgene is a DeltaS1 (ΔS1) isoform, which comprises, consists essentially of, or consists of a nucleic acid having the sequence of SEQ ID NO: 182, or a sequence having at least 80%, at least 85%, at least 90%, at least 95%, at least 97%, at least 98%, at least 99%, or at least 99.5% identity to SEQ ID NO: 182. In embodiments, the OPA1 transgene is a DeltaS1 isoform, which encodes a protein that comprises, consists essentially of, or consists of an amino acid sequence of SEQ ID NO: 183, or a sequence having at least 80%, at least 85%, at least 90%, at least 95%, at least 97%, at least 98%, at least 99%, or at least 99.5% identity to SEQ ID NO: 183. In embodiments, the OPA1 transgene is an E5b isoform, which comprises, consists essentially of, or consists of a nucleic acid having the sequence of SEQ ID NO: 184, or a sequence having at least 80%, at least 85%, at least 90%, at least 95%, at least 97%, at least 98%, at least 99%, or at least 99.5% identity to SEQ ID NO: 184. In embodiments, the OPA1 is an E5b isoform of the transgene, which encodes a protein that comprises, consists essentially of, or consists of an amino acid sequence of SEQ ID NO: 185, or a sequence having at least 80%, at least 85%, at least 90%, at least 95%, at least 97%, at least 98%, at least 99%, or at least 99.5% identity to SEQ ID NO: 185. In embodiments, the AAV vector or AAV vector genome of the disclosure encodes OPA1 and is for treating Dominant Optic Atrophy.

In embodiments, the transgene encodes an Optic Atrophy 3 (OPA3). In embodiments, the OPA3 transgene comprises a mutant sequence, a codon-optimized sequence, and/or a truncated sequence of OPA3. In embodiments, the OPA3 comprises, consists essentially of, or consists of a nucleic acid having the sequence of SEQ ID NO: 176, or a sequence having at least 80%, at least 85%, at least 90%, at least 95%, at least 97%, at least 98%, at least 99%, or at least 99.5% identity to SEQ ID NO: 176. In embodiments, the OPA3 encodes a protein that comprises, consists essentially of, or consists of an amino acid sequence of SEQ ID NO: 181, or a sequence having at least 80%, at least 85%, at least 90%, at least 95%, at least 97%, at least 98%, at least 99%, or at least 99.5% identity to SEQ ID NO: 181. In embodiments, the AAV vector or AAV vector genome of the disclosure encodes OPA3 and is for treating Dominant Optic Atrophy.

In embodiments, the transgene comprises any one of the nucleic acid sequences listed in Table 4, or a sequence having up to 5, up to 10, or up to 30 nucleotide changes to any one of the DNA sequences in Table 4 (SEQ ID NOs: 116-118 and 172-176). In embodiments, the transgene encodes any one of the amino acid sequences listed in Table 4, or a sequence having up to 1, 2, 3, 4, 5, 6, 7, 8, 9, or 10 amino acids different from any one of the amino acid sequences listed in Table 4 (SEQ ID NOs: 142-144 and 177-118).

TABLE 4
Non-limiting Examples of Transgenes

| | | Amino acid SEQ | |
|---|---|---|---|
| | Nucleic acid | ID Nos. encoded | |
| Name of transgene | SEQ ID Nos. | by transgene | Features |
| Cytochrome P450 family | 116 | 142 | Natural |
| 4 subfamily V member 2 | | | |
| (CYP4V2) | | | |
| Retinoschisin 1 (RS1) | 117 | 143 | Natural |
| Phosphodiesterase 6B | 118 | 144 | Natural |
| (PDE6B) | | | |
| ATP binding cassette | 172 | 177 | Natural |
| subfamily A member 4 | | | |
| (ABCA4) | | | |
| Bestrophin-1 (BEST1) | 173 | 178 | Natural |
| isoform 1 | | | |
| Bestrophin-1 (BEST1) | 174 | 179 | Natural |
| isoform 2 | | | |
| OPA1 Mitochondrial | 175 | 180 | Natural |
| Dynamin Like GTPase | 182 | 183 | Natural (ΔS1 |
| (OPA1) | | | isoform) |
| | 184 | 185 | Natural (E5b |
| | | | isoform) |
| Optic Atrophy 3 | 176 | 181 | Natural |
| (OPA3) | | | |

In embodiments, the transgene comprises a nucleic acid sequence set forth in any one of SEQ ID NOs: 99-133 and 172-176, or a sequence having up to 5, up to 10, or up to 30 nucleotide changes to any one of SEQ ID NOs: 99-133 and 172-176. In embodiments, the transgene encodes an amino acid sequence set forth in any one of SEQ ID NOs: 134-151 and 177-181, or a sequence having up to 1, 2, 3, 4, 5, 6, 7, 8, 9, or 10 amino acids different from any one of the amino acid sequences SEQ ID NOs: 134-151 and 177-181.

In embodiments, the heterologous nucleic acid encodes a reporter protein; for example, a fluorescent protein.

### Methods of Producing AAV Viral Vectors

A variety of approaches may be used to produce AAV viral vectors. In embodiments, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In embodiments, the cell is a packaging or helper cell line. In embodiments, the helper cell line is eukaryotic cell; for example, an HEK 293 cell or 293T cell. In embodiments, the helper cell is a yeast cell or an insect cell.

In embodiments, the cell comprises a nucleic acid encoding a tetracycline activator protein; and a promoter that regulates expression of the tetracycline activator protein. In embodiments, the promoter that regulates expression of the tetracycline activator protein is a constitutive promoter. In embodiments, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing AAV particles comprising transfecting a packaging cell line with any one of the AAV helper plasmids disclosed herein; and any one of the AAV vectors disclosed herein. In embodiments, the AAV helper plasmid and the AAV vector are co-transfected into the packaging cell line. In embodiments, the cell line is a mammalian cell line, for example, human embryonic kidney (HEK) 293 cell line. The disclosure provides cells comprising any one of the AAV vectors and/or AAV particles disclosed herein.

### Pharmaceutical Compositions

The disclosure provides pharmaceutical compositions comprising any one of the AAV vectors, AAV capsids and/or AAV particles described herein. Typically, the AAV particles are administered for therapy.

The pharmaceutical composition, as described herein, may be formulated by any methods known or developed in the art of pharmacology, which include but are not limited to contacting the active ingredients (e.g., viral particles or AAV vectors) with an excipient or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with AAV vectors or transduced with AAV viral particles (e.g., for transplantation into a subject), nanoparticle mimics or combinations thereof. In embodiments, the pharmaceutical composition is formulated as a nanoparticle. In embodiments, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients, each in an amount that together increases the stability of the viral vector, increases cell transfection or transduction by the viral vector, increases the expression of viral vector encoded protein, and/or alters the release profile of viral vector encoded proteins. In embodiments, the pharmaceutical composition comprises an excipient. Non limiting examples of excipients include solvents, dispersion media, diluents, or other liquid vehicles, dispersion or suspension aids, surface active agents, isotonic agents, thickening or emulsifying agents, preservatives, or combination thereof.

In embodiments, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

### Therapeutic Methods

This disclosure provides methods of preventing or treating a disorder, comprising, consisting essentially of, or consisting of administering to a subject a therapeutically effective amount of any one of the pharmaceutical compositions disclosed herein.

In embodiments, the disorder is a CNS disorder, a skin disorder, a lung disorder, a muscle disorder, a liver disorder, or an ophthalmic disease (or a retinal disease). In embodiments, the disorder is cystic fibrosis. In embodiments, the disorder is an ophthalmic disease. In embodiments, the disorder is a retinal disease.

In embodiments, the disorder is hypophosphatasia, amyotrophic lateral sclerosis (ALS), spinal muscular atrophy (SMA), recessive dystrophic epidermolysis bullosa (RDEB), lysosomal storage disorder (including Duchenne's Muscular Dystrophy, and Becker muscular dystrophy), juvenile Batten disease, infantile Batten disease, autosomal dominant disorders, muscular dystrophy, Bietti's Crystalline Dystrophy, retinoschisis (e.g., degenerative, hereditary, tractional, exudative), hemophilia A, hemophilia B, multiple sclerosis, diabetes mellitus, Fabry disease, Pompe disease, neuronal ceroid lipofuscinosis 1 (CLN1), CLN3 disease (or Juvenile Neuronal Ceroid Lipofuscinosis), Gaucher disease, cancer, arthritis, muscle wasting, heart disease, intimal hyperplasia, Rett syndrome, epilepsy, Huntington's disease, Parkinson's disease, Alzheimer's disease, an autoimmune disease, cystic fibrosis, thalassemia, Hurler's Syndrome (MPS IH), Sly syndrome, Scheie Syndrome, Hurler-Scheie Syndrome, Hunter's Syndrome, Sanfilippo Syndrome A (mucopolysaccharidosis IIIA or MPS IIIA), Sanfilippo Syndrome B (mucopolysaccharidosis IIIB or MPS IIIB), Sanfilippo Syndrome C, Sanfilippo Syndrome D, Morquio Syndrome, Maroteaux-Lamy Syndrome, Krabbe's disease, phenylketonuria, spinal cerebral ataxia, LDL receptor deficiency, hyperammonemia, anemia, arthritis, or adenosine deaminase deficiency.

In embodiments, the disorder is X-linked retinoschisis (XLRS), a rare, monogenic disease that results in severe visual impairment. While female carriers are asymptomatic, affected males usually begin exhibiting disease symptoms within the first decade, and occasionally during infancy. The disease results from mutations in the RS1 gene, which is expressed in photoreceptors and retinal bipolar cells. The gene product is a secreted protein that localizes primarily to the inner segment of photoreceptor cells, and more diffusely to the rest of the neural retina. RS1 forms a homo-octameric complex and is thought to mediate cell-cell adhesion via interactions with extracellular epitopes of membrane proteins. In individuals with XLRS, cavities develop where the adhesion of adjacent retinal layers is disrupted, typically in the outer plexiform layer where photoreceptors synapse with inner retinal neurons. This leads to discontinuity within the retinal circuitry, photoreceptor degeneration, and impaired visual acuity. Many of the RS1 mutations that have been reported in the literature are point mutations that are predicted to disrupt secretion of the protein and are therefore functionally equivalent to a null allele. The current standard of care for XLRS patients is palliative and involves correction of refractive errors, low-vision aids, and genetic counseling. Complications such as retinal detachment (up to 22% of patients) and vitreal hemorrhage (up to 40% of patients) are most frequent in the later stages of disease and can be treated surgically. Early intervention via gene therapy has significant potential to reverse or stabilize disease progression at early stages of disease and prevent significant vision loss as well as the occurrence of these more severe complications.

In embodiments, the disorder is Autosomal Dominant Optic Atrophy (ADOA). ADOA is caused by Opa1 mutations, resulting in vision loss in the second to third decade of life. In embodiments, Opa1 homozygous mutants are embryonic lethal and therefore do not survive past E9-12. Heterozygous (HT) animals survive to term but have retinal degeneration, neurological defects, and musculoskeletal complications over time. Mice show Optic nerve atrophy upon fundus and Scanning Laser Ophthalmoscopy (SLO) exams well as decreased Electroretinogram (ERG) amplitudes, and fibrosis in the inner limiting membrane (ILM), and retinal nerve fiber layer (RNFL). Opa1 has been shown to be involved in mitochondrial christae structure, mitochondrial fusion, and mitochondrial inner membrane remodeling.

In embodiments, the disclosure provides methods of expressing a transgene in a retinal cell. In embodiments, the method comprises delivering a nucleic acid of the disclosure to the retinal cell. In embodiments, the method comprises transducing the retinal cell with the AAV viral vector of the disclosure.

In embodiments, the target cells of the disclosure comprise retinal cells. In embodiments, the retinal cells comprise a photoreceptor, a bipolar cell, a retinal ganglion cell, a horizontal cell, or an amacrine cell. In embodiments, the retinal cells comprise a retinal ganglion cell. In embodiments, the retinal cells comprise a bipolar cell. In embodiments, the retinal cells comprise a horizontal cell. In embodiments, the retinal cells comprise an amacrine cell. In embodiments, the retinal cells comprise a photoreceptor. In embodiments, the photoreceptor comprises a rod cell and/or a cone cell.

In embodiments, the target cells of the disclosure comprise, consist essentially of, or consist of photoreceptor cells. In embodiments, the transgene of the disclosure is operably linked to a RK promoter for selective expression in photoreceptor cells.

In addition to specific transgenes disclosed herein, known active enzyme sequences may be used as transgenes to deliver functional enzyme activity.

In embodiments, the disorder is CLN3 disease. CLN3 disease or Juvenile Neuronal Ceroid Lipofuscinosis is a lysosomal storage disease caused by an autosomal recessively inherited mutation in the CLN3 gene. CLN3 disease is a progressive neurodegenerative disorder in which the central nervous system (CNS) is greatly affected resulting in behavioral issues, vision loss, and other cognitive disabilities.

In embodiments, the disorder is Fabry disease. Fabry disease is an X-linked lysosomal storage disorder caused by a deficiency in alpha-galactosidase A (GLA) activity that results in the accumulation of the glycolipid products, globotriaosylceramide (Gb3) and lyso-Gb3 in the lysosome. Disease presentation is highly heterogeneous but usually includes frequent bouts of peripheral neurotrophic pain, angiokeratomas, reduced sweat production, corneal dystrophy, and gastrointestinal complications. As the disease progresses patients suffer from cardiomyopathy, renal insufficiency and cerebrovascular disease, all of which are the primary causes of reduced life-span in Fabry patients. While males are the most severely affected population of patients with mutations in the GLA gene, it has become increasingly clear that female patients are also frequently symptomatic but are often misdiagnosed. Enzyme replacement therapy (ERT) is currently the only FDA-approved therapy to treat Fabry and requires bi-weekly injections of relatively large quantities of recombinant protein. While ERT reduces the accumulation of Gb3 in the heart, kidney and vasculature it fails to completely treat all symptoms of Fabry, primarily due to its inability to efficiently enter the CNS. Gene therapy strategies have been investigated and while many show great promise in correcting the glycolipid accumulation, most have failed to efficiently enter the CNS and also suffered from an immune response often seen during GLA replacement.

In embodiments, the AAV viral vectors disclosed herein are used to treat Fabry disease in patients, who are unresponsive to ERT, or when ERT fails to address all symptoms. In embodiments, the AAV viral vectors disclosed herein are used to treat Fabry disease in patients who have already been administered ERT.

In embodiments, the disorder is Pompe disease. Pompe disease is a lysosomal storage disorder caused by a deficiency in acid α-glucosidase (GAA) activity that results in the accumulation of glycogen in the lysosome. The disease presents as a form of muscular dystrophy which primarily affects both smooth and striated musculature as well as the central nervous system (CNS), with early mortality. Enzyme replacement therapy (ERT) is currently the only FDA-approved therapy to treat Pompe and requires bi-weekly injections of relatively large quantities of recombinant protein. While ERT significantly reduces the mortality rate of infantile Pompe patients, who typically die by the age of two without therapy, it fails to completely ameliorate all symptoms of Pompe, primarily due to its inability to efficiently enter the CNS and resulting immune responses to the GAA protein. Gene therapy strategies have been investigated and while many show great promise in correcting the glycogen accumulation and other symptoms of Pompe. Most have suffered from the severe immune response seen during GAA replacement. Previous work has demonstrated that hepatic-specific expression can make animals tolerate to the GAA protein and significantly reduce the humoral response.

In embodiments, the AAV viral vectors disclosed herein are used to treat Pompe disease in patients who have already been administered ERT; for example those who are unresponsive to ERT, or when ERT fails to address all their symptoms.

In embodiments, the AAV viral vectors disclosed herein are used to treat a cancer. In embodiments, the cancer is a solid cancer; for example, bladder, breast, cervical, colon, rectal, endometrial, kidney, lip, oral, liver, melanoma, mesothelioma, non-small cell lung, non-melanoma skin, ovarian, pancreatic, prostate, sarcoma, small cell lung tumor, or thyroid.

In embodiments, the disorder is an ophthalmic disease. The eye is immune privileged tissue. Only a very small number of viruses is necessary for therapeutic benefit. In embodiments, the ophthalmic disease affects photoreceptor and RPE cells. In embodiments, the ophthalmic disease comprises, consists essentially of, or consists of retinitis pigmentosa (e.g., autosomal recessive (SPATA7 gene; LRAT gene; TULP1 gene), autosomal dominant (AIPL1 gene), and X-linked (RPGR gene)), eye disorders related to mutations in the bestrophin-1 (BEST-1 or BEST1) gene (e.g., vitelliform macular dystrophy, age-related macular degeneration, autosomal dominant vitreoretinochoroidopathy, glaucoma, cataracts), Leber congenital amaurosis (LCA; aryl-hydrocarbon interacting protein-like 1 (AIPL1) gene), cone-rod dystrophy (CRD; ABCA4 gene), Stargardt's (ABCA4 gene), choroideremia (CHM gene), Usher Syndrome (MYO7A gene; CDH23 gene; USH2A gene; CLRN1 gene), Dominant Optic Atrophy (e.g., autosomal (OPA1 gene; OPA3 gene)), retinitis pigmentosa (PDE6B gene), retinoschisis (RS1 gene), Bietti's Crystalline Dystrophy (CYP4V2 gene) or Achromatopsia (CNGA3 gene, CNGB3 gene, GNAT2 gene, PDE6C gene, or PDE6H gene).

In embodiments, the disclosure provides methods of expressing a transgene in a retinal cell. In embodiments, the method comprises delivering a nucleic acid of the disclosure to the retinal cell. In embodiments, the method comprises transducing the retinal cell with the AAV viral vector of the disclosure.

In embodiments, the target cells of the disclosure comprise retinal cells. In embodiments, the retinal cells comprise a photoreceptor, a bipolar cell, a retinal ganglion cell, a horizontal cell, or an amacrine cell. In embodiments, the retinal cells comprise a retinal ganglion cell. In embodiments, the retinal cells comprise a bipolar cell. In embodiments, the retinal cells comprise a horizontal cell. In embodiments, the retinal cells comprise an amacrine cell. In embodiments, the retinal cells comprise a photoreceptor. In embodiments, the photoreceptor comprises a rod cell and/or a cone cell.

In embodiments, the target cells of the disclosure comprise, consist essentially of, or consist of photoreceptor cells.

In In embodiments, the subject is a mammal; for example, a human. In particular aspects, the human is an infant human; for example, under 3 years old, 2 years old, or under 1 year old.

The methods of treatment and prevention disclosed herein may be combined with appropriate diagnostic techniques to identify and select patients for the therapy or prevention. For example, the method of treating or preventing a disorder disclosed herein may further comprise steps of performing a genetic test to identify a gene mutation or deletion related to the disorder in the subject. In embodiments, the method of treating or preventing a disorder comprises administering to a subject who has been previously identified as carrying a mutation related to the disorder, or as being at high risk for developing the disorder (for example, based on hereditary factors).

The disclosure provides methods of increasing the level of a protein in a host cell, comprising contacting the host cell with any one of the AAV particles disclosed herein, wherein the AAV particle comprises any one of the AAV vector genomes disclosed herein, comprising an HNA sequence encoding the protein. In embodiments, the protein is a therapeutic protein. In embodiments, the host cell is in vitro, in vivo, or ex vivo. In embodiments, the host cell is derived from a subject. In embodiments, the subject suffers from a disorder, which results in a reduced level and/or functionality of the protein, as compared to the level and/or functionality of the protein in a normal subject.

In embodiments, the level of the protein is increased to level of about 1×10⁻⁷ ng, about 3×10⁻⁷ ng, about 5×10⁻⁷ ng, about 7×10⁻⁷ ng, about 9×10⁻⁷ ng, about 1×10⁻⁶ ng, about 2×10⁻⁶ ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×10⁻⁶ ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

The disclosure provides methods of introducing a gene of interest to a cell in a subject comprising contacting the cell with an effective amount of any one of the AAV viral particles disclosed herein, wherein the AAV viral particle contains any one of the AAV vector genomes disclosed herein, comprising the gene of interest.

### Dosage and Administration

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10¹⁷ vector genomes per administration.

In embodiments, the disclosure provides methods of treating a disease or disorder in a subject in need thereof, comprises administering an effective amount of the therapeutic agent (e.g., AAV viral vector) to the subject.

In embodiments of the methods described herein, the number of viral particles (e.g., AAV) administered to the subject ranges from about 10⁹ to about 10¹⁷. In embodiments, about 10¹⁰ to about 10¹², about 10¹¹ to about 10¹³, about 10¹¹ to about 10¹², about 10¹¹ to about 10¹⁴, about 5×10¹¹ to about 5×10¹², or about 10¹² to about 10¹³ viral particles are administered to the subject. In embodiments, the amount of viral genomes (vg) administered to the subject ranges from about 10⁹ to about 10¹⁷. In embodiments, about 10¹⁰, 2×10¹⁰, 3×10¹⁰, 4×10¹⁰, 5×10¹⁰, 6×10¹⁰, 7×10¹⁰, 8×10¹⁰, 9×10¹⁰, 10¹¹, 2×10¹¹, 3×10¹¹, 4×10¹¹, 5×10¹¹, 6×10¹¹, 7×10¹¹, 8×10¹¹, 9×10¹¹, or 10¹², viral genomes (vg) are administered to the subject. In embodiments, about 10¹⁰ to about 10¹¹, about 10¹¹ to about 10¹², about 10¹² to about 10¹³ vg, about 5×10⁹ to about 5×10¹⁰, about 5×10¹⁰ to about 5×10¹¹, about 5×10¹¹ to about 5×10¹², about 10¹⁰ to about 10¹², about 10¹¹ to about 10¹³, about 10¹⁰ to about 10¹³, or about 10¹¹ to about 10¹⁴, viral genomes (vg) are administered to the subject. For administration to a human eye, a total dose of about 1×10¹⁰ vg/eye may be used, and a total dose of 5×10⁹ vg/eye may be used for a mouse eye. Non-invasive, in vivo imaging techniques can be used to monitor efficacy/safety in animals, which include but are not limited to scanning laser ophthalmoscopy (SLO), optical coherence tomography (OCT), multi-photon microscopy, fluorescein angiography.

In embodiments, the AAV particles repair the gene deficiency in a subject. In embodiments, the ratio of repaired target polynucleotide or polypeptide to unrepaired target polynucleotide or polypeptide in a successfully treated cell, tissue, organ or subject is at least about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to Western analysis, Northern analysis, Southern analysis, PCR, sequencing, mass spectrometry, flow cytometry, immunohistochemistry (IHC), immunofluorescence, fluorescence in situ hybridization, next generation sequencing, immunoblot, and ELISA.

In embodiments, the viral particle is introduced to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intrapleurally, topically, intralymphatically, intracisternally; such introduction may also be intra-arterial, intracardiac, subventricular, epidural, intracerebral, intracerebroventricular, sub-retinal, para-retinal, intravitreal, intraarticular, intraperitoneal, intrauterine, or any combination thereof. In embodiments, the viral particles are delivered to a desired target tissue, e.g., to the lung, eye, or CNS, as non-limiting examples. In embodiments, delivery of viral particles is systemic. The intracisternal route of administration involves administration of a drug directly into the cerebrospinal fluid of the brain ventricles. It could be performed by direct injection into the cisterna magna or via a permanently positioned tube.

For treating an ophthalmic disease (or an eye disorder) intraocularly, there are multiple modes of administration known to those skilled in the art, including but not limited to: lacrimal gland (LG) administration, topical eye drop, intra-stromal administration to the cornea, intra-cameral administration (anterior chamber), intravitreal administration, sub-retinal administration, para-retinal administration, systemic administration, or a combination thereof. 80% of genetic eye disorders occur in the photoreceptors. Intravitreal delivery of small volume gene therapies can occur in an out-patient clinic.

In embodiments, the mode of administration is para-retinal administration. As used herein, the term “para-retinal administration” refers to a form of intravitreal administration that injects the therapeutic agent (e.g., an AAV viral vector) into the vitreous cavity in close proximity to the desired region of the retina (i.e., targeted delivery). In embodiments, the desired region of the retina is near the fovea area of the retina. In contrast to routine intravitreal administrations, which are done using short needles designed to deposit product in the mid-vitreous cavity, and do not require direct visualization, para-retinal injection is done under direct visualization of a longer needle capable of delivering product in the posterior vitreous cavity close to the retina. In embodiments, the therapeutic agent is deposited in the vitreous cavity at a distance of 0 mm to 13 mm from the surface of the retina, at a distance of 0 mm to 10 mm from the surface of the retina, at a distance of 0 mm to 5 mm from the surface of the retina, or at a distance of 0 mm to 3 mm from the surface of the retina. In embodiments, the therapeutic agent is deposited in the vitreous cavity at a distance of between 0-13 mm, between 0-12 mm, between 0-11 mm, between 0-10 mm, between 0-9 mm, between 0-8 mm, between 0-7 mm, between 0-6 mm, between 0-5 mm, between 0-4 mm, between 0-3 mm, between 0-2 mm, or between 0-1 mm, from the surface of the retina.

In embodiments, para-retinal administration is used in situations in which sub-retinal injection is not appropriate. In embodiments, para-retinal administration is used for targeted transduction of optic nerves. In embodiments, para-retinal administration is used for treating diseases or disorders that are related to dysfunction of the optic nerves. In embodiments, para-retinal administration is used for treating Dominant Optic Atrophy or Retinoschisis.

In embodiments, para-retinal administration involves the use of a small gauge needle (30 gauge or similar) with length sufficient to reach the posterior pole of the human eye (25 mm or similar), exo- or endo-illumination and visualization using a microscope, and use of a corneal contact lens to allow focus on the posterior vitreous cavity and retina. This is typically conducted after adequate analgesia and antisepsis, at which time the corneal contact lens is coupled to the eye and the microscope is positioned to view the posterior retina. The needle is inserted through the eye wall in the pars plana region and its tip is visualized. Under direct visualization, the needle tip is advanced to the desired location close to the retinal surface. The syringe plunger is advanced to slowly deposit the viral vector (which may be contained in any suitable composition or formulation). The needle is withdrawn and the eye is inspected. The port may be closed with a suture, but for a sufficiently small-caliber needle (such as 30 gauge), no suture is needed to close the needle tract. Ointment and an eye shield may be applied and, if desired, the subject can be kept in a supine position for a period post-operatively to further facilitate a high para-retinal concentration of the product. Variations on delivery instrumentation can include creation of a sclerotomy with or without the use of a vitrectomy port to allow use of a blunt cannula, and/or a cannula design with a tapered and/or flexible extendable tip or side ports to optimize proximity to the retinal surface and safety, and/or use of a pneumatic system in lieu of a simple syringe plunger. Additional descriptions of para-retinal administration are disclosed, for example, in WO 2020/018766 and Zeng et al., Mol Ther Methods Clin Dev. 2020 Sep. 11; 18:422-427, the contents of each of which are incorporated herein by reference in their entireties for all purposes.

In embodiments, the mode of administration is sub-retinal administration, which injects the materials into the sub-retinal space between retinal pigment epithelium (RPE) cells and photoreceptors. In the sub-retinal space, the injected materials come into direct contact with the plasma membrane of the photoreceptor, and RPE cells and sub-retinal blebs. In embodiments, the AAV used for sub-retinal administration comprises the capsid protein of AAV214 or AAV214-D5. In embodiments, the sub-retinal administration is for treating ADOA, XLRS, Stargardt disease, Bietti's Crystalline Dystrophy, or BEST vitelliform macular dystrophy. Additional descriptions of sub-retinal administration are disclosed, for example, in Peng et. al., Ophthalmic Res 2017; 58:217-226; and Hartman et al., J Ocul Pharmacol Ther. 2018 Mar. 1; 34 (1-2): 141-153, the contents of each of which are incorporated herein by reference in their entireties for all purposes.

Administration of the AAV viral particle or compositions of this disclosure can be effected in one dose, continuously or intermittently throughout the course of treatment. In embodiments, the AAV viral particle or compositions of this disclosure are parenterally administered by injection, infusion or implantation.

In embodiments, the AAV particles of this disclosure show enhanced tropism for brain and cervical spine. In embodiments, the viral particles of the disclosure can cross the blood-brain-barrier (BBB). In embodiments, the AAV particles of this disclosure show high retinal tropism by para-retinal, sub-retinal and/or intravitreal injections. In embodiments, the AAV particles of this disclosure target multiple eye cell types, such as, for example, cones, rods, and retinal pigment epithelium (RPE). In embodiments, AAV particles of this disclosure escape neutralizing antibodies against natural serotypes, and thus enable potential redosing. In a further aspect, the AAV particles and compositions of the disclosure may be administered in combination with other known treatments for the disorder being treated.

### Kits

The agents, viral vectors, or compositions described herein may, in embodiments, be assembled into pharmaceutical or diagnostic or research kits to facilitate their use in therapeutic, diagnostic or research applications. In embodiments, the kits of the present disclosure include any one of the modified AAV capsid proteins, AAV vectors, AAV viral particles, host cells, isolated tissues, compositions, or pharmaceutical compositions as described herein.

In embodiments, a kit further comprises instructions for use. Specifically, such kits may include one or more agents described herein, along with instructions describing the intended application and the proper use of these agents. As an example, in embodiments, the kit may include instructions for mixing one or more components of the kit and/or isolating and mixing a sample and applying to a subject. In embodiments, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In embodiments, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In embodiments, the preservation solution contains an amount of metalloprotease inhibitors.

In embodiments, the kit contains any one or more of the components described herein in one or more containers. Thus, in embodiments, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to a subject, such as a syringe, topical application devices, or IV needle tubing and bag.

It is to be understood that while the invention has been described in conjunction with the above embodiments, the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

### EXAMPLES

### Example 1: Characterization of Non-Human Primate Para-Retinal and Sub-Retinal Dosing Using Various AAV Viral Vectors

The transduction efficiency of the AAV viral vectors comprising an AAV204, AAV214, AAV214-D5, or AAV8 capsid via multiple ocular administration modes was assessed as described below.

All AAV viral vectors used in this example comprises a recombinant nucleic acid encoding an enhanced green fluorescence protein (“EGFP” or “GFP” hereinafter) reporter transgene that is operably linked to the CBh promoter.

To test transduction efficiency of the AAV viral vectors in vivo, non-human primates (NHP) Macaca fascicularis were dosed by para-retinal, sub-retinal, or intravitreal administration of the indicated AAV viral vectors. The dose/volume for each administration mode were:
Para-retinal dosing—1.0E+11 vg in 100 μL injection volume
Sub-retinal dosing—2.5E+10 vg in 100 μL injection volume
Intravitreal dosing—1.5E+12 vg in 150 μL injection volume
   Para-retinal administration was performed by layering virus on top of the retina between the vitreous and the inner limiting membrane, thus not creating a subretinal detachment. GFP expression was monitored using scanning laser ophthalmoscopy (SLO). SLO images were taken on samples collected 26-27 days post injection. At 28 days post-injection, eyes were collected, processed, and analyzed by immunohistochemistry.

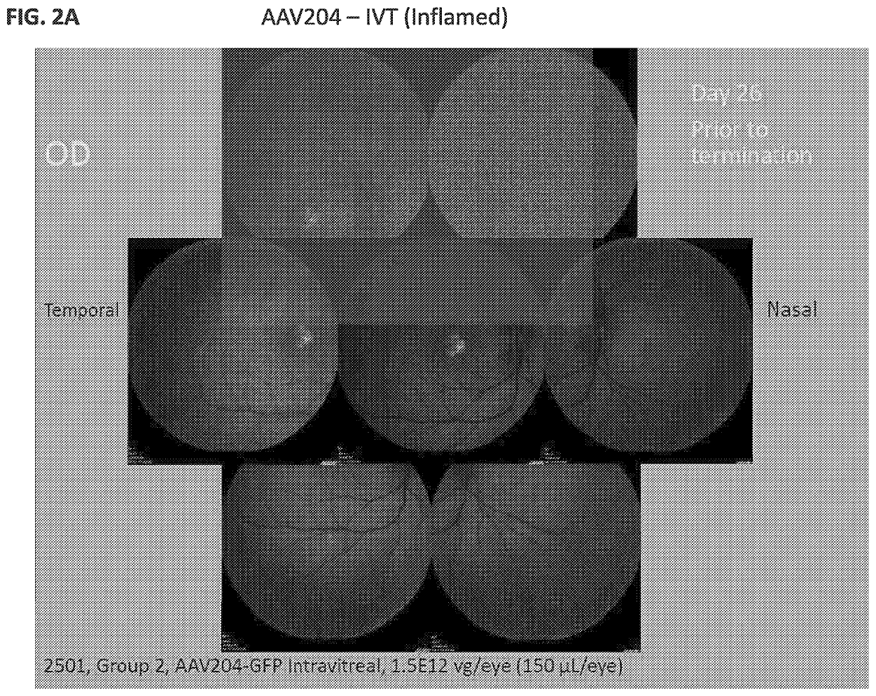

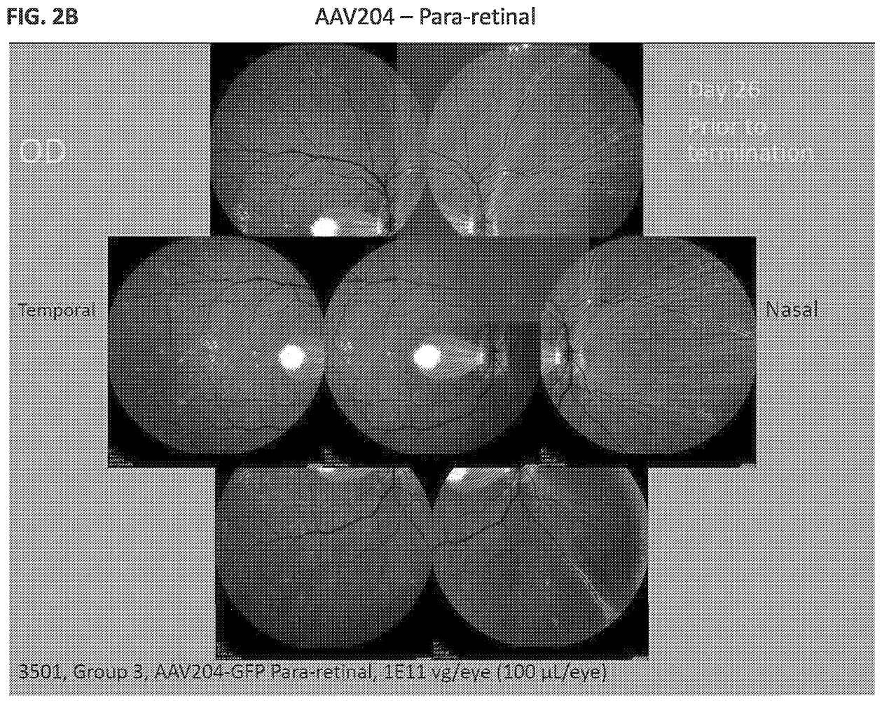

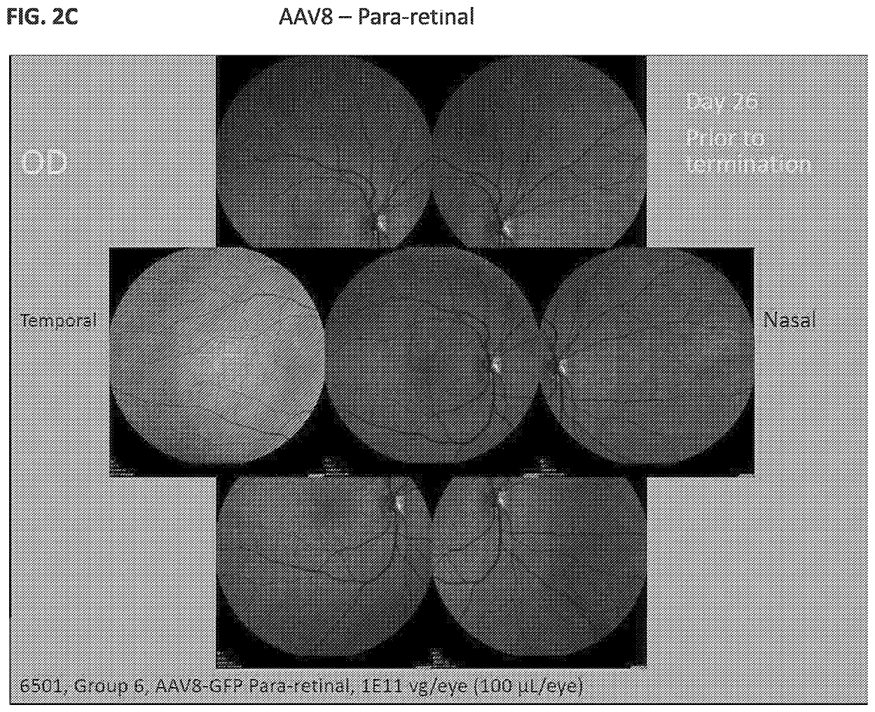

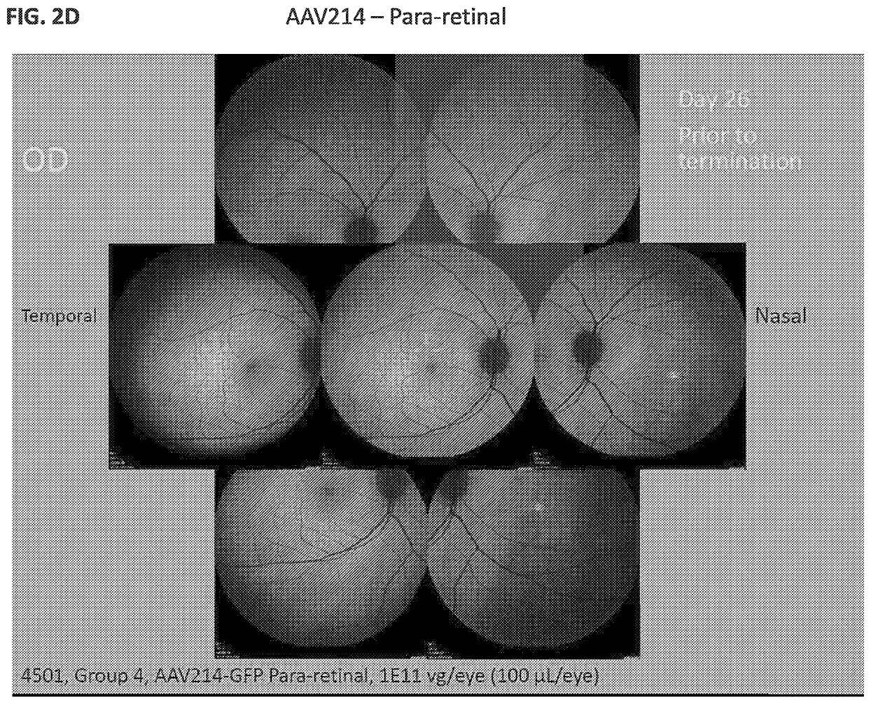

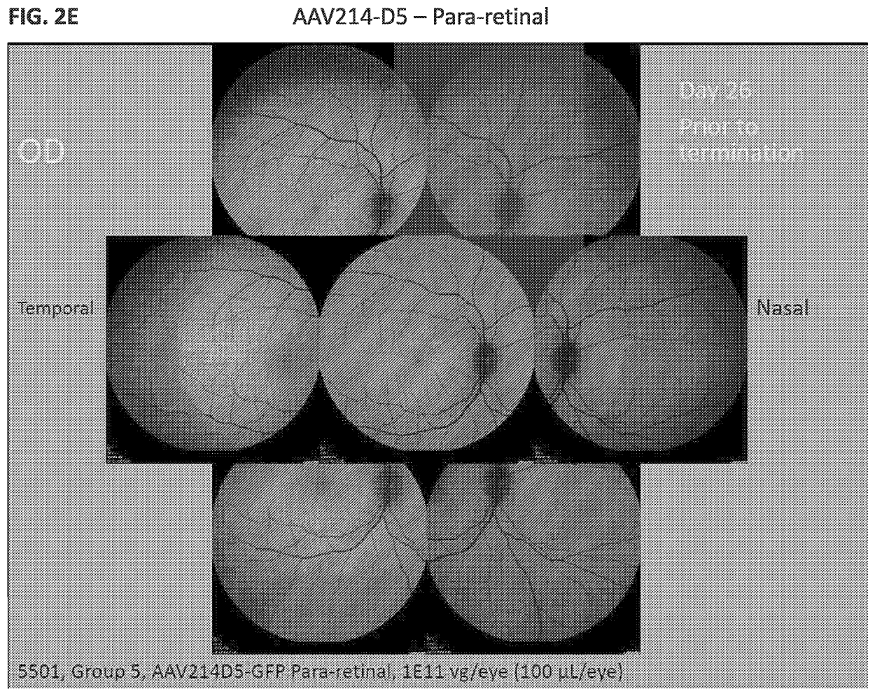

FIGS. 2B-2E shows the SLO results of para-retinal injection of the AAV viral vectors comprising the capsid protein of AAV204 (FIG. 2B), AAV8 (FIG. 2C), AAV214 (FIG. 2D), or AAV214-D5 (FIG. 2E). Among the capsid proteins tested, the AAV viral vector comprising AAV204 capsid demonstrates robust transduction in the macular, papillomacular bundle, and retinal nerve fibers via para-retinal injection, with a much higher transduction efficiency compared to other tested capsids.

In addition, the para-retinal administration of the AAV viral vector comprising AAV204 capsid (FIG. 2B) also shows much higher local transduction efficiency (particularly for optical nerve transduction) compared to the traditional intravitreal administration of the same AAV viral vector (FIG. 2A).

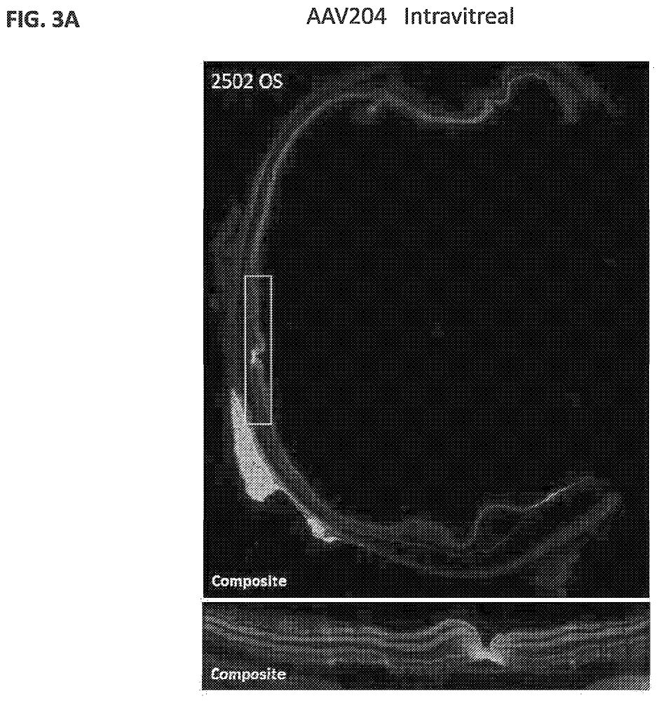

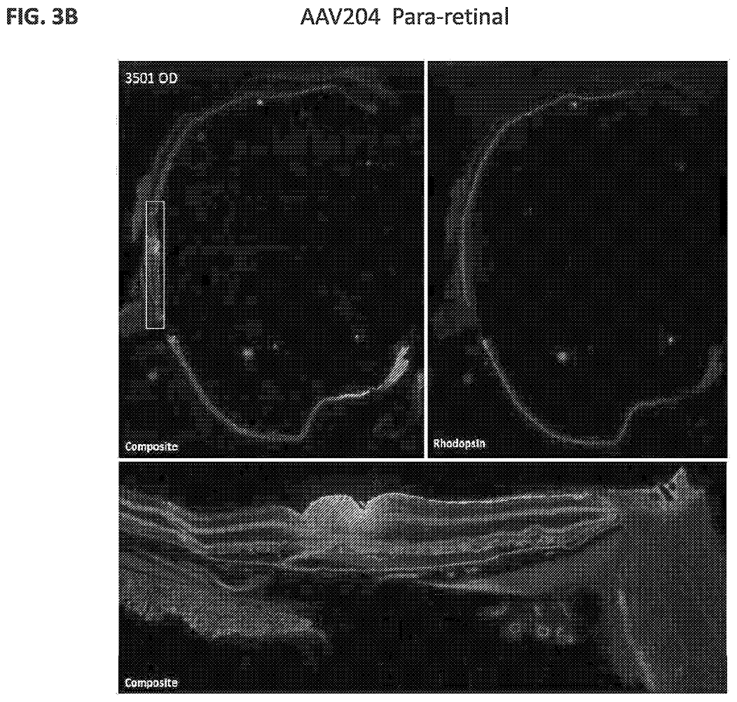

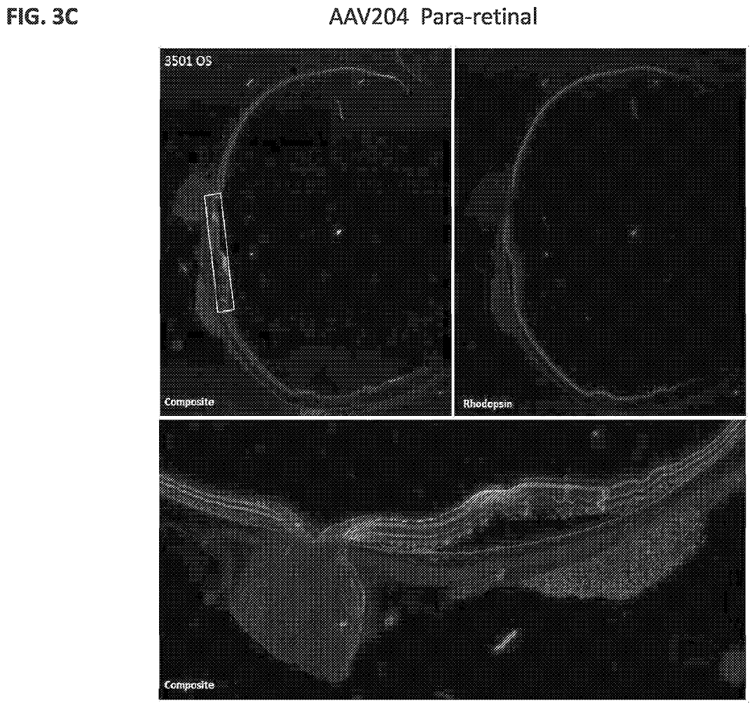

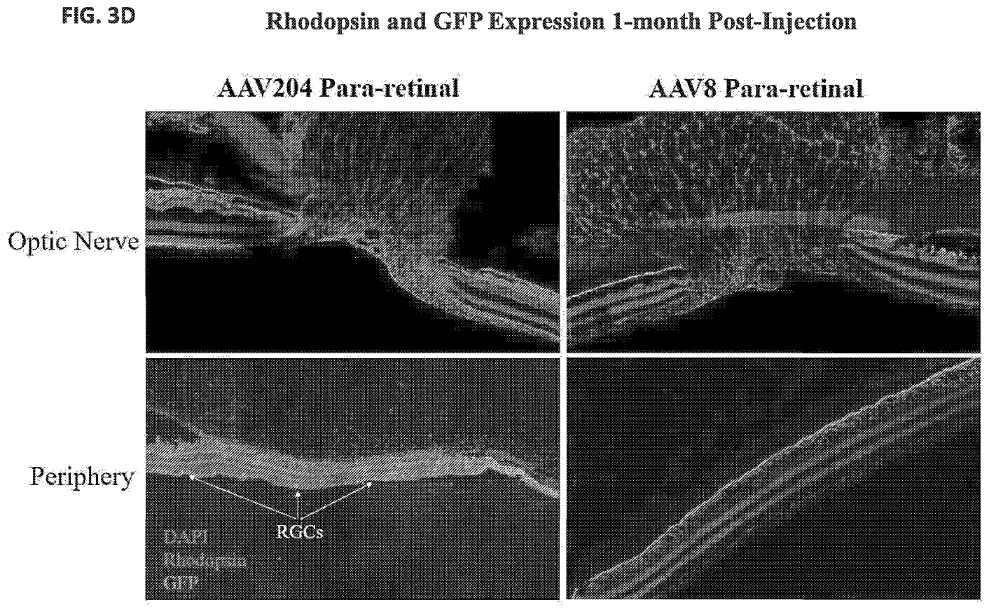

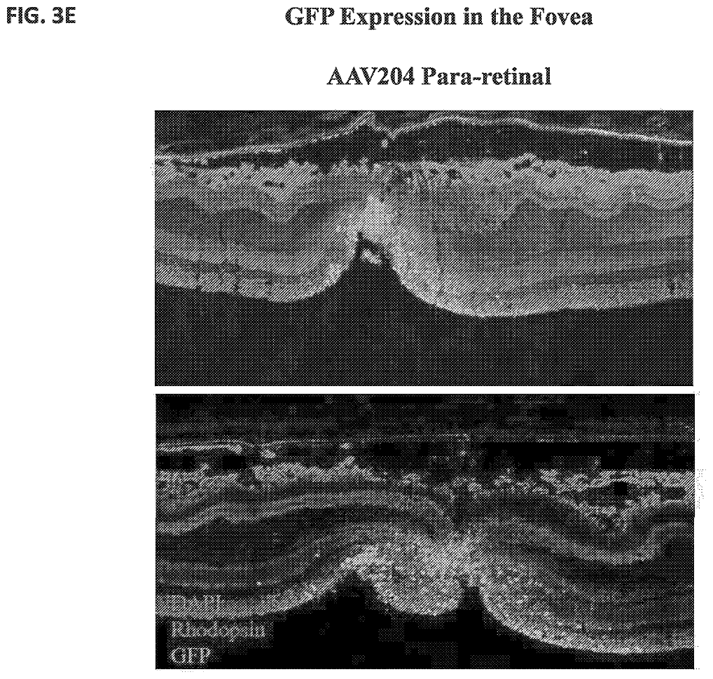

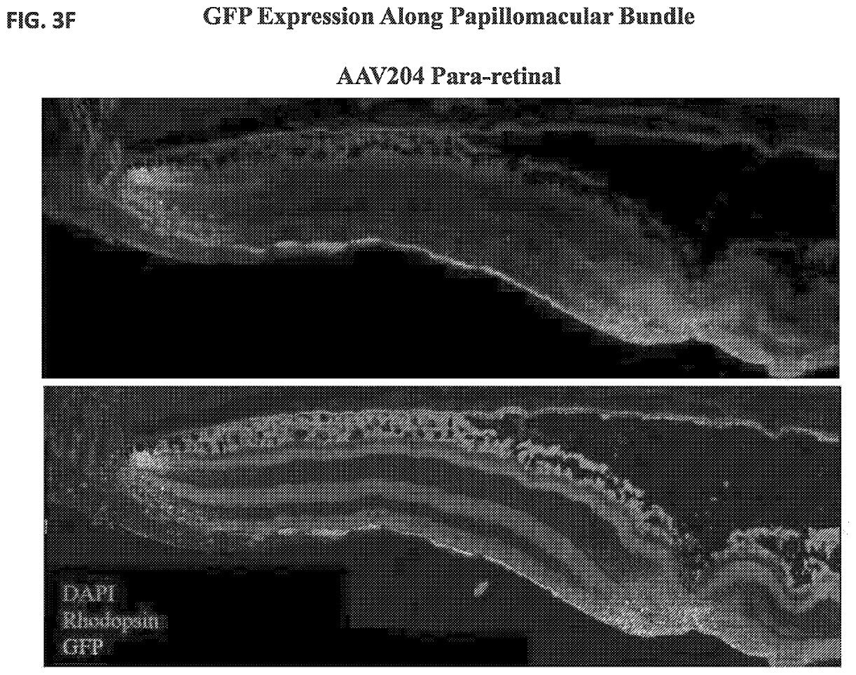

To further compare the transduction efficiency of these two administration routes, the retinas were processed for imaging analysis (FIGS. 3A-3C). Again, retinas receiving para-retinal administration of the AAV viral vector comprising AAV204 capsid (FIGS. 3B-3C) demonstrates much more robust macular and optic nerve transduction than the retinas receiving intravitreal administration of the same AAV viral vector (FIG. 3A).

Further immunohistochemistry analysis of rhodopsin and GFP 1-month post para-retinal injection of AAV204 viral vectors (FIG. 3D) showed high GFP expression in retinal ganglion cells (RGCs) across the entire retina and nerve fibers with high GFP expression were observed along the retina and entering the optic nerve. In comparison, para-retinal injection of AAV8 viral vectors (FIG. 3D) showed much lower GFP expression in RGCs. As shown in FIGS. 3E-3F, para-retinal administration of AAV204 viral vectors also resulted in robust GFP expression in NHP fovea and along the the papillomacular bundle between the macula and optic nerve. These results were consistent with the SLO analysis. Thus, para-retinal administration of AAV204 viral vector results in efficient transduction of target cells in the macula and foveal pit as well as retinal ganglion cells and the associated retinal nerve fibers extending to the optic nerve, at a dose that is at least 10-fold lower compared to intravitreal AAV injections commonly used in the field.

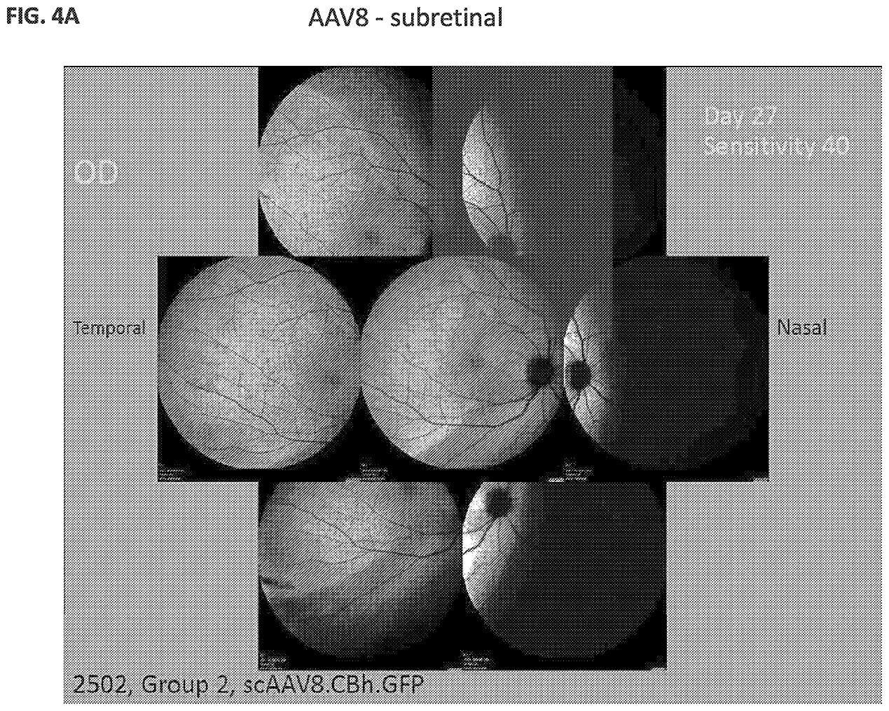

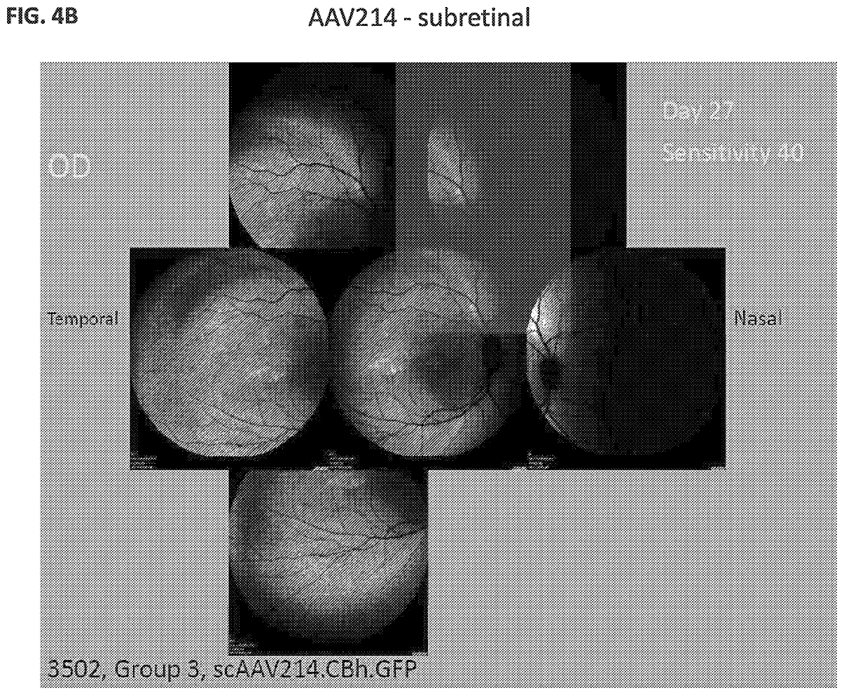

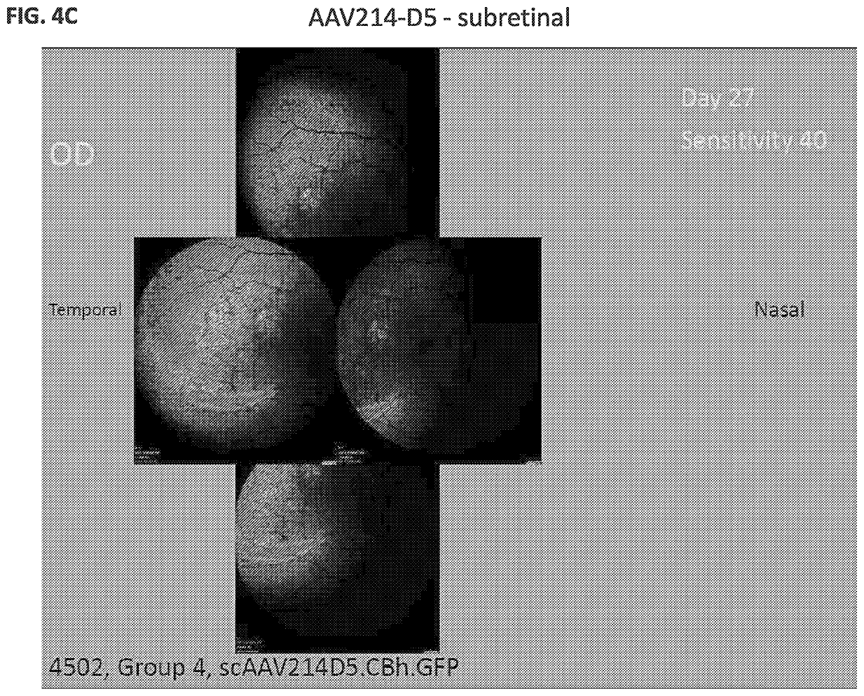

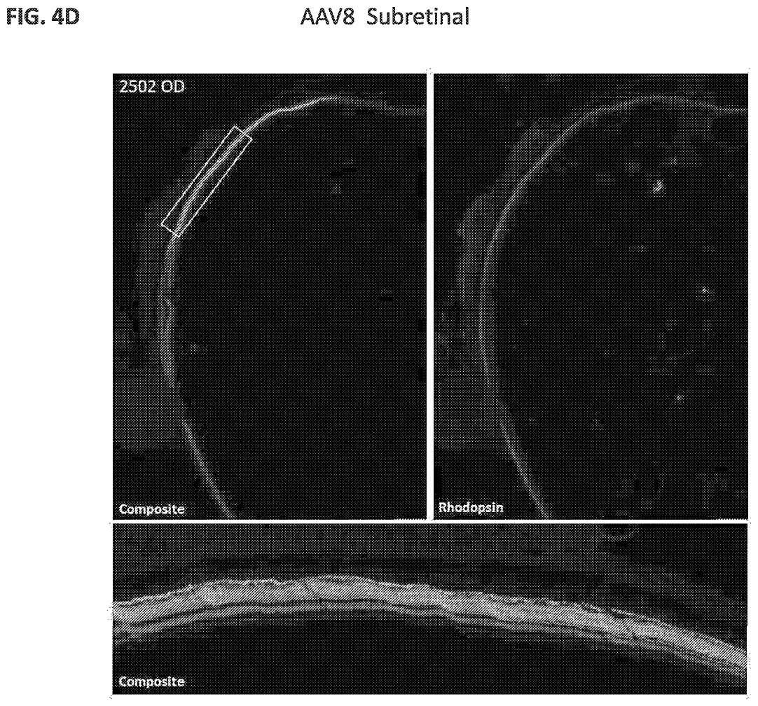

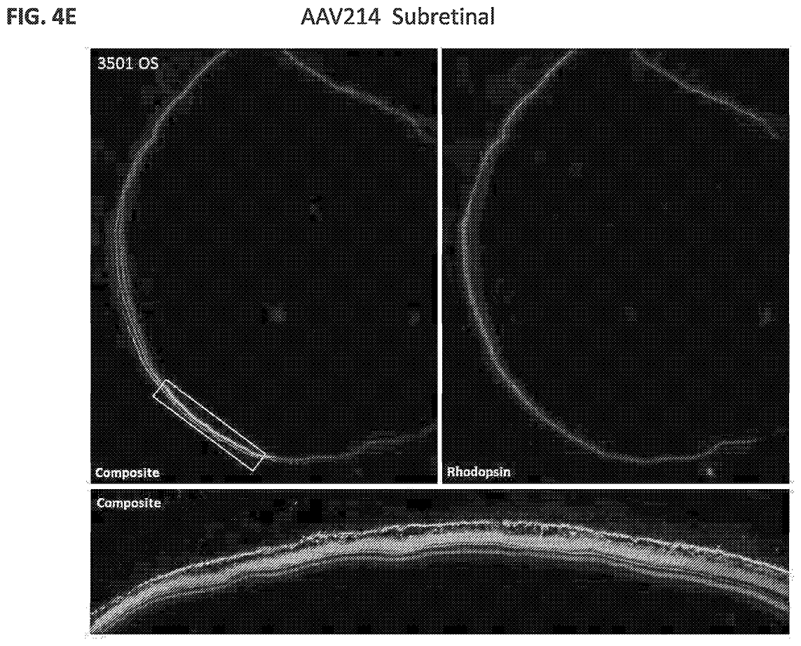

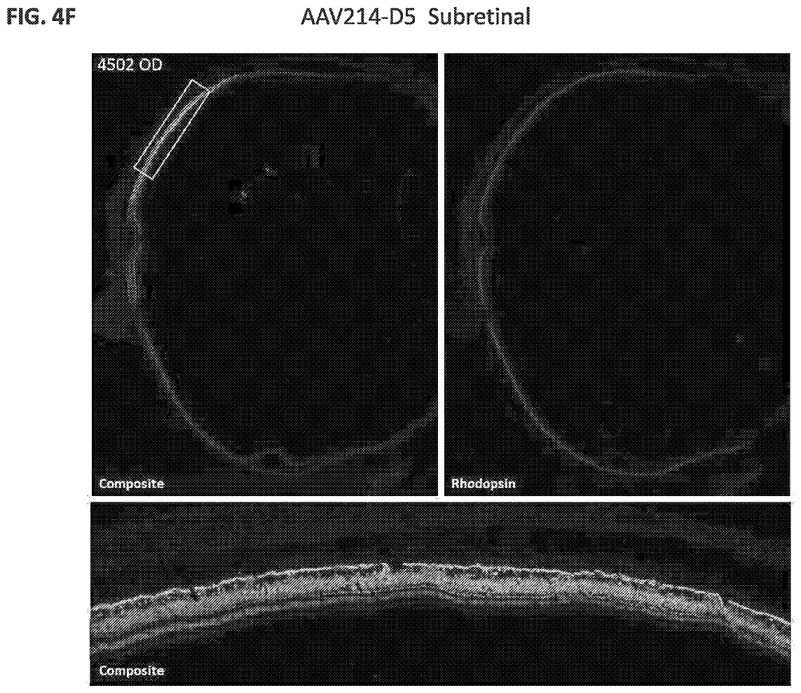

The transduction efficiency of the AAV viral vectors comprising the capsid protein of AAV8 (FIGS. 4A, 4D), AAV214 (FIGS. 4B, 4E), and AAV214-D5 (FIG. 4C, 4F) was also assessed. The results show that these 3 capsids display similar transduction efficiency when administered sub-retinally.

Conclusions: These results show that para-retinal injection of AAV vectors comprising AAV204 capsid can efficiently deliver the payload to the macula or optic nerve/retinal ganglion cell layer.

### Example 2: In Vitro Characterization of AAV Vector Genomes Comprising Human Opa1 Expression Cassette

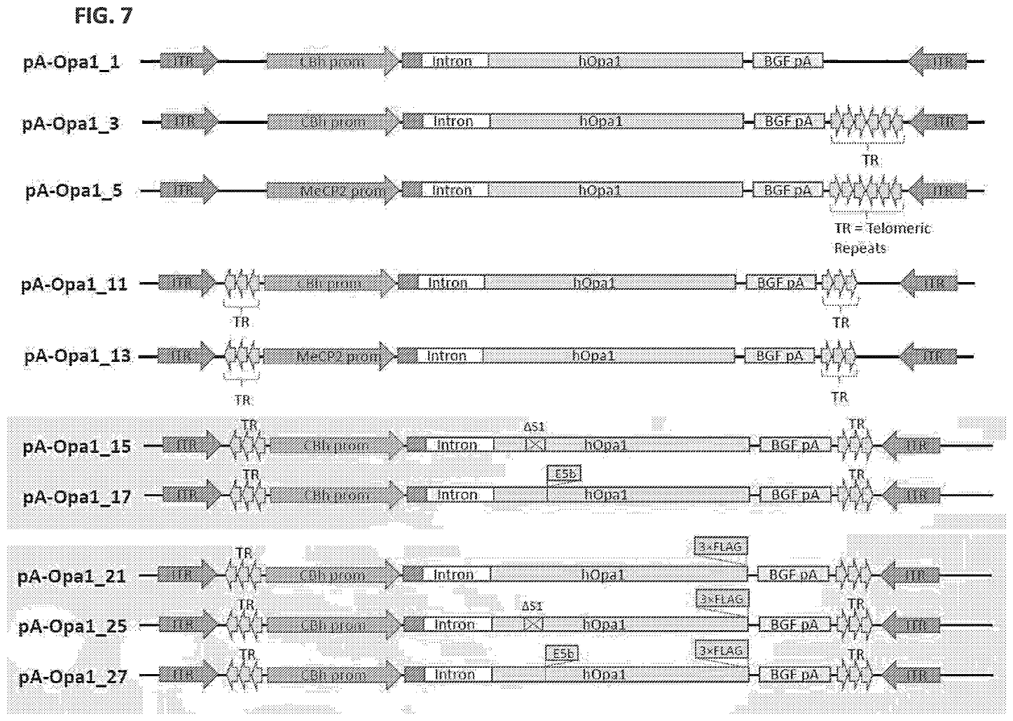

Multiple AAV vector genomes expression vectors were constructed for expression of human Opa1 (hOpa1) according to FIG. 7, including:
pA-Opa1_1 (comprising SEQ ID NO: 230; generated from a DNA template vector comprising SEQ ID NO: 186);
pA-Opa1_3 (comprising SEQ ID NO: 231; generated from a DNA template vector comprising SEQ ID NO: 187);
pA-Opa1_5 (comprising SEQ ID NO: 232; generated from a DNA template vector comprising SEQ ID NO: 188);
pA-Opa1_11 (comprising SEQ ID NO: 233; generated from a DNA template vector comprising SEQ ID NO: 189);
pA-Opa1_13 (comprising SEQ ID NO: 234; generated from a DNA template vector comprising SEQ ID NO: 190);
pA-Opa1_15 (comprising SEQ ID NO: 235; generated from a DNA template vector comprising SEQ ID NO: 191);
pA-Opa1_17 (comprising SEQ ID NO: 236; generated from a DNA template vector comprising SEQ ID NO: 192);
pA-Opa1_21 (comprising SEQ ID NO: 237; generated from a DNA template vector comprising SEQ ID NO: 193);
pA-Opa1_25 (comprising SEQ ID NO: 238; generated from a DNA template vector comprising SEQ ID NO: 194); and
pA-Opa1_27 (comprising SEQ ID NO: 239; generated from a DNA template vector comprising SEQ ID NO: 195).

Each vector genome construct comprises, from 5′ to 3′, a promoter (CBh promoter (SEQ ID NO: 154) or MeCP2 promoter (SEQ ID NO: 156)), an intron (SEQ ID NO: 200), an hOpa1 open reading frame, and BGH polyA signal (SEQ ID NO: 201 or 225), which are flanked by 5′ and 3′ ITRs. In addition, most constructs contained telomeric repeats (TR) downstream of the BGH polyA signal and optionally upstream of the promoter. Specifically, pA-Opa1_1 contains no TR; both pA-Opa1-3 and pA-Opa1-5 contain a first TR (SEQ ID NO: 202) and a second TR (SEQ ID NO: 203) downstream of the BGH poly A signal; and the other AAV vector genomes contain both a first TR (SEQ ID NO: 202) downstream of the BGH polyA signal and a second TR (SEQ ID NO: 203) upstream of the promoter. The Opa1 transgene is operably linked to CBh promoter in most AAV vector genomes, except that in pA-Opa1_5 and pA-Opa1_13 the Opa1 transgene is operably linked to MeCP2 promoter. In addition, the transgene in pA-Opa1_15 and pA-Opa1_25 is human Opa1 ΔS1 isoform, and the transgene in pA-Opa1_17 and pA-Opa1_27 is human Opa1 E5b isoform. The pA-Opa1_2× constructs further comprise a 3×FLAG tag fused to the 3′ end of the human Opa1 open reading frame.

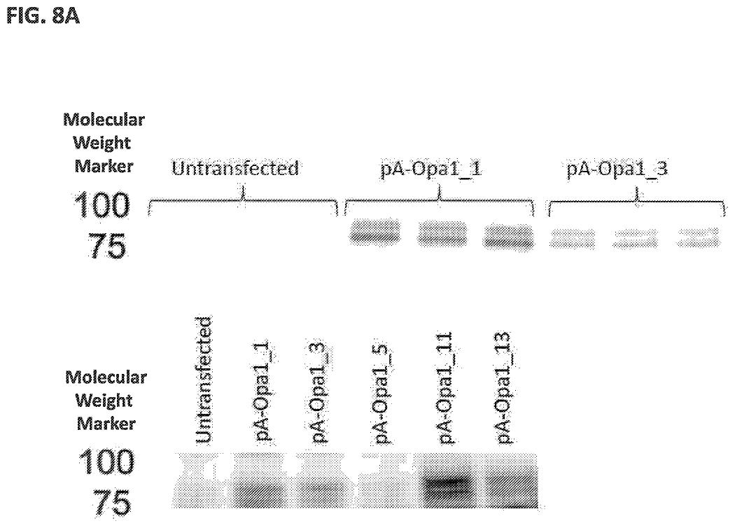

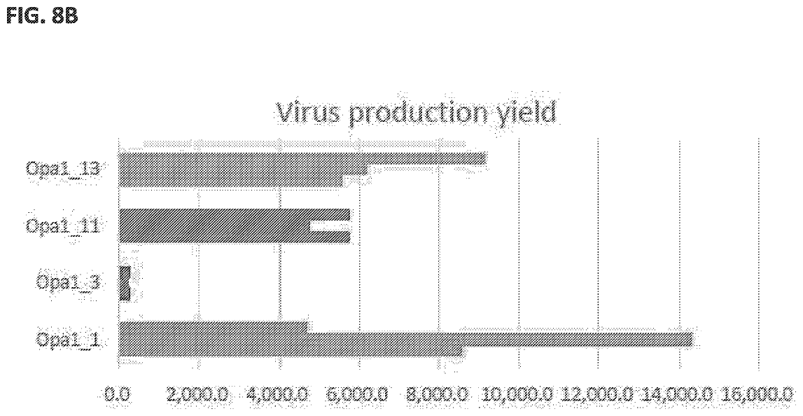

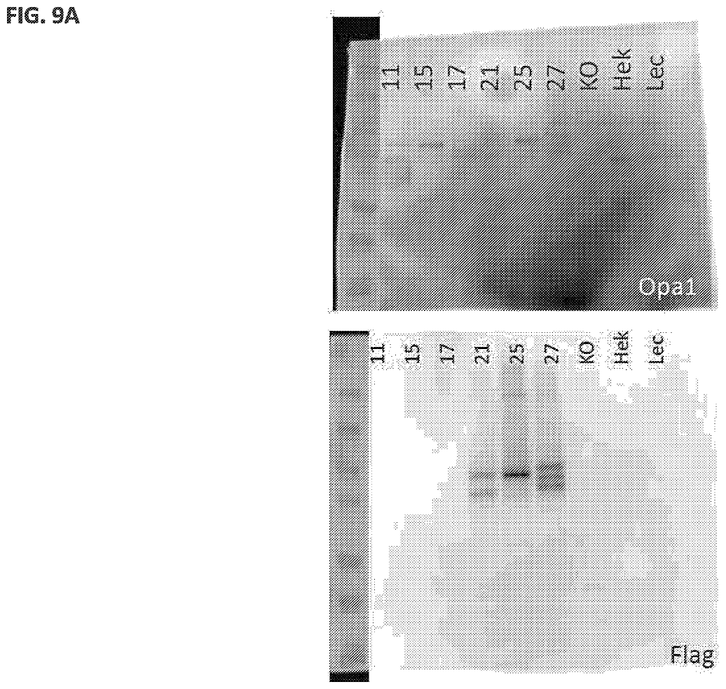

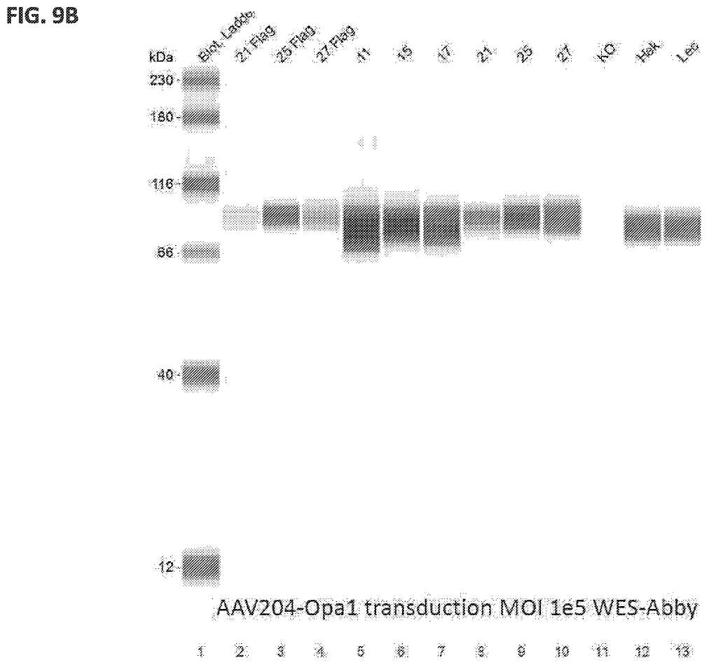

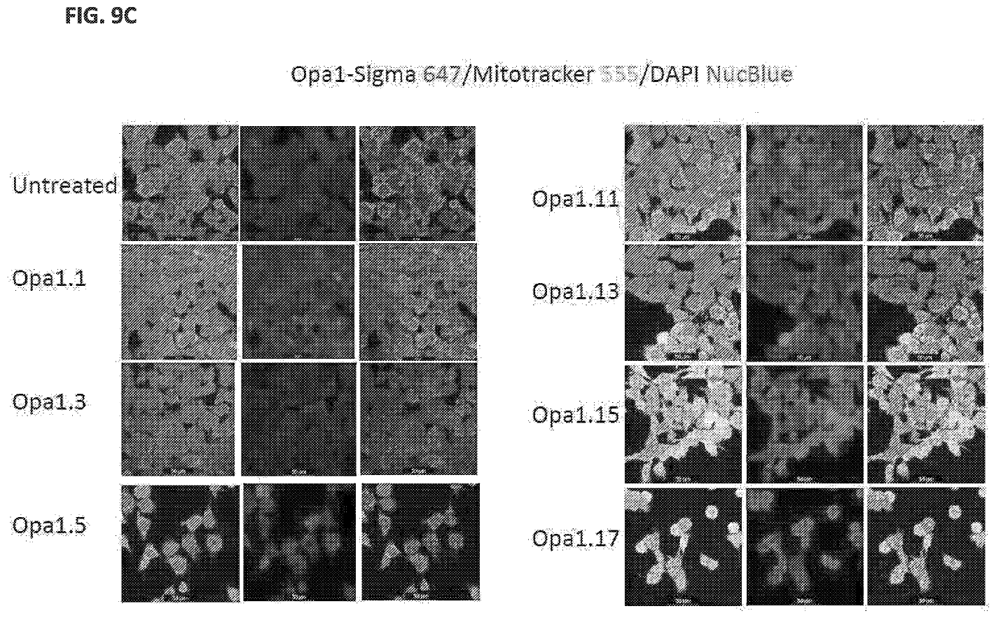

After preparation of plasmid DNA of each AAV vector genome, 293 cells were transfected with 1 μg of plasmid DNA and incubated at 37° C., 5% CO₂ for 48 hours prior to collection of protein lysates. Opa1 expression was examined using Western analysis (FIG. 8A). In addition, the AAV capsid packaging capability of these AAV vector genomes were analyzed by measuring virus production yield (FIG. 8B; the X-axis shows the relative units corresponding to DNase resistant viral genome number in a given volume of virus production medium). The ability of AAV viral vectors to infect cells and express Opa1 protein was assessed for the indicated vector genomes. Opa1 expression by each vector genome was detected by an anti-Opa1 antibody (FIG. 9A, upper panel and FIG. 9B, lanes 5-13) or by anti-FLAG antibody (FIG. 9A, lower panel and FIG. 9B, lanes 2-4). In addition, Opa1 expression was also analyzed by protein staining in transfected Opa1 (−/−) cell line (FIG. 9C).

The results showed that pA-Opa1_3 produced about 4-fold lower Opa1 expression compared to pA-Opa1_1 in 293 cells, and the expression from pA-Opa1_5 was even lower than pA-Opa1_3. Neither pA-Opa1_3 nor pA-Opa1_5 was capable of efficiently packaging into an AAV capsid. On the other hand, pA-Opa1_11 yielded better Opa1 expression than pA-Opa1_1, and pA-Opa1_13 also showed better Opa1 expression compared to pA-Opa1_5. In addition, both pA-Opa1_11 and pA-Opa1_13 were capable of efficiently packaging into an AAV capsid. Thus, introducing the telomeric repeats flanking both 5′ and 3′ side of the Opa1 expression cassette improves Opa1 expression as well as packaging of the AAV vector genome.

### Example 3: In Vivo Study of Dominant Optic Atrophy Treatment Using AAV Viral Vectors Encoding OPA1

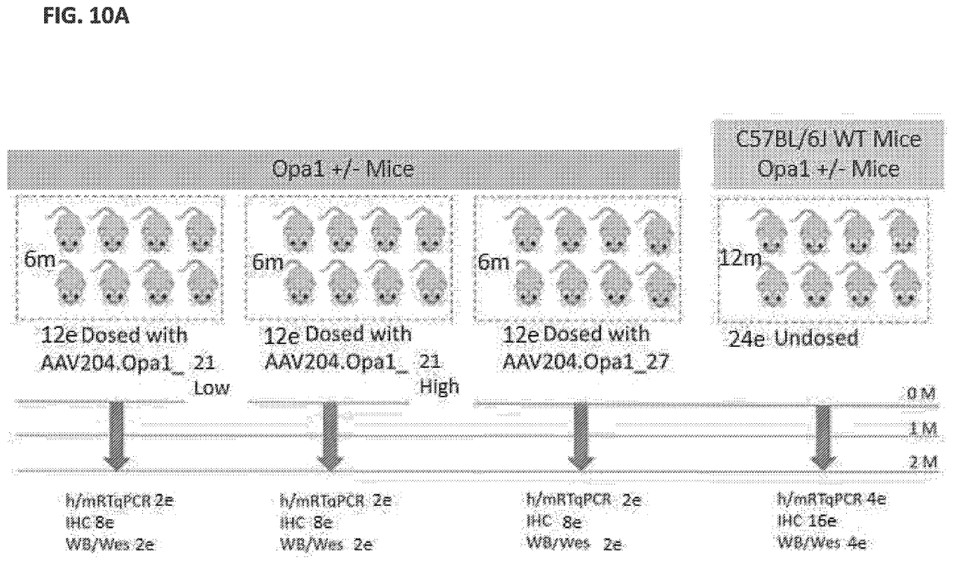

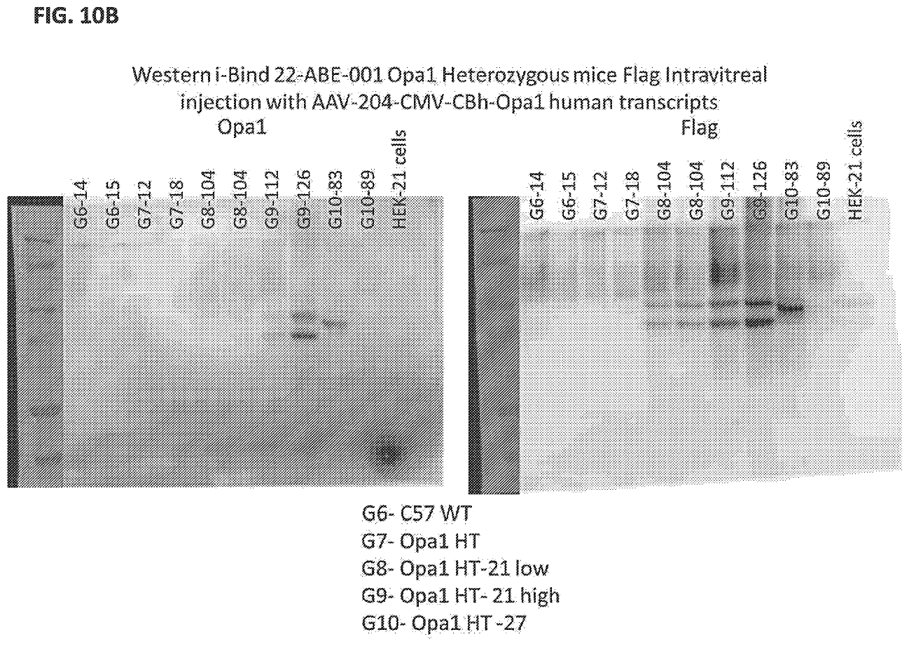

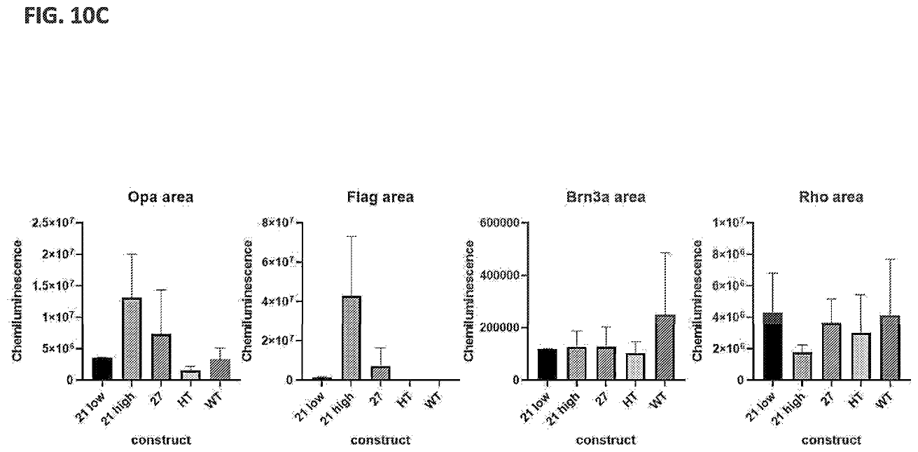

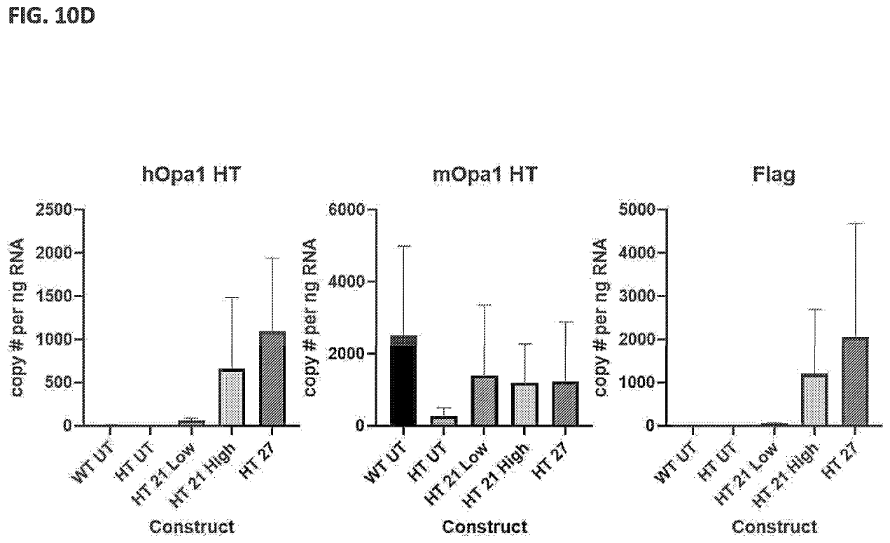

To investigate Opa1 expression in vivo, animal studies were conducted according to FIG. 10A. Briefly, Opa1 −/+ mice in three treatment groups were injected at one month of age with AAV204 viral vector containing pA-Opa1_21 (high or low dose), or pA-Opa1_27, according to Table 7 below via intravitreal injection into the retinal vitreal space with a 33-34G needle and syringe. Left eyes were penetrated on the temporal side and right eyes on the nasal side of the pupil. Optical Coherence Tomography (OCT) was performed post injection to assess for any post-operative damage and either buprenorphine (0.01-0.05 mg/ml) or meloxicam (5 mg/ml) was administered to assist in recovery from anaesthesia. Post-procedure either ofloxacin or neyomycin polymyxin B gramicidin was administered to prevent infection. Mice were also administered atipamezole (0.1-1.0 mg/kg) to reverse adverse effects from metabolizing xylazine. The mice were then allowed to recover on heating pads until full consciousness. Mice were monitored post-injection for morbidity and mortality daily for two months. Six mice were used for each of three treatment groups and two control untreated groups (30 mice total). They were sacrificed at three months of age and mice were euthanized via CO2 exposure and eyes were removed first with blunt scissors to cut away the eyelids followed by curved tweezers and 1 mm surgical scissors to separate the optic nerve. The cornea was cut and the lens removed to create the “eye cups” samples for subsequent analysis.

TABLE 7
Mice Cohorts for Intravitreal Injection

| | | | | Dose | Necropsy | |
|---|---|---|---|---|---|---|
| | # of | Dosed | | (vg of | (28 days PI) | |
| | Mice | eyes | Test Article | virus) 1 ul | Frozen | IHC |
| Cohort 1 | 8 | 0 of 16 | None | 0 | 12 eyes | 4 eyes |
| Cohort 2 | 8 | 16 of 16 | AAV204.Opa1_21 | 1.0E+9 | 12 eyes | 4 eyes |
| | | | Low dose | | | |
| Cohort 3 | 8 | 16 of 16 | AAV204.Opa1_21 | 3.0E+9 | 12 eyes | 4 eyes |
| | | | High dose | | | |
| Cohort 4 | 8 | 16 of 16 | AAV204.Opa1_27 | 3.0E+9 | 12 eyes | 4 eyes |

Upon assaying for Opa1-Flag expression, expression of the Opa1-21 protein at the correct size, in the correct isoform of two bands, was observed in all treated animals (FIG. 10B). The isoform pattern is identical to the endogenous isoform in the untreated and treated wild-type eyes (although the wildtype bands were only visible upon overexposure). The Opa1-27 protein was expressed in the eye samples but not in the predicted isoform size as in cell culture. The expression of Opa1-27 protein seemed to be limited to the un-cleaved long/S1 isoform(s), without the short isoform(s) that may regulate mitochondrial fusion (as described in Wang et al., Mol Biol Cell. 2021 Jan. 15; 32 (2): 157-168). The amount of Brn3a and Rhodopsin (Rho) in the samples were also analyzed using an equivalent protein load (FIG. 10C). Brn3a, the marker for retinal ganglion cells, was more consistent between samples.

These results show that, compared to the wildtype (WT) animal, all three treatment groups showed a significant increase in the expression of heterologous Opa1, with the construct pA-Opa1-21 high-dose group achieving the most significant Opa1 expression. The Opa1 expression data correlated with the levels of FLAG-tag expression form all three groups with a clear absence for FLAG expression from the untreated samples.

In addition to Western analysis, RT-PCR was used to analyze the expression level of the RNA transcript. As shown in FIG. 10D, significant expression of RNA transcripts of both pA-Opa1_21 (isoform 1) high and low dose, as well as pA-Opa1_27 (isoform 7), was observed in all treated HT animals, whereas, as expected, transcripts encoding human Opa1 or FLAG-tag were not detected in untreated animals. Interestingly, even though pA-Opa1_27 appeared to be expressing at higher levels on the mRNA level, the protein level was much lower than the high dose of pA-Opa1_21.

On the other hand, construct pA-Opa1_25 yielded no detectable expression of Opa1 at either mRNA or protein level (data not shown).

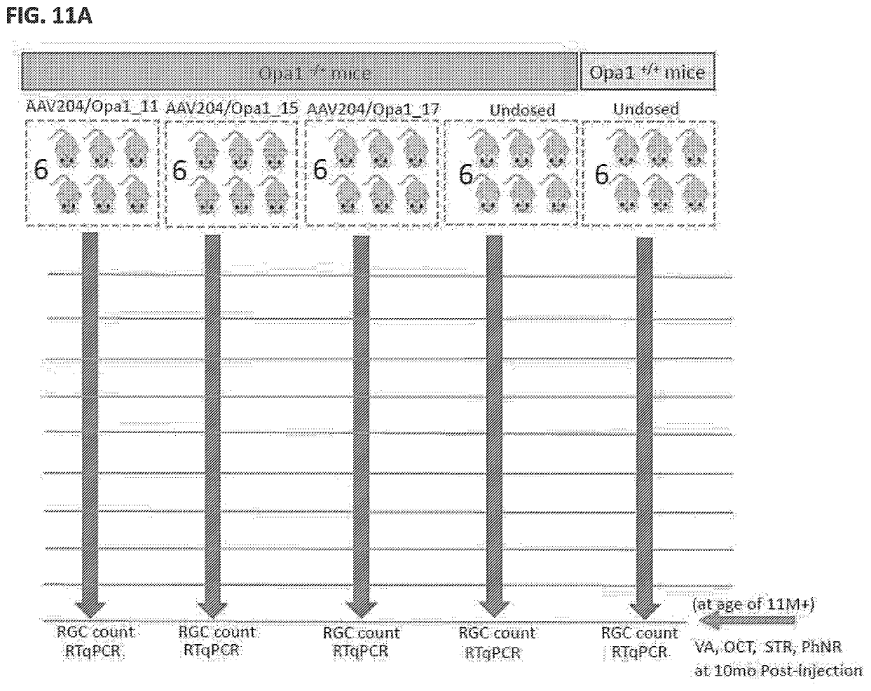

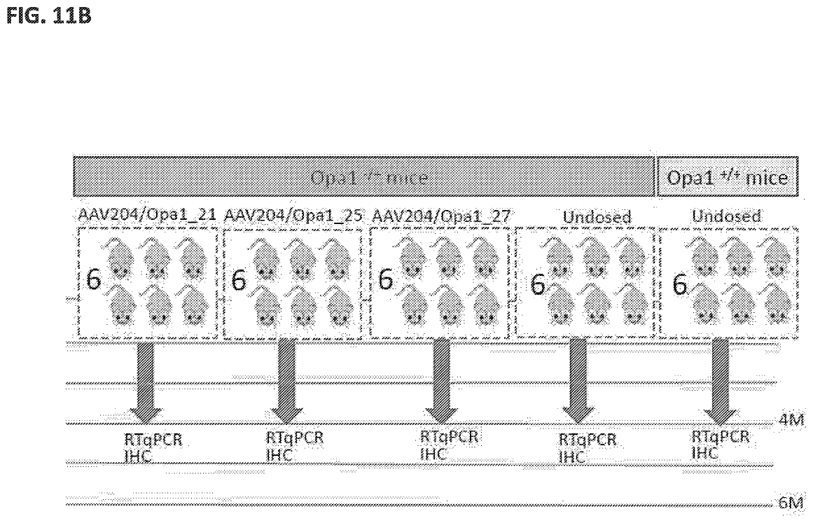

In another proposed study (FIGS. 11A and 11B), Opa1 −/+ mice in each treatment group are dosed with AAV204 viral vector comprising the indicated vector genome. The two control groups are undosed Opa1 −/+ mice and undosed Opa1 +/+ mice, respectively. For the treatment groups delivered with Opa1 transgene without 3×FLAG tag, Opa1 expression level and treatment efficacy are evaluated by RT-qPCR and counting the retinal ganglion cells (RGCs) (FIG. 11A). Visual acuity (VA), Optical Coherence Tomography (OCT), Scotopic Threshold Response (STR), and photopic negative response (PhNR) are measured at 10-month post-injection. In addition, and for the treatment groups delivered with Opa1 transgene containing 3×FLAG tag, Opa1 expression is evaluated using RT-qPCR and IHC at 4-month post injection (FIG. 11B).

### Example 4: In Vitro Characterization of AAV Vector Genomes Comprising Human RS1 Expression Cassette

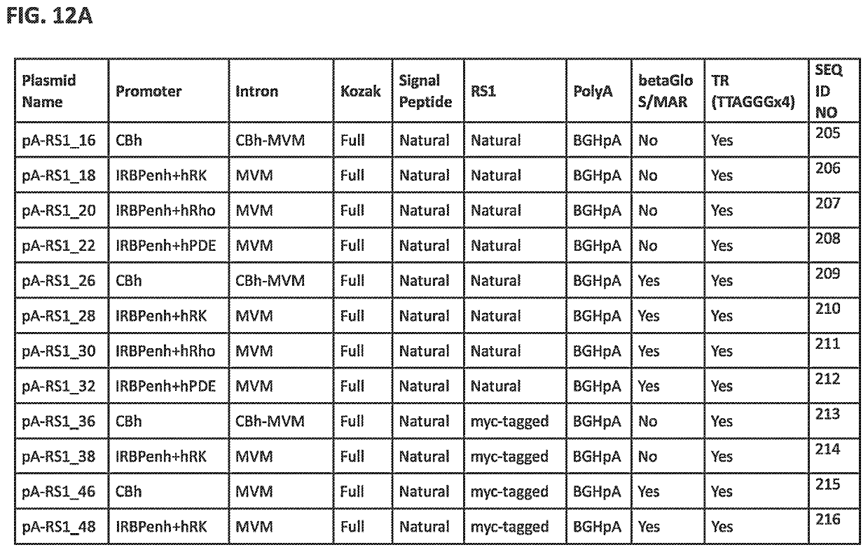

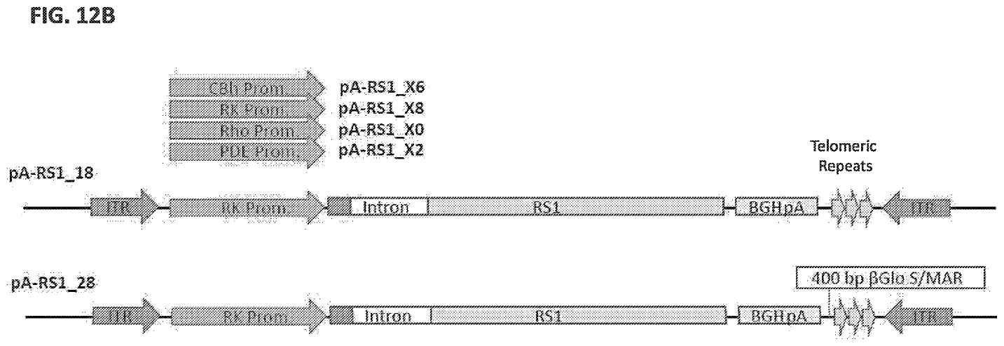

Multiple AAV vector genomes were constructed for expression of RS1 protein according to FIG. 12A, with overall design illustrated in FIG. 12B. Each construct comprises, from 5′ to 3′, a promoter, an intron of either CBh-MVM (SEQ ID NO: 200) or MVM (SEQ ID NO: 222), a RS1 open reading frame, a BGH polyA site (SEQ ID NO: 201 or 225), and a telomeric repeat (SEQ ID NO: 203), which are flanked by 5′ and 3′ ITRs. For the promoter, each construct ending in “6” comprises a CBh promoter (SEQ ID NO: 154), each construct ending in “8” comprises a RK promoter (SEQ ID NO: 196), each construct ending in “0” comprises a Rho promoter (SEQ ID NO: 197), and each construct ending in “2” comprises a PDE promoter (SEQ ID NO: 198). Some constructs also contains a betaGlo_s/MAR sequence (SEQ ID NO: 221) between the BGH polyA site and the telomeric repeat.

The AAV vector genomes used in this study include:
pA-RS1_8 (comprising SEQ ID NO: 240; generated from a DNA template vector comprising SEQ ID NO: 204);
pA-RS1_16 (comprising SEQ ID NO: 241; generated from a DNA template vector comprising SEQ ID NO: 205);
pA-RS1_18 (comprising SEQ ID NO: 242; generated from a DNA template vector comprising SEQ ID NO: 206);
pA-RS1_20 (comprising SEQ ID NO: 243; generated from a DNA template vector comprising SEQ ID NO: 207);
pA-RS1_22 (comprising SEQ ID NO: 244; generated from a DNA template vector comprising SEQ ID NO: 208);
pA-RS1_26 (comprising SEQ ID NO: 245; generated from a DNA template vector comprising SEQ ID NO: 209);
pA-RS1_28 (comprising SEQ ID NO: 246; generated from a DNA template vector comprising SEQ ID NO: 210);
pA-RS1_30 (comprising SEQ ID NO: 247; generated from a DNA template vector comprising SEQ ID NO: 211);
pA-RS1_32 (comprising SEQ ID NO: 248; generated from a DNA template vector comprising SEQ ID NO: 212);
pA-RS1_36 (comprising SEQ ID NO: 249; generated from a DNA template vector comprising SEQ ID NO: 213);
pA-RS1_38 (comprising SEQ ID NO: 250; generated from a DNA template vector comprising SEQ ID NO: 214);
pA-RS1_46 (comprising SEQ ID NO: 251; generated from a DNA template vector comprising SEQ ID NO: 215);
pA-RS1_48 (comprising SEQ ID NO: 252; generated from a DNA template vector comprising SEQ ID NO: 216); and
pA-RS1_58 (comprising SEQ ID NO: 224; generated from a DNA template vector comprising SEQ ID NO: 223).

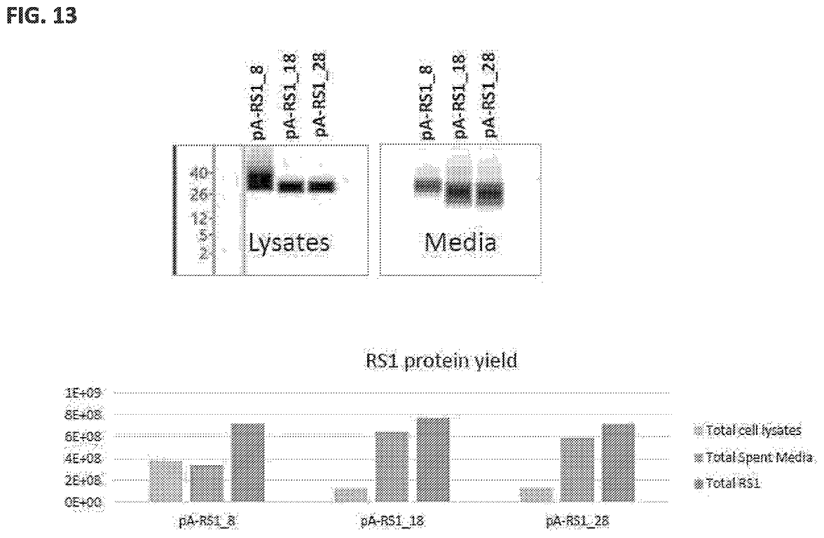

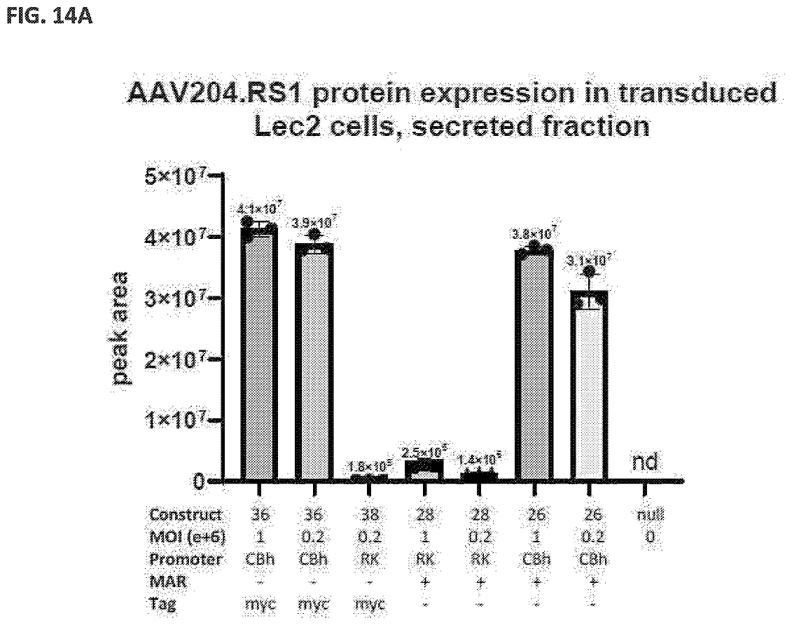

The RS1 protein expression levels of three AAV vector genomes (pA-RS1_8, pA-RS1_18, and pA-RS1_28) were evaluated using Western analysis (FIG. 13). Both pA-RS1_18 and pA-RS1_28 yielded higher fraction of secreted RS1 protein compared to pA-RS1_8, although the total protein yields were comparable among these constructs.

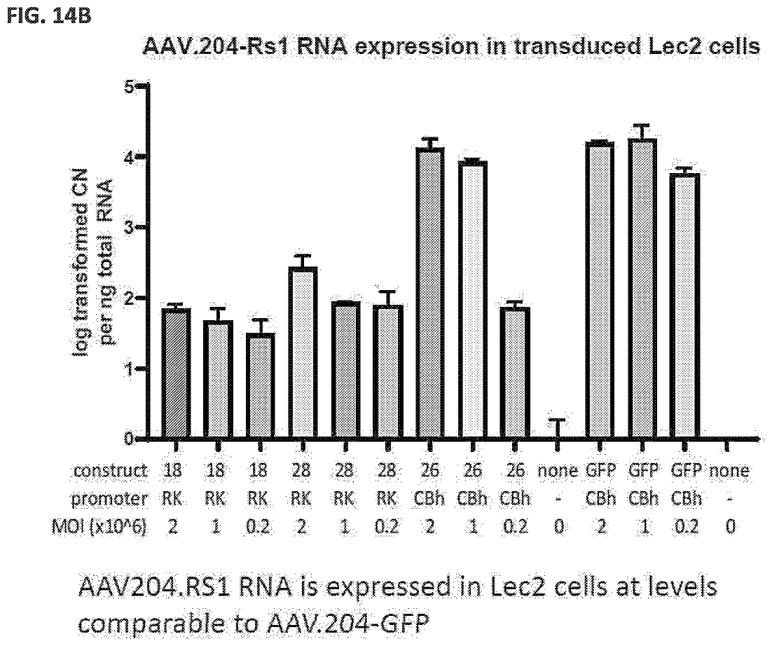

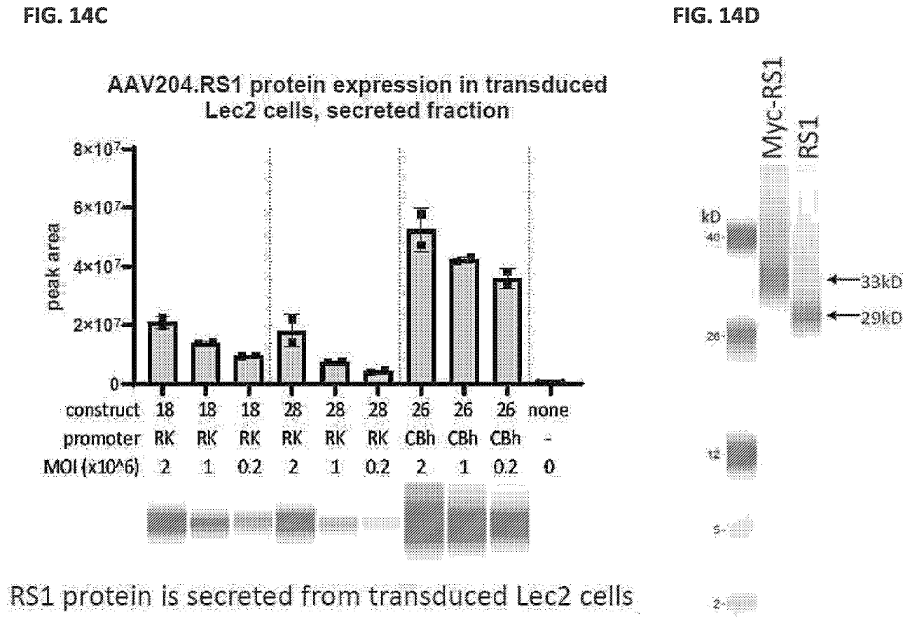

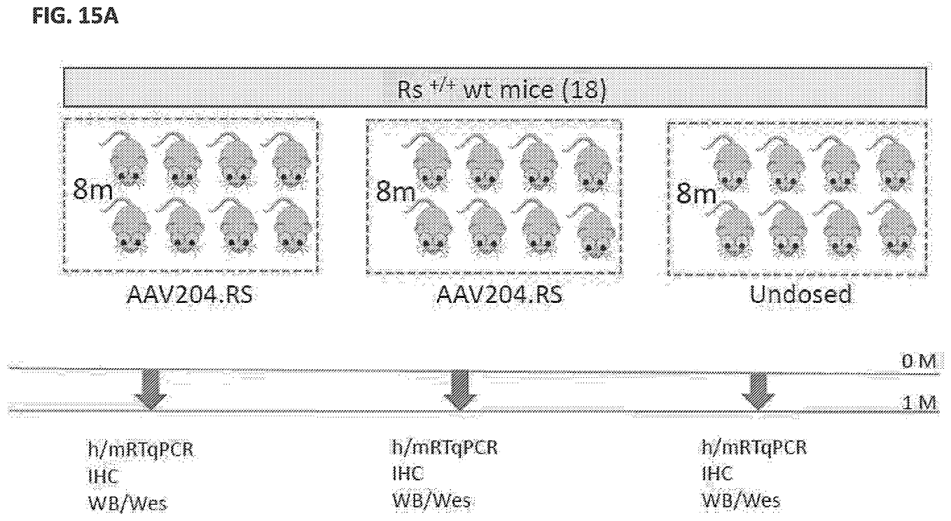

Potency of AAV204 viral vector comprising indicated RS1-expressing vector genome was also evaluated in Lec2 cell culture. As shown in FIGS. 14A-14C, myc-tagged RS1 was expressed at levels similar to untagged RS1 in transduced Lec2 cells and was appropriately secreted. And the CBh promoter enabled higher expression of RS1 compared to the RK promoter. The secreted myc-RS1 is about 4 kD larger than native RS1 (FIG. 14D).

### Example 5: In Vivo Study of X-Linked Retinoschisis Treatment Using AAV Viral Vectors Encoding RS1

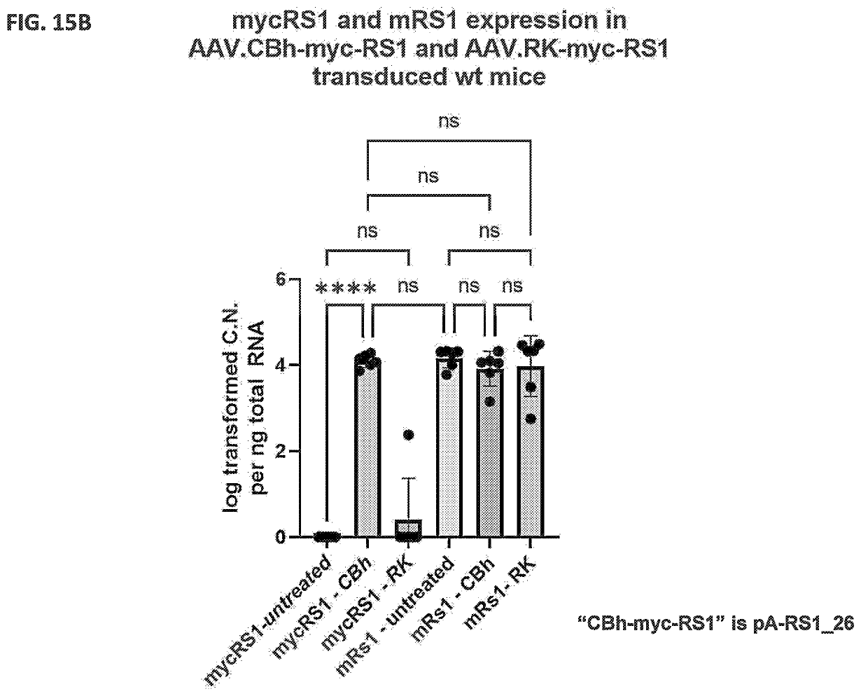

In one study, wildtype mice were administered with AAV204 viral vector comprising the RS1 expression cassette via intravitreal injection. The expression of RS1 protein in each animal group was evaluated by RT-qPCR (mRNA copy number/ng of total RNA.) and Western analysis, and the protein distribution by IHC (FIG. 15A), at 1-month time point post injection.

As shown in FIG. 15B, expression of mouse RS1 (mRS1) protein was detected in each tested mouse, and the mRS1 expression mean values were not statistically different among the test groups. Mice administered with AAV204.pA-RS1_26 exhibits robust expression of myc-tagged human RS1 (hRS1) at a level comparable to natural mRS1 expression. On the other hand, there was little detectable expression of hRS1 from the RK promoter at this time point in mice (with one exception).

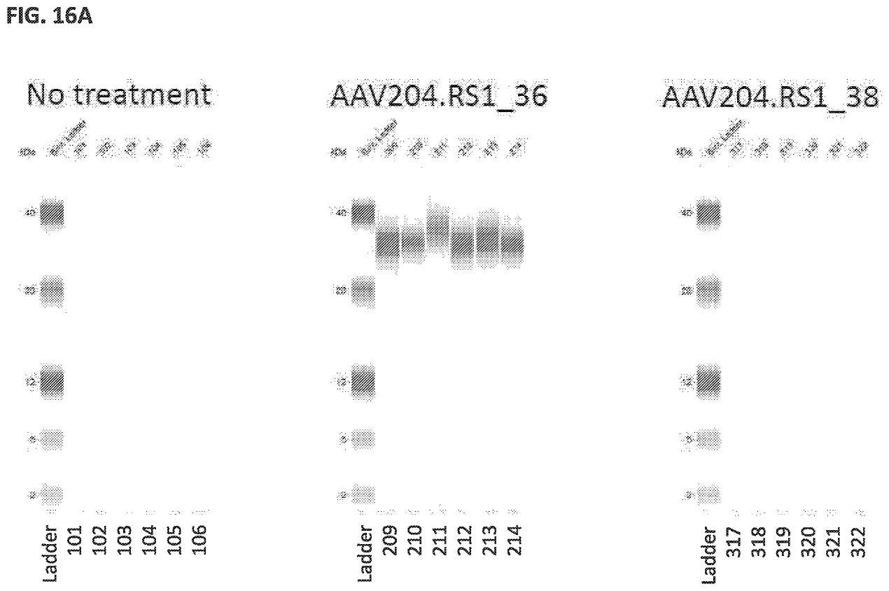

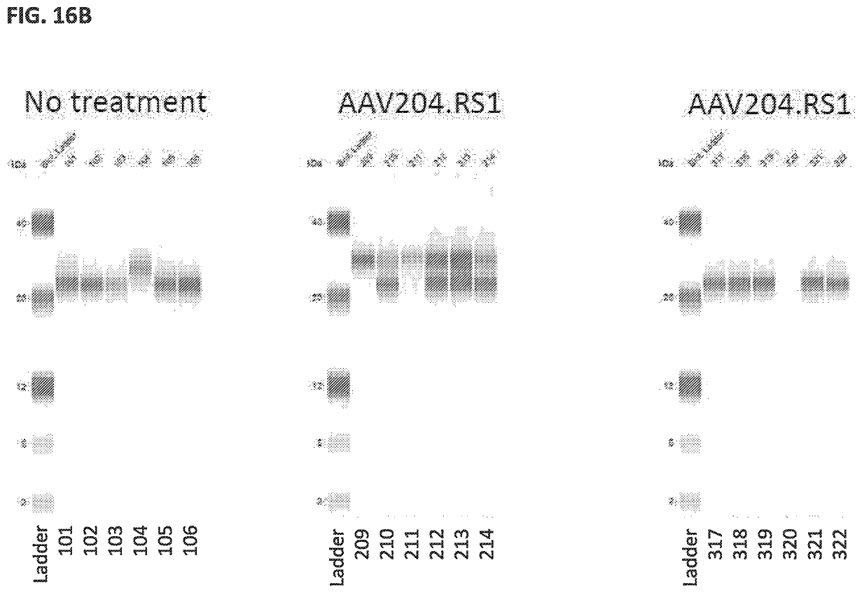

In the next study, the eye cups of wildtype mice were administered with AAV204 viral vector comprising either pA-RS1_36 or pA-RS1_38 vector genome at a dosage of 3e+9 vg/eye via intravitreal injection. At 30-day post-injection, samples were collected, and total protein extract were examined by Western analysis using anti-myc antibody (FIG. 16A). Myc-hRS1 protein expression was detected in all eyes administered with AAV204.pA-RS1_36 (comprising CBh promoter). In contrast, no myc-RS1 protein was detected in the treatment group administered with AAV204.pA-RS1_38 (comprising RK promoter) or in untreated control. This result was confirmed using anti-human RS1 (hRS1) antibody (FIG. 16B). This anti-hRS1 antibody in fact detected both human and mouse RS1 proteins, and the expression pattern of recombinant human RS1 (hRS1) based on anti-hRS1 antibody was consistent with that based on anti-myc antibody.

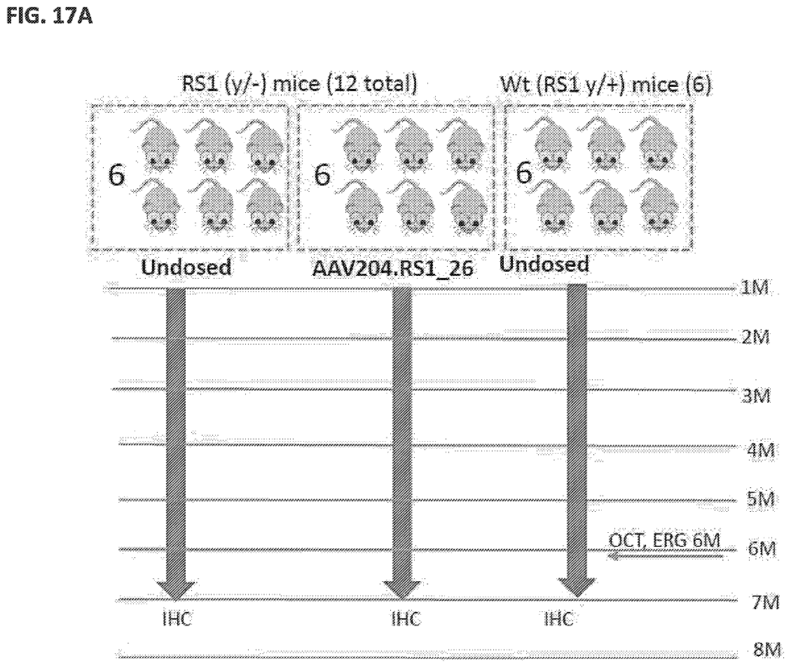

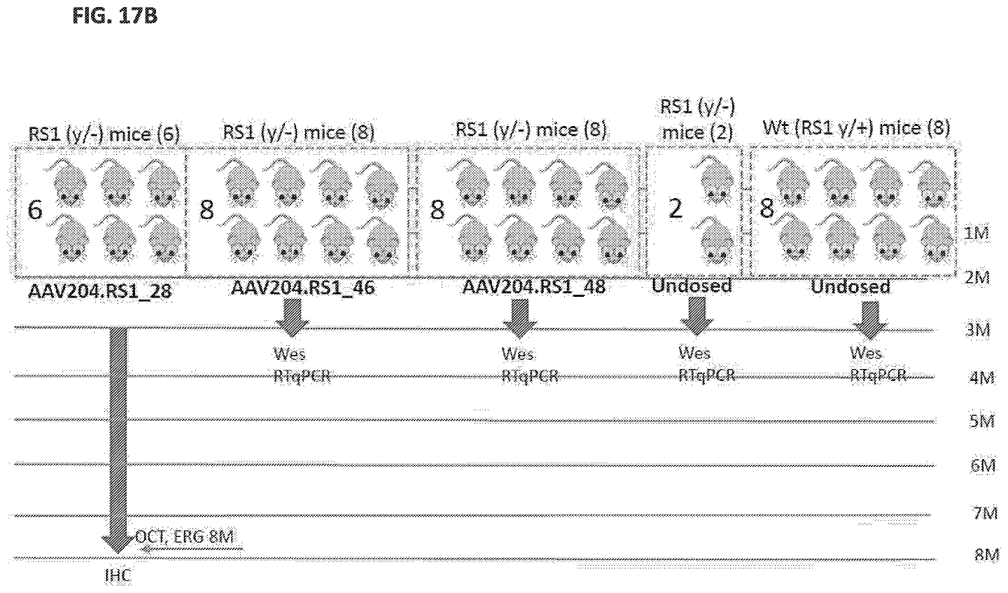

To assess ocular expression (FIG. 17A). the eye cups of RS1 (y/−) mice are administered with AAV204 viral vector comprising pA-RS1_26 vector genome. The efficacy of the treatment group is compared with undosed control groups (either RS1 (y/−) mice or wildtype RS1 (y/+) mice) at 6-month time point using optical coherence tomography (OCT) and electroretinogram (ERG), and the expression level of RS1 is evaluated at 7-month time point using IHC.

In another proof-of-concept study (FIG. 17B), three mice groups are administered with AAV204 viral vector comprising pA-RS1_28, pA-RS1_46, or pA-RS1_48 vector genome, respectively, and compared with the undosed control groups. The expression level of RS1 is evaluated at 3-month time point using Western analysis or RT-qPCR, or at 8-month time point using IHC. The efficacy of the treatment is evaluated at 8-month time point using OCT and ERG.

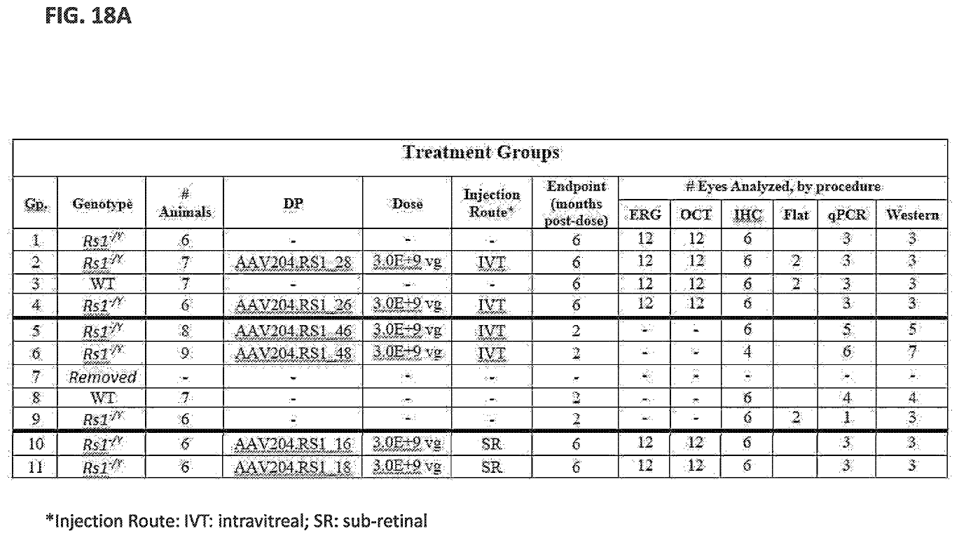

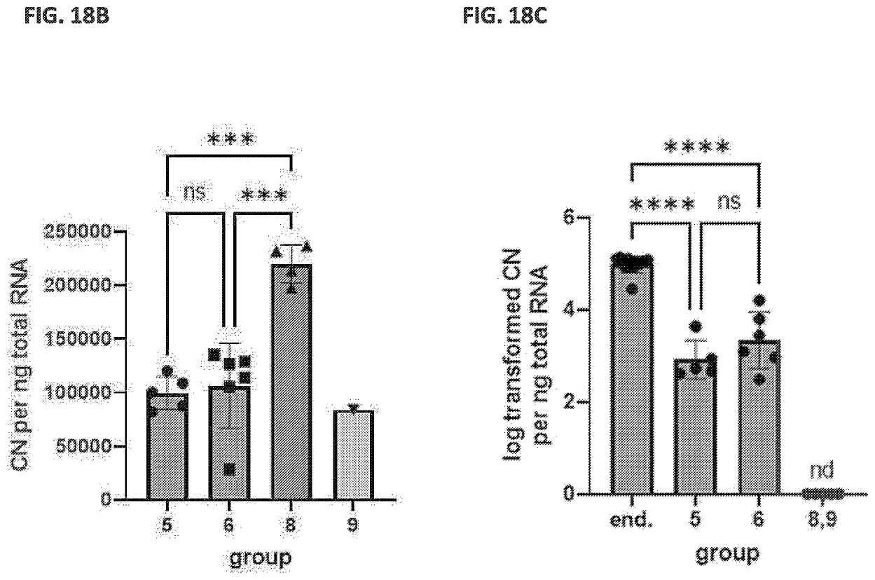

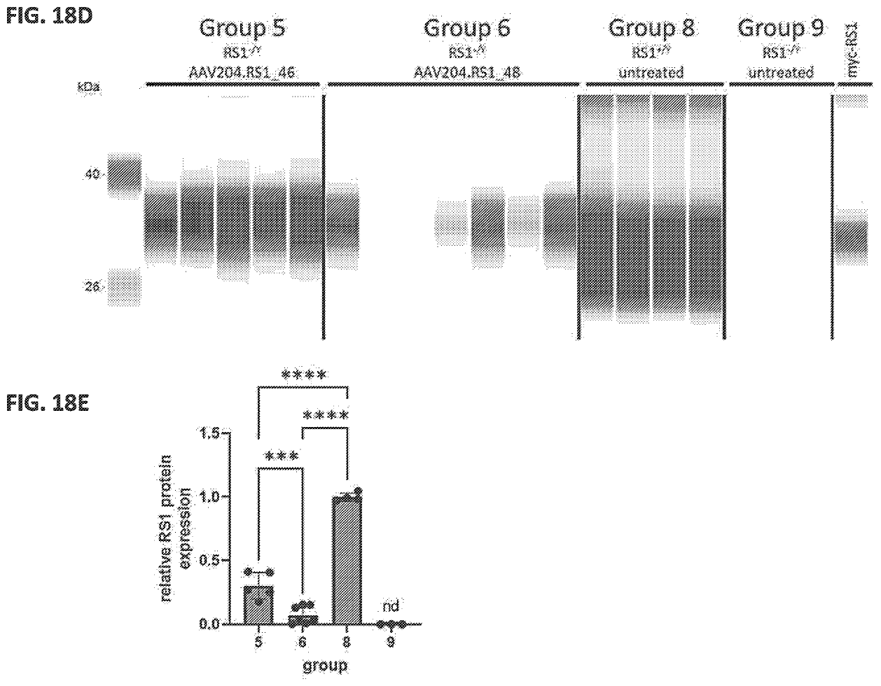

In another proof-of-concept study, male RS1 (−/Y) mice and wildtype male siblings were enrolled at 21±3 days of age and acclimated to the study for at least 3 days prior to dosing. The treatment groups were designed according to FIG. 18A. Briefly,
Groups 5-9 were used to demonstrate the in vivo infectivity of the intravitreally delivered vectors and to analyze the distribution of the viral-derived protein within the retina at 2 months post-treatment (mpt) timepoint. To facilitate protein detection, constructs having a N-term myc tag terminus were used (pA-RS1_46, pA-RS1_48).
Groups 1-4 were used to confirm expression longevity, as well as to analyze the effect of transgene expression on disease progression, at 6-month timepoint post intravitreal injection. The RS1 transgene used in these vectors did not have the myc tag.
Groups 10-11 were used to evaluate the effect of sub-retinal injection as an alternative drug delivery procedure, at 6-month timepoint post intravitreal injection, and were directly compared to the IVT injections done in parallel. Subretinal injection for the treatment of retinoschisis may carry more risk, as this is a more invasive technique that could lead to increased rates of retinal detachment given the structurally compromised state of the diseased retina. However, this has not been tested directly. The AAV viral constructs used in these two groups did not contain the myc tag encoding sequence or the S/MAR.
   “Eye cups” were prepared and then the neural retina was separated from the pigmented retinal pigment epithelium (RPE) to create retinal dissection samples.

### Analysis of Retinal Samples 2-Month Post-Intravitreal Delivery

Using primers directed against mouse RS1 (mRs1), endogenous gene expression was measured across all groups at the 2 mpt endpoint. mRs1 expression was significantly decreased in all mutant animals compared to WT, regardless of treatment (FIG. 18B). This is most likely a result of the degenerative state of the mutant retina, and in particular the loss of photoreceptors, where mRs1 is highly expressed. In the untreated mutant group (group 9), the mutant retina had similarly decreased mRs1 expression, suggesting that treatment did not affect endogenous mRs1 expression.

Viral-derived RS1 transcripts were then detected and differentiated from endogenous transcripts using primers targeted against the mycRS1 transgene (FIG. 18C). While mycRS1 was undetectable in untreated retinas (groups 8, 9), it was readily detected in all treated retinas (groups 5, 6). The promoter used did not have a significant effect on the total amount of virus-derived transcript. Transgenic myc-RS1 expression was nearly 2 log units lower than that of endogenous Rs1 in mutant animals.

Both endogenous and viral-derived proteins were detected simultaneously using the RS1-specific monoclonal antibody 3R10 by Western analysis to measure overall RS1 protein expression levels (FIGS. 18D and 18E). While expression varied between individual retinas, mean RS1 protein expression driven by the CBh promoter (group 5) was 30% of the RS1 produced in WT animals (group 8), and that driven by the RK promoter (group 6) was 7% of WT. The difference in the size of the RS1 protein between the treated and WT groups was due to the presence of the myc tag. No RS1 was detected in untreated mutant animals (group 9).

Immunohistochemical staining of additional eyes at the 2 mpt timepoint using the 3R10 antibody was performed to confirm transgene expression and evaluate protein localization. In addition, lectin PNA was used to label the outer segments of cone photoreceptors, and Iba1 was used to label retinal microglia. Low-magnification images of the entire retina illustrate the extent of transduction, with higher magnification images of the boxed regions to illustrate PNA and Iba1 staining as well as the distribution of RS1 across retinal layers. In some cases, multiple boxes regions are included to allow direct comparison of transduced and non-transduced areas of the same section.

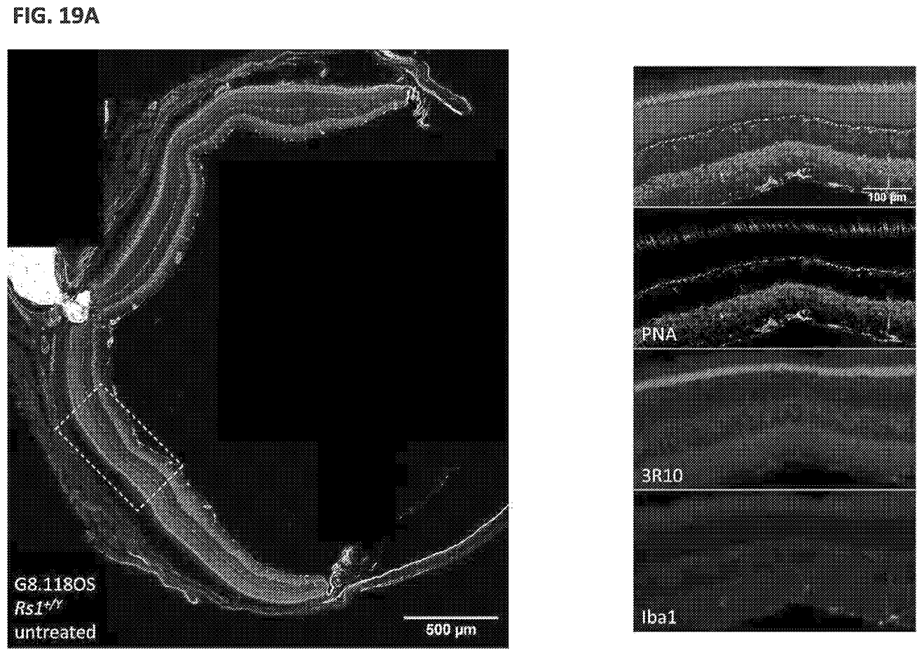

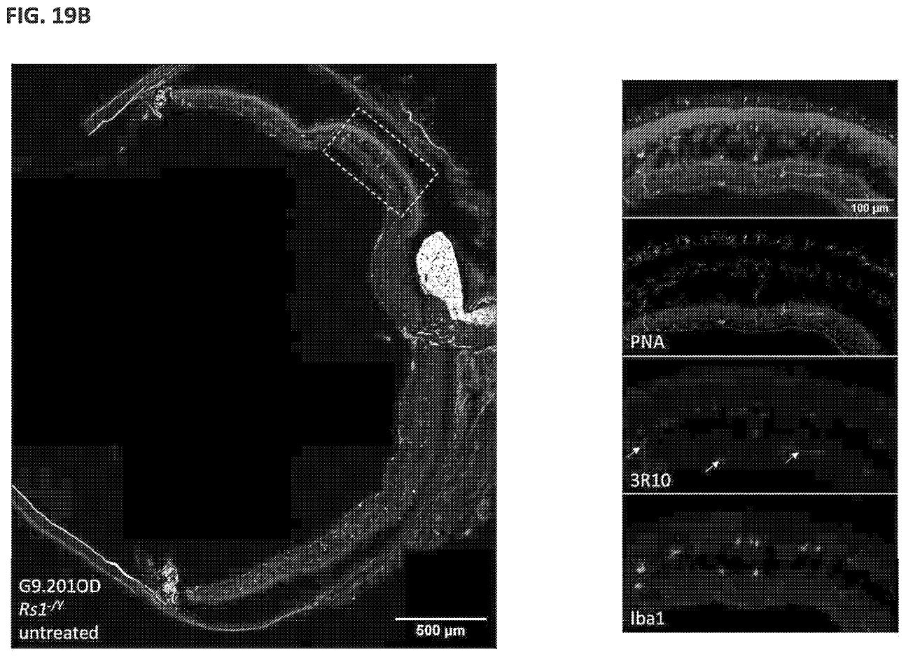

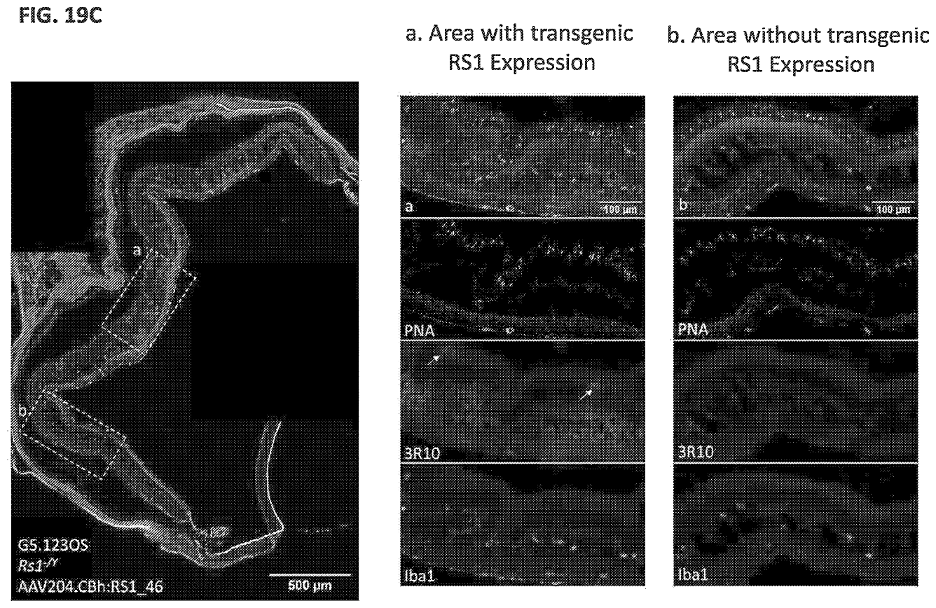

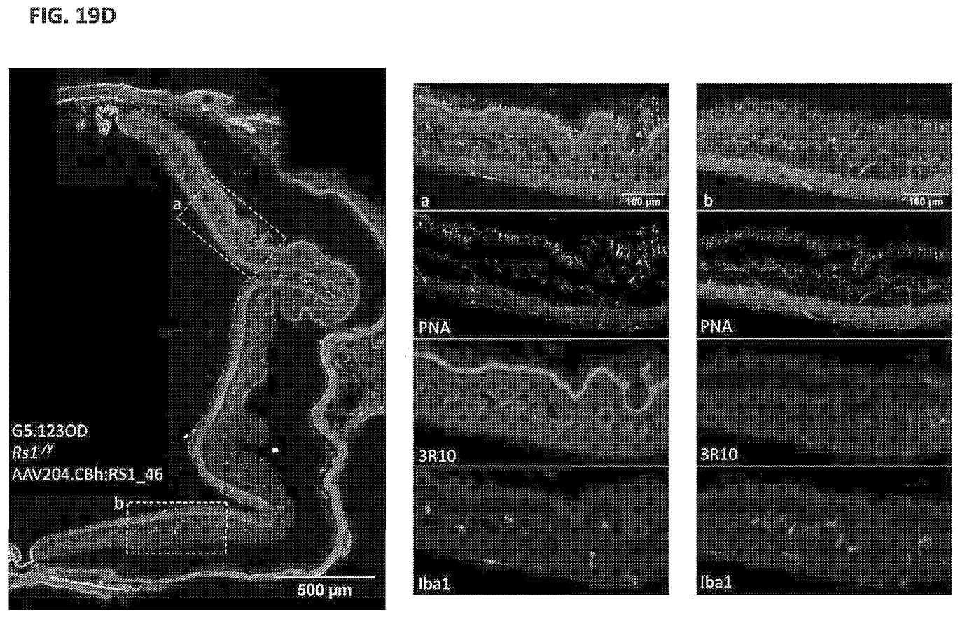

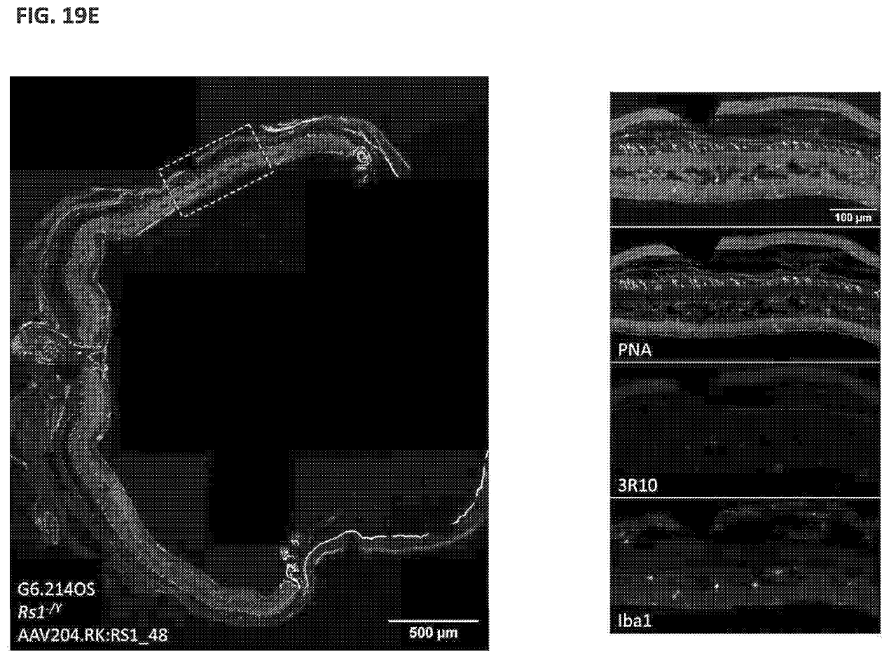

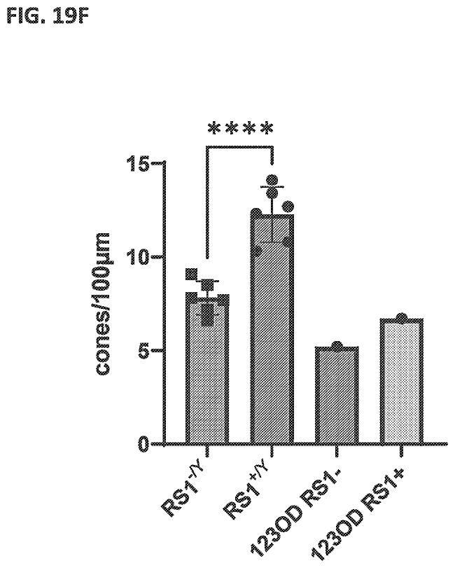

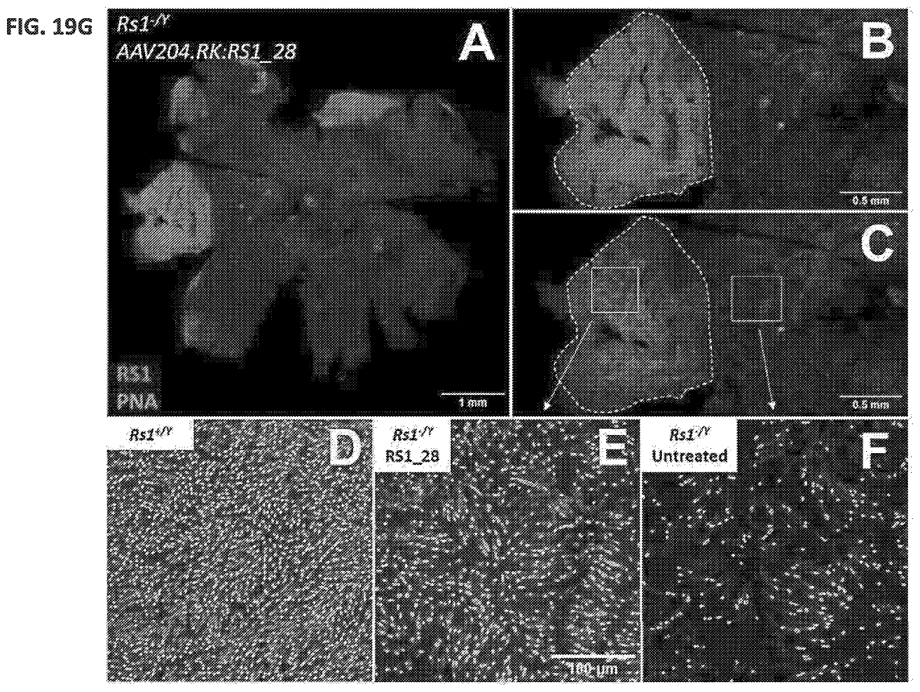

In WT animals from group 8, Rs1 localized prominently to the inner segments of photoreceptors, as well as more diffusely to the synaptic layers of the retina. Cone photoreceptors were regularly spaced through the length of the retina (12.2±1.5 cones per 100 μm), and Iba1 staining was minimal (FIG. 19A). In contrast, untreated mutant retinas (group 9) had no detectable RS1 staining, the cone density decreased (7.8±0.9 cones per 100 μm), and Iba1 staining was more prominent, indicating a hyperinflammatory state that is common in retinal diseases (FIG. 19B). Small, bright foci that appeared with RS1 staining in retinas that did not express RS1 were IgG-containing retinal blood vessels that became labeled with the anti-mouse IgG secondary antibody in the absence of RS1 expression. Several examples of these are indicated with arrows in FIG. 19B.

RS1 labeling in mutant retinas treated with AAV204.CBh:RS1_46 (group 5) was generally confined to the inner nuclear layer (INL), with small accompanying spots of labeled photoreceptor inner segments. There were no examples of labeled photoreceptors without adjacent inner retinal labeling. Cone density and Iba1 labeling were similar to that of untreated retinas, regardless of RS1 expression. A representative retina from this treatment group is shown in FIG. 19C. Arrows indicate small areas of transgene expression in photoreceptors. The right eye from one of the animals (#123) was exceptional in that transgene expression was observed along 58% of the retina, including the entire dorsal half, in both the inner retina as well as in photoreceptors (FIG. 19D); however, there were still no obvious changes to cone density or Iba1 labeling in this eye sample.

In contrast, none of the animals treated with AAV204.RK:RS1_48 (group 6) had observable RS1 immunolabeling (FIG. 19E). Cone density and Iba1 labeling in these animals was indistinguishable from untreated mutants. This was unexpected given that viral expression was detected by RT-PCR and Western analysis from other animals in this group.

Cone density was then quantified from IHC images of WT and untreated mutant animals, as well as the right eye from animal #123, which had a high level of transgene expression in photoreceptors. Cone density in mutant eyes was reduced to 63% of that in WT animals, as expected (FIG. 19F). Cone density was also measured in the well-defined RS1-positive and RS1-negative regions of eye #123OD (group 5). The entirety of this eye had lower cone density than the WT average, but there was a modest improvement in the RS1-positive area of that eye when compared with the RS1-negative area from the same eye.

### Analysis of Retinal Samples 6-Month Post-Intravitreal Delivery

To demonstrate the extent of transduction in IVT-injected eyes, the retinas from one animal treated with AAV204.RK:RS1_28 (group 2) were stained as flat mounts (FIG. 19G). Transgene expression was restricted to a well-defined area in the periphery, covering about 10% of the entire retina. The extent of transduction was similar in the fellow eye. The cone density in a non-transduced part of the retina was about 19% of that in a similarly prepared WT control. In the transduced part of the retina, cone density was improved to 46% of WT. The results showed that expression of the RS1 transgene was more limited than expected and concentrated near the injection site in the dorsal retina.

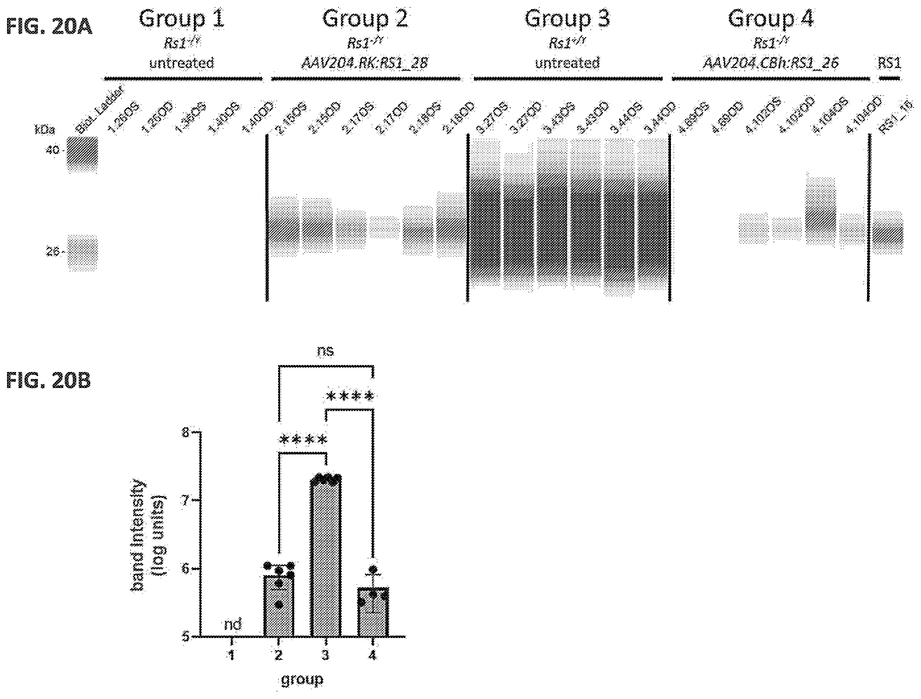

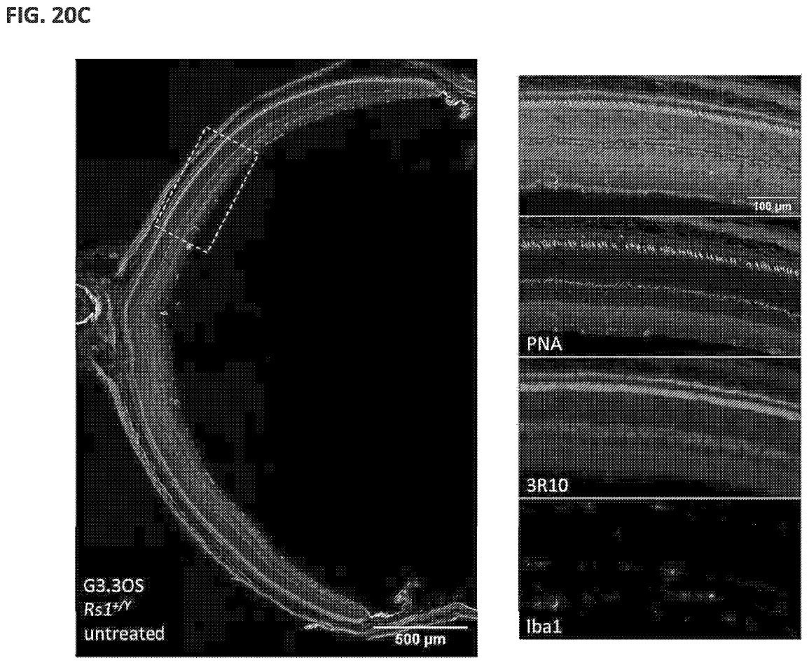

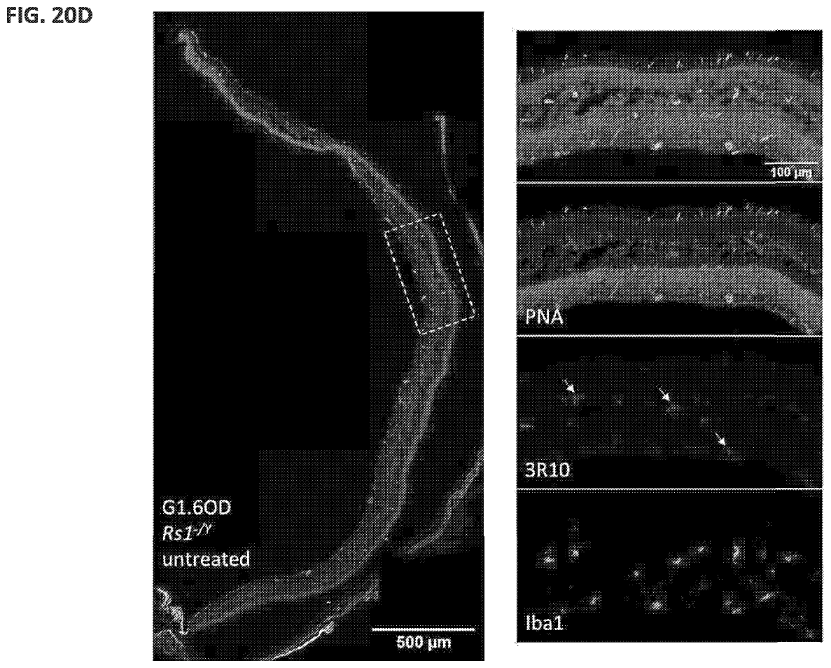

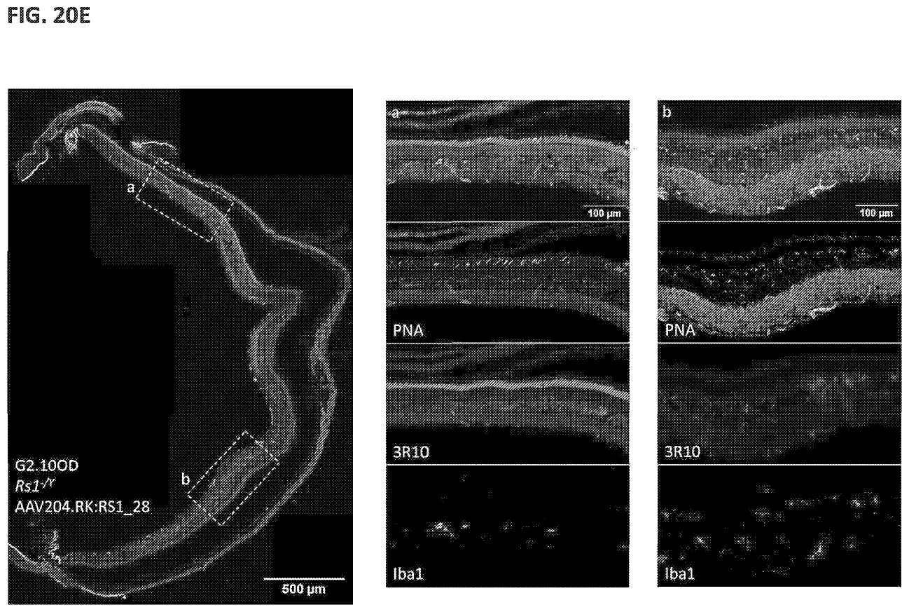

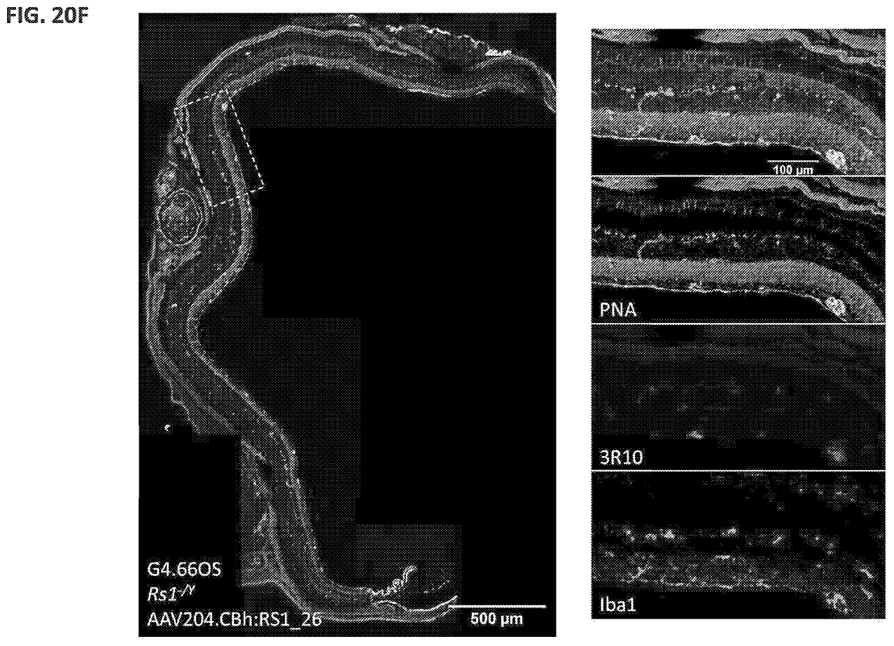

Western analysis was performed on retinas harvested at the 6 mpt timepoint (FIGS. 20A and 20B). Using the RS1-specific antibody 3R10, RS1 expression was detected in mutant eyes treated with either AAV204.CBh:RS1_28 (group 2) or AAV204.RK:RS1_26 (group 4). Among all treated animals, the level of RS1 expression ranged from 1.5% to 5.5% of that detected in WT retinas. Unexpectedly, expression from the CBh promoter-controlled transgene was not significantly higher than that from the RK promoter-controlled transgene. No expression was detected in untreated mutant animals.

Immunohistochemistry (IHC) was then used to examine the distribution of RS1 expression in treated eyes at this timepoint and to determine the effect of RS1 expression on the disease phenotype. Similar to the previous 2 mpt timepoint, RS1 expression in WT animals was concentrated in photoreceptor inner segments with more diffuse expression in the inner retina (FIG. 20C). In contrast, no expression was seen in untreated mutant animals (FIG. 20D). Labeling of blood vessels with the anti-mouse secondary antibody was seen again in the mutant retinas (FIG. 20D, examples indicated with arrows). Cone density was likewise reduced in the mutants at this timepoint, and Iba1 labeling was more prominent.

In animals from group 2 treated with AAV204.RK:RS1_28 that had detectable RS1 expression, it ranged from 14% to 59% of the section. This should not be construed as a percentage of the entire retina, as only the sections with maximal expression were imaged. A representative example of this group is shown in FIG. 20E. RS1 labeling was not restricted primarily to the inner retina, but most transduced areas included considerable photoreceptor expression as well. In contrast, none of the group 4 eyes treated with AAV204.CBh:RS1_26 had detectable expression (FIG. 20F).

Notably, the localization of recombinant RS1 in the retina of group 2 animals was identical to that of the endogenous protein, with a significant amount of staining in photoreceptor inner segments and more diffuse staining throughout the inner retina, even in animals treated with a photoreceptor-specific promoter. This indicates that secreted RS1 is capable of radial diffusion through the retina to reach receptors in adjacent layers.

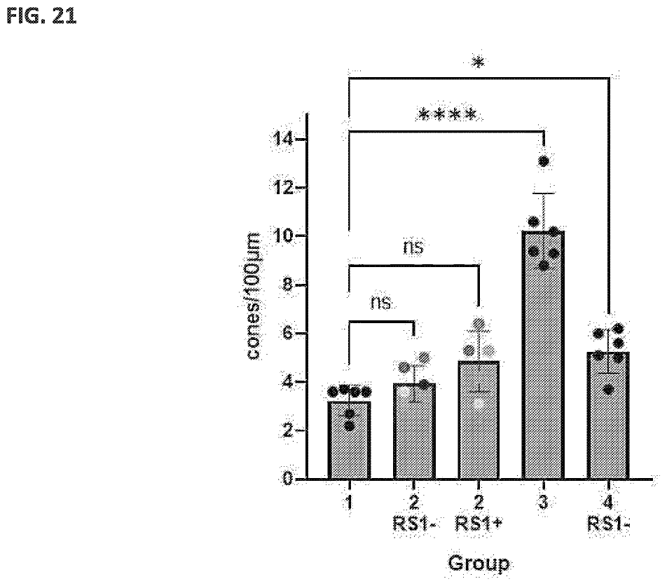

Cone density was quantified in all animals at the 6-month timepoint (FIG. 21). In mutants treated with AAV204.RK:RS1_28 (group 2), the RS1-positive and RS1-negative regions were treated as separate data points. Cone density in untreated mutants was 31±6% of wild-type, and the mean density in group 2 was unchanged, regardless of RS1 expression. However, in all but one of the group 2 animals, the cone density was higher in the RS1-positive region compared to the adjacent RS1-negative region in the same section. This improvement ranged from 15% to 71%. Surprisingly, group 4 animals treated with AAV204CBh:RS1_26 had a mean cone density that was 63% greater than the untreated group (p=0.0217), despite having no observable RS1 expression. Thus, cone density was improved after treatment with either viral construct, despite that in the case of RK-promoter controlled RS1 such improvement was not correlated with observable RS1 immunoreactivity.

Optical coherence tomography (OCT) was then used to analyze the retinal status. OCT is a noninvasive imaging technique that generates cross-sectional images of the retina and is regularly used to aid in the diagnosis of retinal diseases. This is a particularly useful technique for retinoschisis, as the large retinal cavities that are characteristic of the disease are easily observed. However, by 7 months of age most of the schises are resolved in RS1 mutant mice. OCT images are similar to histological sections, but they are more limited in that they cannot be directly correlated with expression data. Therefore, interpretation of these images must include the assumption that expression of the transgene is widespread, or at least that it overlaps with the OCT field.

Analysis of the OCT data from this study revealed that the schisis phenotype was apparent only in about one third of the imaged mutant eyes, regardless of treatment. This is consistent with that feature of the disease beginning to resolve around this timepoint in mutant mice. No schises were observed in WT eyes.

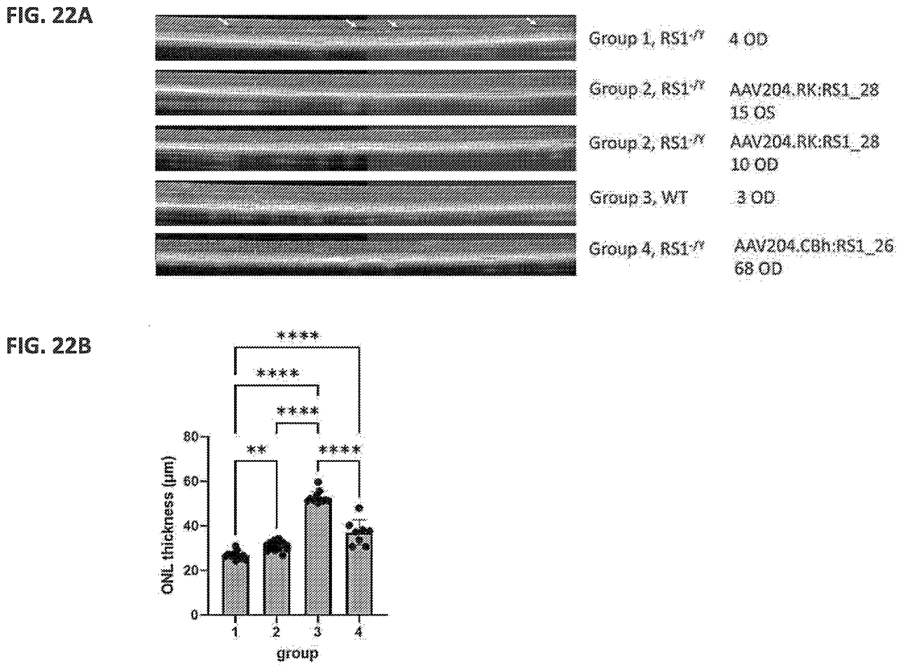

Representative OCT images are shown in FIG. 22A, including the two treated eyes from Group 2 in which maximal RS1 expression was observed by either IHC or Western analysis. Each image is of the dorsal retina, in the mid-periphery. In 10OD, this is approximately the area in which strong RS1 expression was observed by IHC. Untreated mutant eyes from group 1 had a significantly thinner Outer Nuclear Layer (ONL) than WT eyes from group 3 (26.9±1.9 μm vs. 52.8±2.6 μm). Eyes from both treatment groups had a thicker ONL than the untreated animals, but they remained thinner than WT eyes (FIG. 22B).

The current study was completed using isolated retinas instead of whole eyes. Isolating the retina has the effect of concentrating the target tissue within the sample and making the limited amount of expression from the RK promoter visible. Furthermore, using isolated retina excludes the extra-retinal expression that occurs with the ubiquitous CBh promoter, providing a clear indication of the RS1 expression level in the target tissue. The results show that (i) RS1 expression from either promoter was readily detectable in treated mutant eyes, regardless of the promoter used; and (ii) unexpectedly, the CBh-driven RS1 expression in the retina at 2 months post-treatment is nearly 1.5 log units lower than that of endogenous RS1.

### Analysis of Retinal Samples 6-Month Post-Sub-Retinal Delivery

In addition to intravitreal injection, selected AAV viral vectors were also delivered by sub-retinal injection (groups 10 and 11 in FIG. 18A). The corresponding samples were analyzed together with samples from the control groups 1 and 3 (untreated mutant, and wildtype, respectively), at 6 mpt timepoint.

The RS1-specific monoclonal antibody 3R10 was used to confirm transgene expression and evaluate protein localization. In addition, lectin PNA was used to label the outer segments of cone photoreceptors, and Iba1 was used to label retinal microglia. Low-magnification images of the entire retina are shown to illustrate the extent of transduction, with higher magnification images of the boxed regions to illustrate PNA and Iba1 staining as well as the distribution of RS1 across retinal layers. In some cases, multiple boxes regions are included to allow direct comparison of transduced and non-transduced areas of the same section.

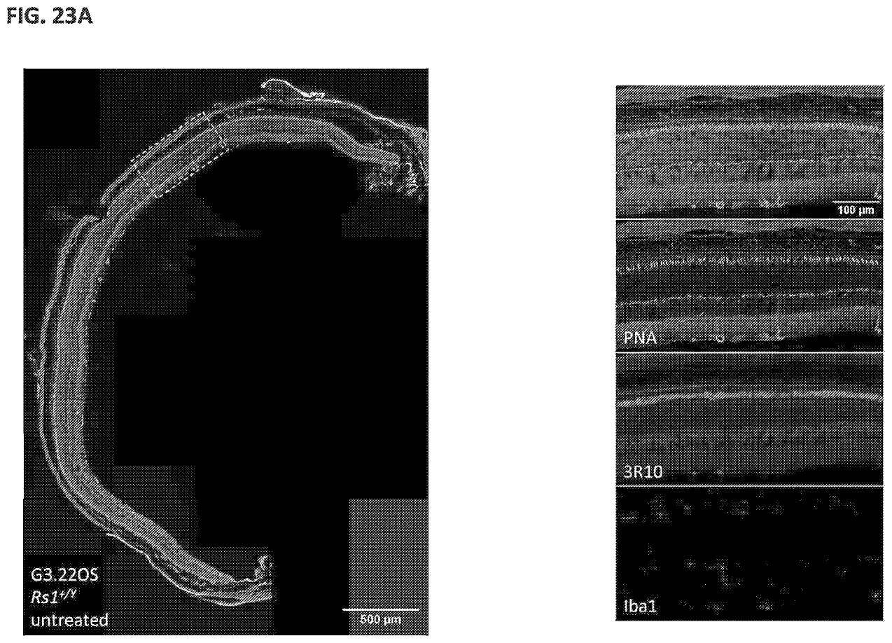

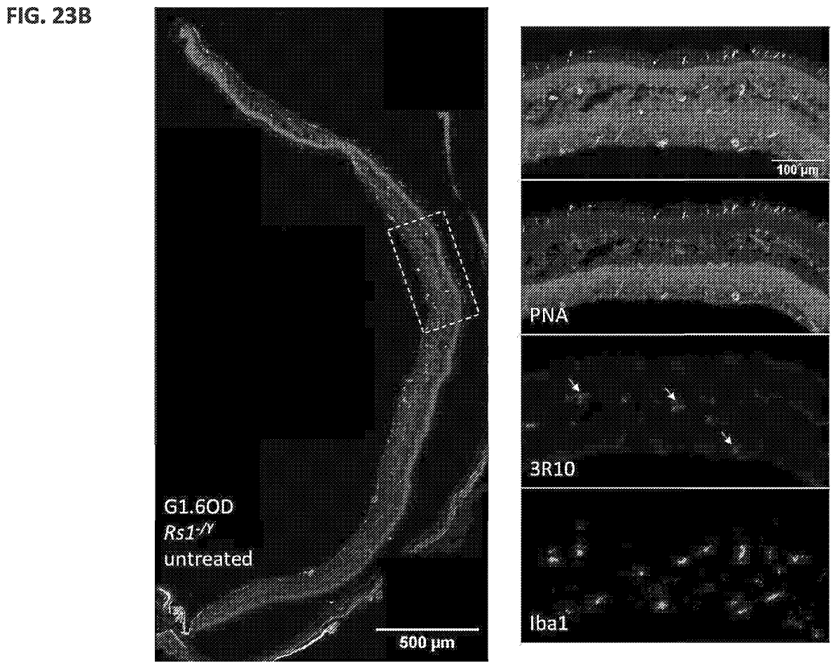

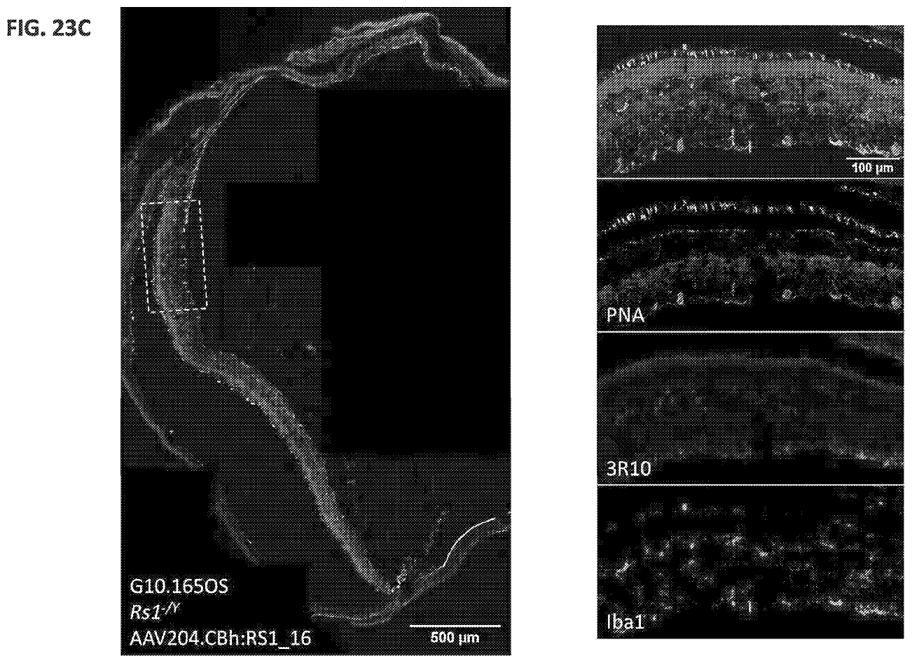

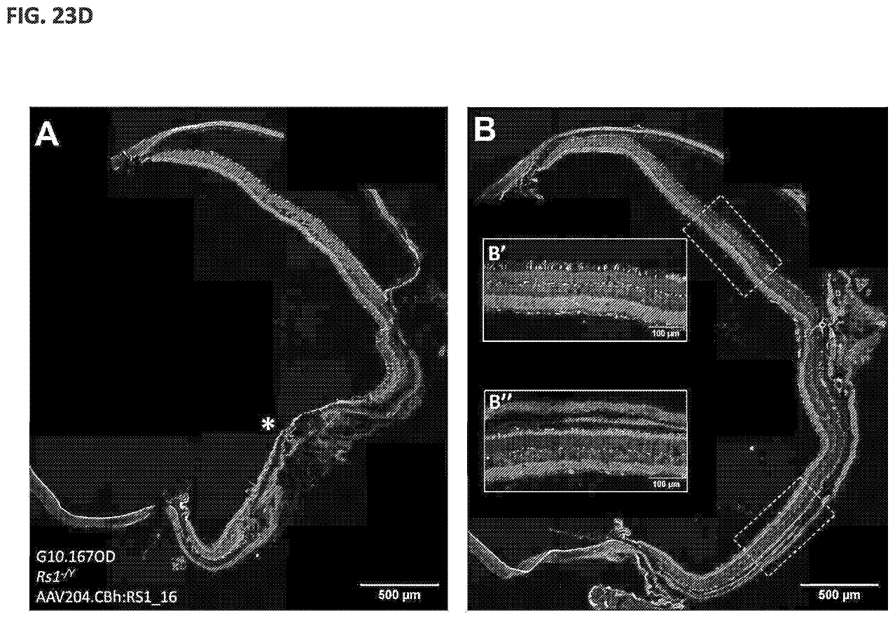

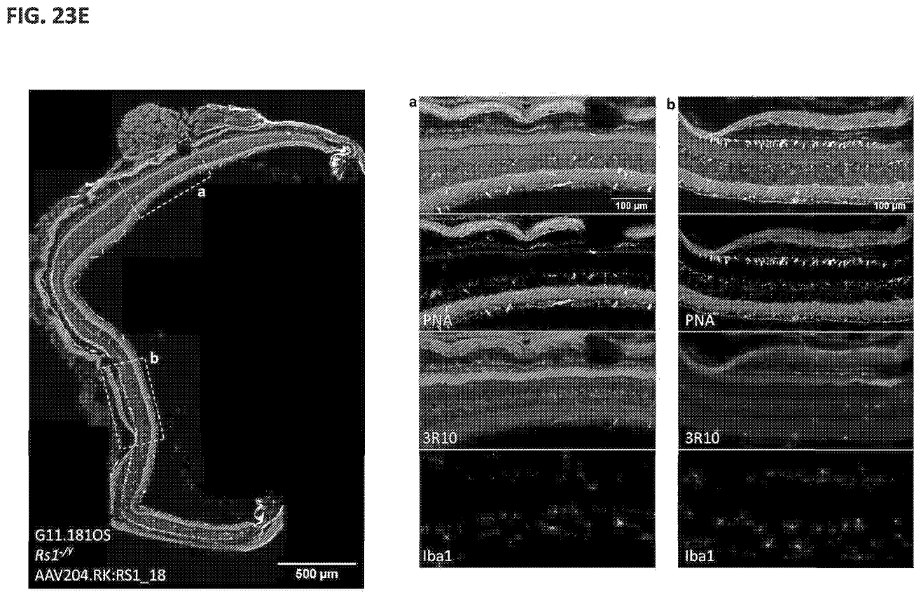

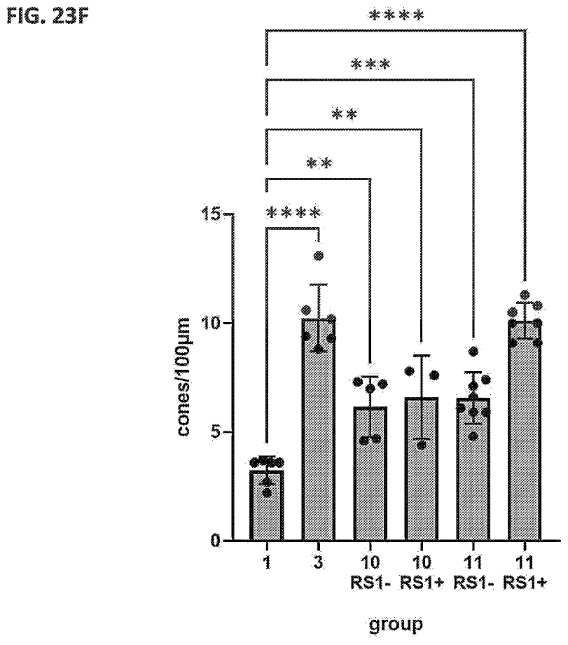

In WT animals from group 3, endogenous Rs1 localizes prominently to the inner segments of photoreceptors, as well as more diffusely to the synaptic layers of the retina. Cone photoreceptors are regularly spaced through the length of the retina (10.2±1.6 cones per 100 μm), and Iba1 staining is minimal (FIG. 23A).

In contrast, untreated mutant retinas (group 1) had no detectable RS1 staining, the cone density decreased (3.2±0.6 cones per 100 μm), and Iba1 staining was more prominent, indicating a hyperinflammatory state that is common in retinal diseases (FIG. 23B). Small, bright foci that appear with RS1 staining in retinas that do not express RS1 are IgG-containing retinal blood vessels that become labeled with the anti-mouse IgG secondary antibody in the absence of RS1 expression. Several examples of these are indicated with arrows in FIG. 23B.

Mutant animals treated with AAV204.CBh:RS1_16 (group 10) exhibited severe retinal degeneration associated with the injection site, with no remaining retinal cells (FIG. 23C). In some cases, there were adjacent sections of retina with RS1 expression, although cone density in these RS1+ areas was not improved. However, the mean cone density in the RS1-areas of these retinas, which did not suffer injection-related degeneration, was approximately double that of untreated animals (6.2±1.4 vs. 3.2±0.6 cones per 100 μm, respectively), suggesting that RS1 expression below the level of IHC detectability may extend beyond the bleb and still provide some therapeutic benefit. No change was seen in Iba1 staining of treated animals. The right eye from one animal (#167) was unique in that it had a major lesion in the ventral retina, most likely a mechanical injury sustained during injection. Despite that, uniform RS1 staining was seen throughout the inner retina, as well as in photoreceptors adjacent to the lesion (FIG. 23D). It is likely that this lesion compromised physical barriers that normally inhibit the diffusion of viral particles through the retina, permitting more widespread expression.

In contrast, seven of eight eyes treated with AAV204.RK:RS1_18 (group 11, FIG. 23E) had very significant RS1 expression in photoreceptors, and only one of those seven animals had the severe degeneration that was common in animals treated with CBh:RS1. Expression was generally localized to the dorsal retina at the bleb. The RK promoter is rod-specific, but labeling was also observed in the inner retina adjacent to the RS1+ photoreceptors in all seven eyes, indicating that RS1 produced in the outer retina can diffuse radially. In four of the seven eyes, a cone-depleted area was associated with the central bleb, although the rest of the retina was intact. Outside of this cone-depleted area, there was an RS1-positive margin in which the cone density was actually increased compared to the RS1-negative part of the same section. In fact, this marginal area had a cone density that was equivalent to a wild-type eye. Taken together, these results suggest that extreme overexpression of RS1 in mutant animals may be severely retinotoxic and that moderate overexpression is detrimental to cones, but that appropriate dosing can yield therapeutic results.

The chart in FIG. 23F summarizes the cone density measurements. Areas of severe degeneration were omitted. For group 11 eyes treated with RK:RS1, the cone-depleted RS1-positive zone was omitted. Group 11 RS1+ is therefore representative of the cone-enriched area surrounding the depleted zone. All treated eyes had a higher cone density than untreated control eyes, even in areas lacking visible RS1 expression. Furthermore, the cone-enriched, RS1-positive area in group 11 is indistinguishable from WT

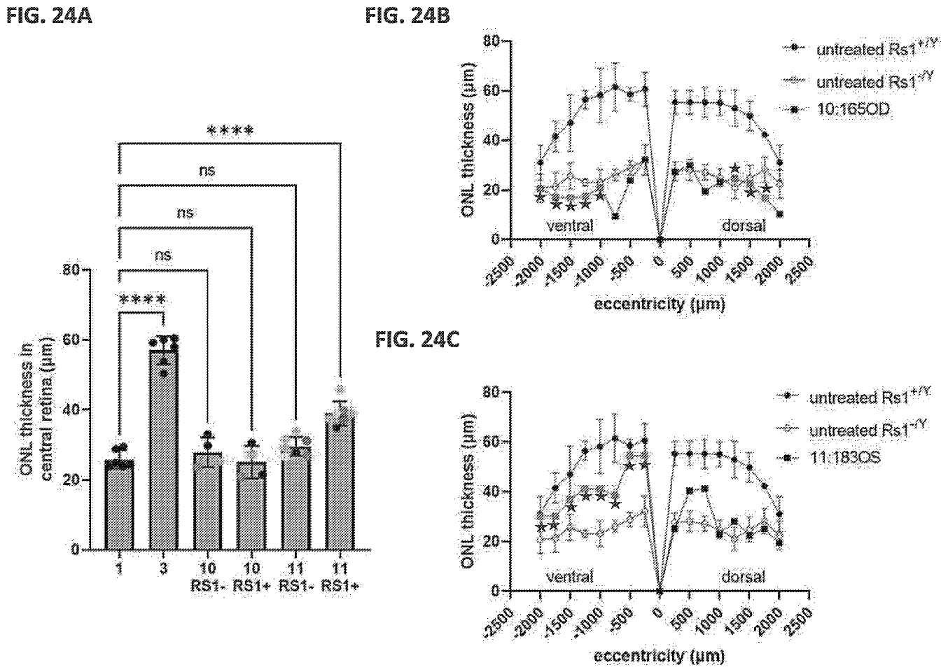

The thickness of the outer nuclear layer (ONL) was also measured from DAPI-stained frozen sections (FIGS. 24A-24C). For this analysis, areas of severe degeneration were omitted. In group 10 animals treated with CBh:RS1, ONL thickness was equal to untreated mutant animals. In all group 11 animals treated with RK:RS1, the ONL was thicker specifically in areas of the retina with RS1 expression. In areas of those eyes that did not express RS1, the ONL thickness was roughly equal to that of untreated mutants.

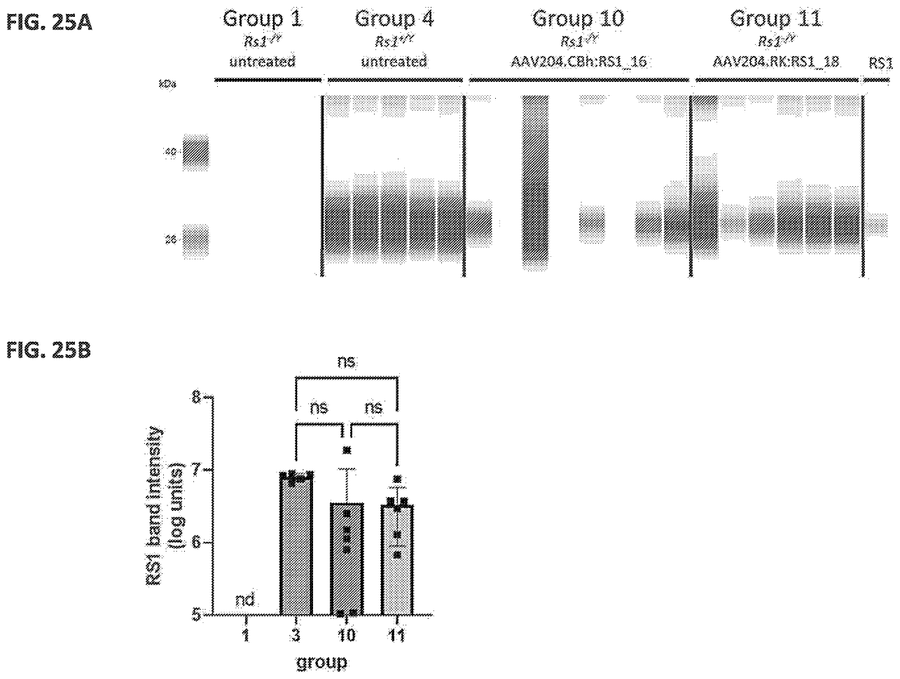

Protein Analysis: Western analysis was performed on frozen tissue harvested 6 months post-treatment (FIGS. 25A and 25B). Using the RS1-specific antibody 3E10, RS1 expression was detected in mutant eyes treated with either AAV204.CBh:RS1_16 (group 10) or AAV204.RK:RS1_18 (group 11). Expression of recombinant RS1 in both groups was statistically equal to that of endogenous RS1 expression in WT animals (group 3).

Optical coherence tomography (OCT): as mentioned above, interpretation of OCT images assumes that expression of the transgene is widespread, or at least that it overlaps with the OCT field. This is reasonable in this study because the injections were done subretinally, and the survey of IHC images from animals treated with RK:RS1_18 (group 11) suggests that expression was widespread through the dorsal retina and excluded from the ventral side.

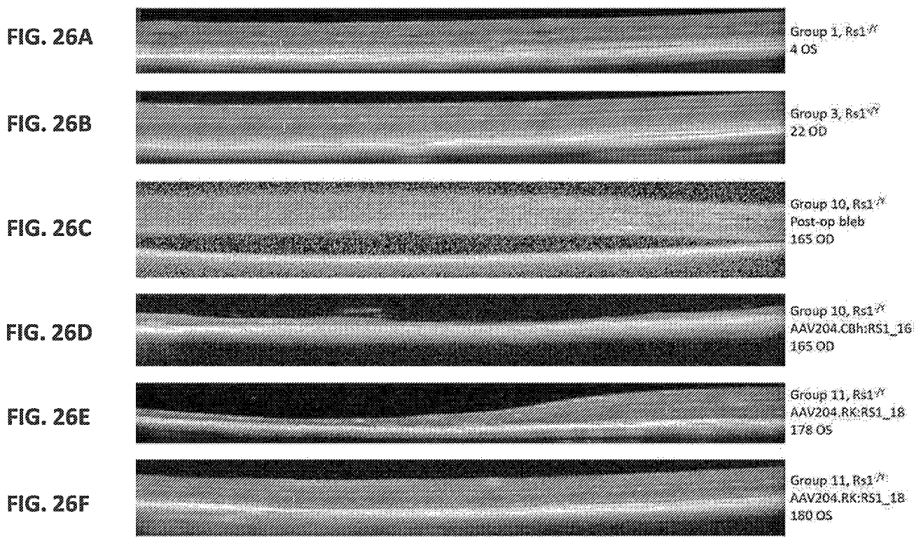

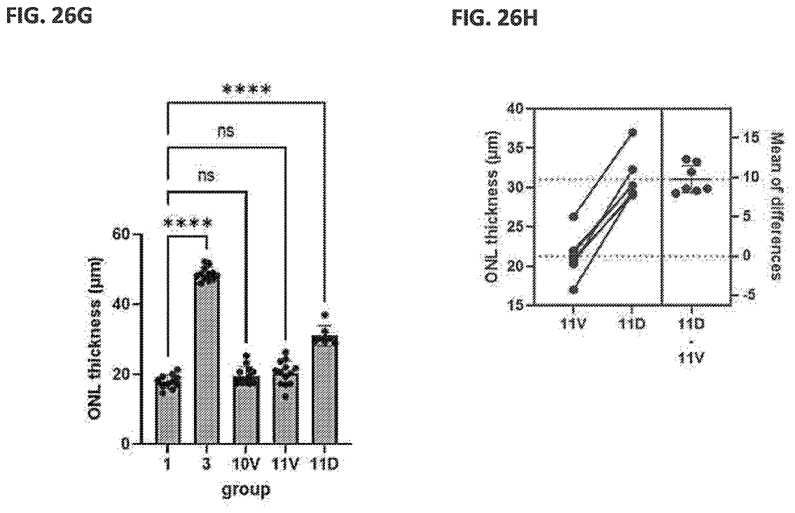

OCT data are shown in FIGS. 26A-26F. The ONL of untreated mutant eyes was nearly one-third that of wild-type eyes (17.9±1.9 vs 48.7±1.9 μm, FIGS. 26A-26B). All treated eyes were imaged immediately after injection to confirm the presence of a bleb (FIG. 26C). All 13 eyes successfully injected with CBh:RS1_16 showed severe degeneration (FIG. 26D). Several examples overlapped with the margin of the bleb in which some retina remained, but the ONL thickness there as well as that of the ventral, untreated retina was not significantly different from uninjected mutant animals.

In contrast, among the eyes treated with RK:RS1_18 (group 11), only seven of the 13 eyes with interpretable images had signs of severe injection-associated degeneration, but four of the seven still had measurable areas in the margin of the bleb (FIG. 26E-26F). Among the six non-degenerated eyes and the four degenerated eyes with measurable margins, all of them showed improvement in the ONL thickness when compared with the corresponding ventral retina as well as the untreated mutant eyes from Group 1 (FIG. 26G-26H).

Electroretinography (ERG): Electroretinography is a non-invasive technique that measures retinal function in response to a light flash, and it can be modified to elicit either rod- or cone-based responses. The intensity of the flash stimulus can be varied as well, and the amplitude of the ERG components will respond in parallel. The dark-adapted (DA) ERG elicits a rod dominated response and is comprised of two main components: the a-wave, which is the initial negative amplitude peak and reflects rod photoreceptor activity; and the b-wave, which is a large positive amplitude peak and primarily reflects the activity of ON-bipolar cells. In light-adapted (LA) ERGs, a dim background light is used to desensitize the rod photoreceptors, allowing the cone response to be isolated. Cone responses can also be measured using a flicker paradigm, where the frequency of the stimulus is faster than the recovery time of rod photoreceptors. More description of ERG can be found, for example, in Georgiou, Anne L., et al., Current eye research 39.5 (2014): 472-486, the content of which is incorporated by reference herein in its entirety.

The ERG protocol used in this study utilizes a full-field stimulus, so the recordings reflect the net response across the entire visual field. The ability to resolve the effects of a rAAV-based treatment on the ERG is therefore dependent on the extent of transgene expression across the retina.

In early stages of retinoschisis disease, the synapses between photoreceptors and inner retinal neurons are disrupted. In a DA ERG, this manifests initially as a reduction in b-wave amplitude. As the disease progresses, photoreceptors are lost, and the a-wave amplitude decreases as a result. In severe cases the ERG response can be undetectable.

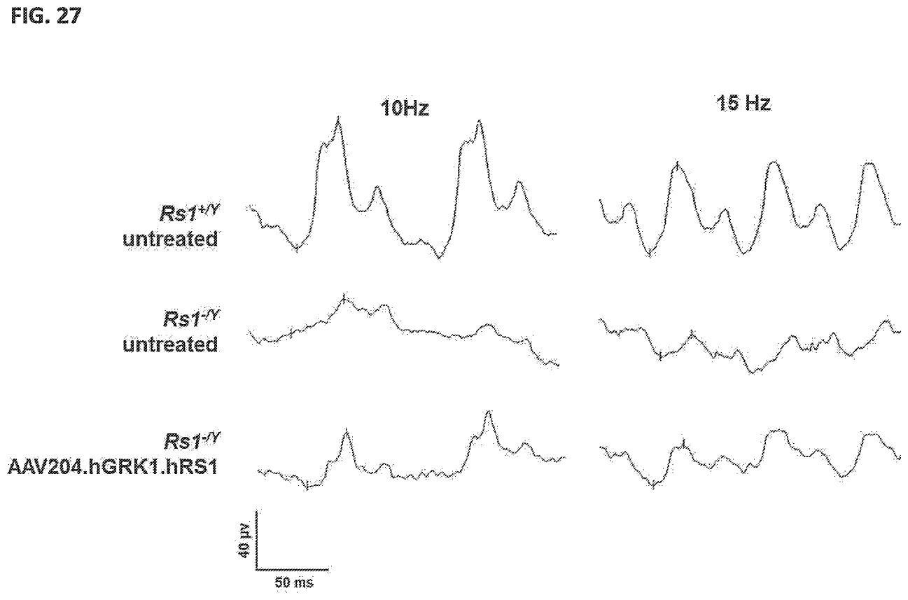

A summary of the ERG results is presented in Table 8. While no improvement was seen in treated animals after single-flash stimuli, flicker ERGs indicated a significant improvement in cone responses in group 11 animals treated with RK:RS1_18. Representative flicker ERG traces are shown in FIG. 27.

TABLE 8
ERG Results Matrix

| | | Group 1 | Group 3 | Group 10 | Group 11 |
|---|---|---|---|---|---|
| | | Genotype: | | | |
| | | Rs1^{-/Y} | WT | Rs1^{-/Y} | Rs1^{-/Y} |
| | | Treatment: | | | |
| | | — | — | CBh:RS1_16 | RK:RS1_18 |
| DA 0.01 | a-wave IT (ms) | n/a | | | |
| | a-wave amp (μV) | | | | |
| | b-wave IT (ms) | 85.1 ± 8.8 | 86.6 ± 4.3 | 82.8 ± 10.3 | 77.7 ± 12.4 |
| | b-wave amp (μV) | 140.3 ± 40.3 | 372 ± 76.1 | 105.7 ± 61.5 | 145.7 ± 76.7 |
| DA 3.0 | a-wave IT (ms) | 25.1 ± 5.7 | 19.2 ± 0.8 | 26.6 ± 4.3 | 19.5 ± 3.5 |
| | a-wave amp (μV) | 93.5 ± 19.1 | 239.8 ± 46.7 | 37.6 ± 32.2 | 86.4 ± 51 |
| | b-wave IT (ms) | 67.6 ± 10.3 | 70.1 ± 4.1 | 71.3 ± 14 | 69.5 ± 13.3 |
| | b-wave amp (μV) | 206.8 ± 49.8 | 560.4 ± 100 | 128.8 ± 52.7 | 229.2 ± 72 |
| | b/a ratio | 2.3 ± 0.5 | 2.3 ± 0.1 | 5.7 ± 5.1 | 3.5 ± 2.3 |
| LA 6.0 | a-wave IT (ms) | n/a | | | |
| | a-wave amp (μV) | | | | |
| | b-wave IT (ms) | 67.2 ± 22.8 | 46 ± 2.1 | 79.8 ± 22.9 | 53.4 ± 12.2 |
| | b-wave amp (μV) | 60.2 ± 70.1 | 148.8 ± 21.3 | 63.9 ± 29.3 | 76 ± 31.6 |
| 10 Hz | time to peak (ms) | 53.1 ± 6.7 | 43.5 ± 3.1 | 47.4 ± 11 | 48.3 ± 5.2 |
| flicker | T-P amp (μV) | 22 ± 9 | 87.4 ± 14.9 | 17.9 ± 7.4 | 37.1 ± 20.3 |
| 15 Hz | time to peak (ms) | 56.3 ± 6.5 | 39.6 ± 2.3 | 45.4 ± 13.3 | 48.6 ± 6.8 |
| flicker | T-P amp (μV) | 15.5 ± 5.3 | 69.1 ± 10.9 | 16 ± 12.3 | 29.5 ± 14.8 |

Summary of ERG results at the 6-month timepoint. All values are mean ± SD. IT = implicit time; T-P = trough-to-peak.

### Summary of Results

Results of the sub-retinal injection show that RS1 expression from AAV204.CBh:RS1_16 at the dose used in this study may result in certain toxicity, such as possible retinal degeneration at the injection site. CBh is a strong, ubiquitous promoter. It is possible that overexpression of RS1 in cells that do not normally express it may be the cause of the degeneration, suggesting that a lower dose may be used for delivery of RS1 transgene that is operably linked to a strong, ubiquitous promoter. In contrast, only about half of the eyes that were treated with AAV204.RK:RS1_18 had similar areas of degeneration. RK is a photoreceptor-specific promoter, which is the primary site of RS1 expression in the WT retina. Although Western analysis showed that the two promoters yielded equal amounts of recombinant protein, the exclusion of RS1 expression in non-photoreceptors was clearly beneficial. Even with RK-driven, photoreceptor-specific expression, secreted RS1 was observed in the inner retina adjacent to RS1-positive photoreceptors, indicating that it was capable of diffusing radially through the retina. This expression pattern closely matches that of endogenous RS1 expression in WT animals. Notably, Western analysis showed RS1 expression in treated eyes equal to that of endogenous expression in WT mice, even though only a fraction of the retina was treated, indicating that (i) the load of RS1 protein in the treated areas is likely to be significantly above the physiological level, and (ii) the degeneration associated with the RK promoter is likely to be resolved by using lower doses.

Because RS1 mutations cause severe structural damage to the retina, this study was initially designed for intravitreal injection, which is a less invasive technique than subretinal, and the intent was to minimize the risk of causing additional damage to an already compromised retina. However, animals treated intravitreally with AAV204.CBh:RS1_26 and AAV204.RK:RS1_28 had minimal expression of recombinant protein, which promoted the study with subretinal treatment. With subretinal delivery, the vector is restricted to a much smaller space immediately adjacent to the target layer, thereby increasing the transduction rate and boosting transgene expression. Indeed, comparison of these two studies demonstrates that subretinal injection yields superior expression to intravitreal.

Efficacy in this study was evaluated via three different measurements: cone density, ONL thickness, and ERG, all of which were decreased in untreated mutant mice. RS1 Expression from the CBh promoter caused catastrophic retinal degeneration in all treated animals. The RK promoter, with an expression profile targeted to the desired cell type, was much better tolerated, and the corresponding treated eyes had more cones, a thicker ONL, and an improved ERG cone response versus untreated animals. Degenerated areas were still observed in some RK-treated eyes, but these areas were still directly associated with the injection site. The margins of the degenerated areas, where the effective dose was presumably lower, showed improvement in both cone density and ONL thickness. Surprisingly, IHC analysis showed that the cone density was slightly, but significantly, improved in areas of treated eyes in which RS1 immunoreactivity was not detected. This was true for both CBh- and RK-treated eyes, and supports the hypothesis that low doses, even below the threshold of immunodetectability, could still be cone-protective.

In view of the encouraging results from subretinal delivery, para-retinal administration could be a preferred vector delivery method for treating XLRS. Similar to intravitreal injection and unlike subretinal injection, para-retinal injection does not penetrate the retina and thus is less likely than sub-retinal injection to cause mechanical damages to the retina, which is usually fragile in XLRS cases. On the other hand, para-retina injection is superior than intravitreal injection by localizing the injection bolus immediately adjacent to the retina. Prior studies in NHP models using an AAV204.CBh:GFP vector demonstrated that this technique allows the efficient transduction of all retinal layers. While the mouse eye is too small to achieve para-retinal delivery, the results of subretinal injections in the current mice study nonetheless model efficacy for para-retinal injection in non-human primates (NHP) and human.

### FURTHER NUMBERED EMBODIMENTS

Further numbered embodiments of the present disclosure are provided as follows:

Embodiment 1. A method of treating retinoschisis in a subject in need thereof, comprising para-retinal or sub-retinal administration of an AAV viral vector to the subject.

Embodiment 2. The method of Embodiment 1, wherein the AAV viral vector comprises a photoreceptor-specific promoter operably linked to a transgene encoded by a heterologous nucleic acid.

Embodiment 3. The method of Embodiment 2, wherein the photoreceptor-specific promoter is selected from the group consisting of a rhodopsin kinase (RK) promoter, a rhodopsin (RHO) promoter, a beta phosphodiesterase (PDE) promoter, and a retinitis pigmentosa (RP1) promoter.

Embodiment 4. The method of Embodiment 2, wherein the photoreceptor-specific promoter is a rhodopsin kinase (RK) promoter.

Embodiment 5. The method of Embodiment 4, wherein the RK promoter comprises, consists essentially of, or consists of a nucleic acid sequence having at least 90%, at least 95%, at least 97%, at least 98%, at least 99%, or 100% identity to SEQ ID NO: 196.

Embodiment 6. The method of any one of Embodiments 1-5, comprising para-retinal administration of the AAV viral vector to the subject.

Embodiment 7. The method of any one of Embodiments 1-6, wherein the subject is a human, and wherein the AAV viral vector is administered at a dose of about 1010 to about 1012 viral genome (vg).

Embodiment 8. The method of any one of Embodiments 1-6, wherein the retinoschisis is X-linked retinoschisis.

Embodiment 9. The method of any one of Embodiments 2-8, wherein the transgene is RS1.

Embodiment 10. The method of Embodiment 9, wherein the transgene comprises a nucleic acid sequence having at least 90%, at least 95%, at least 97%, at least 98%, at least 99%, or 100% identity to SEQ ID NO: 117, or wherein the transgene encodes an amino acid sequence having at least 90%, at least 95%, at least 97%, at least 98%, at least 99%, or 100% identity to SEQ ID NO: 143.

Embodiment 11. A method of treating an ocular disease or disorder in a subject in need thereof, comprising administration of an AAV viral vector to the subject, wherein the AAV viral vector comprises an AAV vector genome, wherein the AAV vector genome comprises, in 5′ to 3′ orientation:
(a) a first AAV inverted terminal repeat,
(b) a promoter,
(c) a heterologous nucleic acid encoding Opa1,
(d) a polyadenylation signal, and
(e) a second AAV inverted terminal repeat.

Embodiment 12. The method of Embodiment 11, wherein the promoter is a CBh promoter comprising a sequence having at least 90%, at least 95%, at least 97%, at least 98%, at least 99%, or 100% identity to SEQ ID NO: 154.

Embodiment 13. The method of Embodiment 11, wherein the promoter is a MeCP2 promoter comprising a sequence having at least 90%, at least 95%, at least 97%, at least 98%, at least 99%, or 100% identity to SEQ ID NO: 156.

Embodiment 14. The method of any one of Embodiments 11-13, wherein the AAV vector genome comprises an intron sequence having at least 90%, at least 95%, at least 97%, at least 98%, at least 99%, or 100% identity to SEQ ID NO: 200 or 227, and wherein the intron sequence is located between the promoter and the heterologous nucleic acid encoding Opa1.

Embodiment 15. The method of Embodiment 14, wherein the intron sequence is located immediately downstream of the promoter, without any additional nucleotide in between.

Embodiment 16. The method of any one of Embodiments 11-15, wherein the heterologous nucleic acid encoding Opa1 comprises a sequence having at least 90%, at least 95%, at least 97%, at least 98%, at least 99%, or 100% identity to any one of SEQ ID NO: 175, 182 and 184.

Embodiment 17. The method of any one of Embodiments 11-16, wherein the heterologous nucleic acid encodes an Opa1 protein comprising an amino acid sequence having at least 90%, at least 95%, at least 97%, at least 98%, at least 99%, or 100% identity to any one of SEQ ID NO: 180, 183 and 185.

Embodiment 18. The method of any one of Embodiments 11-16, wherein the heterologous nucleic acid encodes an Opa1 protein comprising an amino acid sequence having at least 90%, at least 95%, at least 97%, at least 98%, at least 99%, or 100% identity to SEQ ID NO: 180.

Embodiment 19. The method of any one of Embodiments 11-18, wherein the polyadenylation signal comprises a sequence having at least 90%, at least 95%, at least 97%, at least 98%, at least 99%, or 100% identity to SEQ ID NO: 201 or 225.

Embodiment 20. The method of any one of Embodiments 11-19, wherein the AAV vector genome does not comprise any telomeric repeats sequence.

Embodiment 21. The method of any one of Embodiments 11-19, wherein the AAV vector genome comprises a first telomeric repeats sequence located between the polyadenylation signal and the second AAV inverted terminal repeat, and wherein the first telomeric repeats sequence has at least 90%, at least 95%, at least 97%, at least 98%, at least 99%, or 100% identity to SEQ ID NO: 202.

Embodiment 22. The method of any one of Embodiments 11-21, wherein the AAV vector genome comprises a second telomeric repeats sequence located between the first AAV inverted terminal repeat and the promoter, and wherein the second telomeric repeats sequence has at least 90%, at least 95%, at least 97%, at least 98%, at least 99%, or 100% identity to SEQ ID NO: 203.

Embodiment 23. The method of any one of Embodiments 11-22, wherein the first AAV inverted terminal repeat comprises or consists of a sequence having at least 90%, at least 95%, at least 97%, at least 98%, at least 99%, or 100% identity to SEQ ID NO: 253, and/or wherein the second AAV inverted terminal repeat comprises or consists of a sequence having at least 90%, at least 95%, at least 97%, at least 98%, at least 99%, or 100% identity to SEQ ID NO: 254.

Embodiment 24. The method of any one of Embodiments 11-23, wherein the AAV vector genome comprises, in 5′ to 3′ orientation:
(a) the first AAV inverted terminal repeat,
(b) the promoter comprising a sequence having at least 90%, at least 95%, at least 97%, at least 98%, at least 99%, or 100% identity to SEQ ID NO: 154,
(c) the heterologous nucleic acid encoding Opa1,
(d) the polyadenylation signal comprises a sequence having at least 90%, at least 95%, at least 97%, at least 98%, at least 99%, or 100% identity to SEQ ID NO: 201, and
(e) the second AAV inverted terminal repeat.

Embodiment 25. The method of Embodiment 24, wherein the AAV vector genome comprises an intron sequence having at least 90%, at least 95%, at least 97%, at least 98%, at least 99%, or 100% identity to SEQ ID NO: 200, and wherein the intron sequence is located between the promoter and the heterologous nucleic acid encoding Opa1.

Embodiment 26. The method of Embodiment 24 or 25, wherein the AAV vector genome comprises a sequence having at least 90%, at least 95%, at least 97%, at least 98%, at least 99%, or 100% identity to SEQ ID NO: 228.

Embodiment 27. The method of any one of Embodiments 24-26, wherein the Opa1 protein comprises an amino acid sequence having at least 90%, at least 95%, at least 97%, at least 98%, at least 99%, or 100% identity to SEQ ID NO: 180.

Embodiment 28. The method of any one of Embodiments 11-27, wherein the AAV vector genome comprises a polynucleotide sequence having at least 90%, at least 95%, at least 97%, at least 98%, at least 99%, or 100% identity to any one of SEQ ID NO: 230-239.

Embodiment 29. The method of any one of Embodiments 11-28, wherein the ocular disease or disorder is Autosomal Dominant Optic Atrophy.

Embodiment 30. A method of treating an ocular disease or disorder in a subject in need thereof, comprising administration of an AAV viral vector to the subject, wherein the AAV viral vector comprises an AAV vector genome, wherein the AAV vector genome comprises, in 5′ to 3′ orientation:
(a) a first AAV inverted terminal repeat,
(b) a promoter,
(c) a heterologous nucleic acid encoding RS1,
(d) a polyadenylation signal, and
(e) a second AAV inverted terminal repeat.

Embodiment 31. The method of Embodiment 30, wherein the promoter is a photoreceptor-specific promoter.

Embodiment 32. The method of Embodiment 31, wherein the photoreceptor-specific promoter is selected from the group consisting of a rhodopsin kinase (RK) promoter, a rhodopsin (Rho) promoter, a beta phosphodiesterase (PDE) promoter, and a retinitis pigmentosa (RP1) promoter.

Embodiment 33. The method of Embodiment 30, wherein the promoter is a CBh promoter comprising a sequence having at least 90%, at least 95%, at least 97%, at least 98%, at least 99%, or 100% identity to SEQ ID NO: 154.

Embodiment 34. The method of Embodiment 30, wherein the promoter is a RK promoter comprising a sequence having at least 90%, at least 95%, at least 97%, at least 98%, at least 99%, or 100% identity to SEQ ID NO: 196.

Embodiment 35. The method of Embodiment 30, wherein the promoter is a Rho promoter comprising a sequence having at least 90%, at least 95%, at least 97%, at least 98%, at least 99%, or 100% identity to SEQ ID NO: 197.

Embodiment 36. The method of Embodiment 30, wherein the promoter is a PDE promoter comprising a sequence having at least 90%, at least 95%, at least 97%, at least 98%, at least 99%, or 100% identity to SEQ ID NO: 198.

Embodiment 37. The method of any one of Embodiments 30-36, wherein the AAV vector genome comprises an IRBP enhancer sequence upstream of the promoter, wherein the IRBP enhancer sequence has at least 90%, at least 95%, at least 97%, at least 98%, at least 99%, or 100% identity to SEQ ID NO: 199.

Embodiment 38. The method of Embodiment 37, wherein the IRBP enhancer sequence is located immediately upstream of the promoter, without any additional nucleotide in between.

Embodiment 39. The method of any one of Embodiments 30-38, wherein the AAV vector genome comprises a CVA-MVM intron sequence having at least 90%, at least 95%, at least 97%, at least 98%, at least 99%, or 100% identity to SEQ ID NO: 226, and wherein the CVA-MVM intron sequence is located between the promoter and the heterologous nucleic acid encoding RS1.

Embodiment 40. The method of any one of Embodiments 30-38, wherein the AAV vector genome comprises an intron sequence having at least 90%, at least 95%, at least 97%, at least 98%, at least 99%, or 100% identity to SEQ ID NO: 200 or 222, and wherein the intron is located between the promoter and the heterologous nucleic acid encoding RS1.

Embodiment 41. The method of any one of Embodiments 30-40, wherein the heterologous nucleic acid encoding RS1 comprises a sequence having at least 90%, at least 95%, at least 97%, at least 98%, at least 99%, or 100% identity to SEQ ID NO: 117.

Embodiment 42. The method of any one of Embodiments 30-41, wherein the heterologous nucleic acid encodes an RS1 protein comprising an amino acid sequence having at least 90%, at least 95%, at least 97%, at least 98%, at least 99%, or 100% identity to SEQ ID NO: 143.

Embodiment 43. The method of any one of Embodiments 30-42, wherein the polyadenylation signal comprises a sequence having at least 90%, at least 95%, at least 97%, at least 98%, at least 99%, or 100% identity to SEQ ID NO: 201 or 225.

Embodiment 44. The method of any one of Embodiments 30-43, wherein the AAV vector genome does not comprise any telomeric repeats sequence.

Embodiment 45. The method of any one of Embodiments 30-43, wherein the AAV vector genome comprises a first telomeric repeats sequence located between the polyadenylation signal and the second AAV inverted terminal repeat, and wherein the first telomeric repeats sequence has at least 90%, at least 95%, at least 97%, at least 98%, at least 99%, or 100% identity to SEQ ID NO: 203.

Embodiment 46. The method of any one of Embodiments 30-45, wherein the AAV vector genome comprises a human beta-globin scaffold/matrix attachment region (βGlo_s/MAR) sequence located between the polyadenylation signal and the second AAV inverted terminal repeat, or the polyadenylation signal and the first telomeric repeats sequence, wherein the βGlo_s/MAR has at least 90%, at least 95%, at least 97%, at least 98%, at least 99%, or 100% identity to SEQ ID NO: 221.

Embodiment 47. The method of any one of Embodiments 30-46, wherein the first AAV inverted terminal repeat comprises or consists of a sequence having at least 90%, at least 95%, at least 97%, at least 98%, at least 99%, or 100% identity to SEQ ID NO: 255, and/or wherein the second AAV inverted terminal repeat comprises or consists of a sequence having at least 90%, at least 95%, at least 97%, at least 98%, at least 99%, or 100% identity to SEQ ID NO: 256.

Embodiment 48. The method of any one of Embodiments 30-47, wherein the AAV vector genome comprises, in 5′ to 3′ orientation:
(a) the first AAV inverted terminal repeat,
(b) the IRBP enhancer sequence having at least 90%, at least 95%, at least 97%, at least 98%, at least 99%, or 100% identity to SEQ ID NO: 199,
(c) the RK promoter comprising a sequence having at least 90%, at least 95%, at least 97%, at least 98%, at least 99%, or 100% identity to SEQ ID NO: 196,
(d) the heterologous nucleic acid encoding RS1,
(e) the polyadenylation signal comprising a sequence having at least 90%, at least 95%, at least 97%, at least 98%, at least 99%, or 100% identity to SEQ ID NO: 225, and
(f) the second AAV inverted terminal repeat.

Embodiment 49. The method of any one of Embodiments 30-48, wherein the AAV vector genome comprises an intron sequence having at least 90%, at least 95%, at least 97%, at least 98%, at least 99%, or 100% identity to SEQ ID NO: 222, and wherein the intron sequence is located between the promoter and the heterologous nucleic acid encoding RS1.

Embodiment 50. The method of Embodiment 49, wherein the AAV vector genome comprises a CBA sequence of SEQ ID NO: 229, or a sequence having at most 5, at most 4, at most 3, at most 2, or at most 1 mutation(s) thereto, and wherein the CBA sequence is located immediately upstream of the intron sequence without any additional nucleotides in between.

Embodiment 51. The method of Embodiment 49 or 50, wherein the AAV vector genome comprises a CBA-MVM intron sequence having at least 90%, at least 95%, at least 97%, at least 98%, at least 99%, or 100% identity to SEQ ID NO: 226, and wherein the CVA-MVM intron sequence is located between the promoter and the heterologous nucleic acid encoding RS1.

Embodiment 52. The method of any one of Embodiments 48-51, wherein the AAV vector genome comprises a sequence having at least 90%, at least 95%, at least 97%, at least 98%, at least 99%, or 100% identity to SEQ ID NO: 224.

Embodiment 53. The method of any one of Embodiments 30-52, wherein the AAV vector genome comprises a polynucleotide sequence having at least 90%, at least 95%, at least 97%, at least 98%, at least 99%, or 100% identity to any one of SEQ ID NO: 224 and 240-252.

Embodiment 54. The method of any one of Embodiments 30-53, wherein the ocular disease or disorder is X-linked retinoschisis.

Embodiment 55. The method of any one of Embodiments 1-54, wherein the AAV viral vector comprises an AAV capsid protein comprising an amino acid sequence that is at least 95% identical to SEQ ID NO: 1-3, 30-34, 49, 67, 84, or 164.

Embodiment 56. The method of Embodiment 55, wherein the AAV viral vector comprises an AAV capsid protein comprising an amino acid sequence that is at least 98%, at least 99%, at least 99.5%, or 100% identical to SEQ ID NO: 1-3, 30-34, 49, 67, 84 or 164.

Embodiment 57. The method of Embodiment 55, wherein the AAV viral vector comprises an AAV capsid protein comprising or consisting of an amino acid sequence that is at least 95%, at least 98%, at least 99%, at least 99.5%, or 100% identical to SEQ ID NO: 2.

Embodiment 58. The method of any one of Embodiments 11-57, wherein the administration is para-retinal administration.

Embodiment 59. The method of any one of Embodiments 1-10 and 58, wherein the para-retinal administration comprises injecting at a distance of between 0 and 13 millimeters (mm), between 0 and 10 mm, between 0 and 5 mm, or between 0 and 3 mm, from the surface of the retina in the posterior vitreous cavity of the eye.

Embodiment 60. The method of any one of Embodiments 1-59, wherein the AAV viral vector is administered at a dose of about 1010 to about 1012 viral genome (vg).

Embodiment 61. The method of any one of Embodiments 1-60, wherein the subject is a human.

Embodiment 62. A nucleic acid comprising, in 5′ to 3′ orientation:
(a) a first AAV inverted terminal repeat,
(b) a promoter,
(c) a heterologous nucleic acid encoding Opa1,
(d) a polyadenylation signal, and
(e) a second AAV inverted terminal repeat.

Embodiment 63. The nucleic acid of Embodiment 62, wherein the promoter is a CBh promoter comprising a sequence having at least 90%, at least 95%, at least 97%, at least 98%, at least 99%, or 100% identity to SEQ ID NO: 154.

Embodiment 64. The nucleic acid of Embodiment 62, wherein the promoter is a MeCP2 promoter comprising a sequence having at least 90%, at least 95%, at least 97%, at least 98%, at least 99%, or 100% identity to SEQ ID NO: 156.

Embodiment 65. The nucleic acid of any one of Embodiments 62-64, wherein the AAV vector genome comprises an intron sequence having at least 90%, at least 95%, at least 97%, at least 98%, at least 99%, or 100% identity to SEQ ID NO: 200 or 227, and wherein the intron sequence is located between the promoter and the heterologous nucleic acid encoding Opa1.

Embodiment 66. The nucleic acid of any one of Embodiments 62-65, wherein the intron sequence is located immediately downstream of the promoter, without any additional nucleotide in between.

Embodiment 67. The nucleic acid of any one of Embodiments 62-66, wherein the heterologous nucleic acid encoding Opa1 comprises a sequence having at least 90%, at least 95%, at least 97%, at least 98%, at least 99%, or 100% identity to any one of SEQ ID NO: 175, 182 and 184.

Embodiment 68. The nucleic acid of any one of Embodiments 62-67, wherein the heterologous nucleic acid encodes an Opa1 protein comprising an amino acid sequence having at least 90%, at least 95%, at least 97%, at least 98%, at least 99%, or 100% identity to any one of SEQ ID NO: 180, 183 and 185.

Embodiment 69. The nucleic acid of any one of Embodiments 62-67, wherein the heterologous nucleic acid encodes an Opa1 protein comprising an amino acid sequence having at least 90%, at least 95%, at least 97%, at least 98%, at least 99%, or 100% identity to SEQ ID NO: 180.

Embodiment 70. The nucleic acid of any one of Embodiments 62-69, wherein the polyadenylation signal comprises a sequence having at least 90%, at least 95%, at least 97%, at least 98%, at least 99%, or 100% identity to SEQ ID NO: 201 or 225.

Embodiment 71. The nucleic acid of any one of Embodiments 62-70, wherein the AAV vector genome does not comprise any telomeric repeats sequence.

Embodiment 72. The nucleic acid of any one of Embodiments 62-70, wherein the AAV vector genome comprises a first telomeric repeats sequence located between the polyadenylation signal and the second AAV inverted terminal repeat, and wherein the first telomeric repeats sequence has at least 90%, at least 95%, at least 97%, at least 98%, at least 99%, or 100% identity to SEQ ID NO: 202.

Embodiment 73. The nucleic acid of any one of Embodiments 62-72, wherein the AAV vector genome comprises a second telomeric repeats sequence located between the first AAV inverted terminal repeat and the promoter, and wherein the second telomeric repeats sequence has at least 90%, at least 95%, at least 97%, at least 98%, at least 99%, or 100% identity to SEQ ID NO: 203.

Embodiment 74. The nucleic acid of any one of Embodiments 62-73, wherein the first AAV inverted terminal repeat comprises or consists of a sequence having at least 90%, at least 95%, at least 97%, at least 98%, at least 99%, or 100% identity to SEQ ID NO: 253, and/or wherein the second AAV inverted terminal repeat comprises or consists of a sequence having at least 90%, at least 95%, at least 97%, at least 98%, at least 99%, or 100% identity to SEQ ID NO: 254.

Embodiment 75. The nucleic acid of any one of Embodiments 62-74, wherein the AAV vector genome comprises, in 5′ to 3′ orientation:
(a) the first AAV inverted terminal repeat,
(b) the promoter comprising a sequence having at least 90%, at least 95%, at least 97%, at least 98%, at least 99%, or 100% identity to SEQ ID NO: 154,
(c) the heterologous nucleic acid encoding Opa1,
(d) the polyadenylation signal comprises a sequence having at least 90%, at least 95%, at least 97%, at least 98%, at least 99%, or 100% identity to SEQ ID NO: 201, and
(e) the second AAV inverted terminal repeat.

Embodiment 76. The nucleic acid of Embodiment 75, comprising an intron sequence having at least 90%, at least 95%, at least 97%, at least 98%, at least 99%, or 100% identity to SEQ ID NO: 200, and wherein the intron sequence is located between the promoter and the heterologous nucleic acid encoding Opa1.

Embodiment 77. The nucleic acid of Embodiment 75 or 76, wherein the Opa1 protein comprises an amino acid sequence having at least 90%, at least 95%, at least 97%, at least 98%, at least 99%, or 100% identity to SEQ ID NO: 180.

Embodiment 78. The nucleic acid of any one of Embodiments 75-77, comprising a sequence having at least 90%, at least 95%, at least 97%, at least 98%, at least 99%, or 100% identity to SEQ ID NO: 228.

Embodiment 79. The nucleic acid of any one of Embodiments 62-78, comprising a polynucleotide sequence having at least 90%, at least 95%, at least 97%, at least 98%, at least 99%, or 100% identity to any one of SEQ ID NO: 230-239.

Embodiment 80. A nucleic acid comprising, in 5′ to 3′ orientation:
(a) a first AAV inverted terminal repeat,
(b) a promoter,
(c) a heterologous nucleic acid encoding RS1,
(d) a polyadenylation signal, and
(e) a second AAV inverted terminal repeat.

Embodiment 81. The nucleic acid of Embodiment 80, wherein the promoter is a photoreceptor-specific promoter.

Embodiment 82. The nucleic acid of Embodiment 81, wherein the photoreceptor-specific promoter is selected from the group consisting of a rhodopsin kinase (RK) promoter, a rhodopsin (RHO) promoter, a beta phosphodiesterase (PDE) promoter, and a retinitis pigmentosa (RP1) promoter.

Embodiment 83. The nucleic acid of Embodiment 80, wherein the promoter is a CBh promoter comprising a sequence having at least 90%, at least 95%, at least 97%, at least 98%, at least 99%, or 100% identity to SEQ ID NO: 154.

Embodiment 84. The nucleic acid of Embodiment 80, wherein the promoter is a RK promoter comprising a sequence having at least 90%, at least 95%, at least 97%, at least 98%, at least 99%, or 100% identity to SEQ ID NO: 196.

Embodiment 85. The nucleic acid of Embodiment 80, wherein the promoter is a Rho promoter comprising a sequence having at least 90%, at least 95%, at least 97%, at least 98%, at least 99%, or 100% identity to SEQ ID NO: 197.

Embodiment 86. The nucleic acid of Embodiment 80, wherein the promoter is a PDE promoter comprising a sequence having at least 90%, at least 95%, at least 97%, at least 98%, at least 99%, or 100% identity to SEQ ID NO: 198.

Embodiment 87. The nucleic acid of any one of Embodiments 80-86, comprising an IRBP enhancer sequence upstream of the promoter, wherein the IRBP enhancer sequence has at least 90%, at least 95%, at least 97%, at least 98%, at least 99%, or 100% identity to SEQ ID NO: 199.

Embodiment 88. The nucleic acid of Embodiments 87, wherein the IRBP enhancer sequence is located immediately upstream of the promoter, without any additional nucleotide in between.

Embodiment 89. The nucleic acid of any one of Embodiments 80-88, wherein the AAV vector genome comprises a CVA-MVM intron sequence having at least 90%, at least 95%, at least 97%, at least 98%, at least 99%, or 100% identity to SEQ ID NO: 226, and wherein the CVA-MVM intron sequence is located between the promoter and the heterologous nucleic acid encoding RS1.

Embodiment 90. The nucleic acid of any one of Embodiments 80-88, wherein the AAV vector genome comprises an intron sequence having at least 90%, at least 95%, at least 97%, at least 98%, at least 99%, or 100% identity to SEQ ID NO: 200 or 222, and wherein the intron is located between the promoter and the heterologous nucleic acid encoding RS1.

Embodiment 91. The nucleic acid of any one of Embodiments 80-88, wherein the heterologous nucleic acid encoding RS1 comprises a sequence having at least 90%, at least 95%, at least 97%, at least 98%, at least 99%, or 100% identity to SEQ ID NO: 117.

Embodiment 92. The nucleic acid of any one of Embodiments 80-91, wherein the heterologous nucleic acid encodes an RS1 protein comprising an amino acid sequence having at least 90%, at least 95%, at least 97%, at least 98%, at least 99%, or 100% identity to SEQ ID NO: 143.

Embodiment 93. The nucleic acid of any one of Embodiments 80-92, wherein the polyadenylation signal comprises a sequence having at least 90%, at least 95%, at least 97%, at least 98%, at least 99%, or 100% identity to SEQ ID NO: 201 or 225.

Embodiment 94. The nucleic acid of any one of Embodiments 80-93, wherein the AAV vector genome does not comprise any telomeric repeats sequence.

Embodiment 95. The nucleic acid of any one of Embodiments 80-93, wherein the AAV vector genome comprises a first telomeric repeats sequence located between the polyadenylation signal and the second AAV inverted terminal repeat, and wherein the first telomeric repeats sequence has at least 90%, at least 95%, at least 97%, at least 98%, at least 99%, or 100% identity to SEQ ID NO: 203.

Embodiment 96. The nucleic acid of any one of Embodiments 80-95, wherein the AAV vector genome comprises a human beta-globin scaffold/matrix attachment region (βGlo_s/MAR) sequence located between the polyadenylation signal and the second AAV inverted terminal repeat, or the polyadenylation signal and the first telomeric repeats sequence, wherein the βGlo_s/MAR sequence has at least 90%, at least 95%, at least 97%, at least 98%, at least 99%, or 100% identity to SEQ ID NO: 221.

Embodiment 97. The nucleic acid of any one of Embodiments 80-96, wherein the first AAV inverted terminal repeat comprises or consists of a sequence having at least 90%, at least 95%, at least 97%, at least 98%, at least 99%, or 100% identity to SEQ ID NO: 255, and/or wherein the second AAV inverted terminal repeat comprises or consists of a sequence having at least 90%, at least 95%, at least 97%, at least 98%, at least 99%, or 100% identity to SEQ ID NO: 256.

Embodiment 98. The nucleic acid of any one of Embodiments 80-97, wherein the AAV vector genome comprises, in 5′ to 3′ orientation:
(a) the first AAV inverted terminal repeat,
(b) the IRBP enhancer sequence having at least 90%, at least 95%, at least 97%, at least 98%, at least 99%, or 100% identity to SEQ ID NO: 199,
(c) the RK promoter comprising a sequence having at least 90%, at least 95%, at least 97%, at least 98%, at least 99%, or 100% identity to SEQ ID NO: 196,
(d) the heterologous nucleic acid encoding RS1,
(e) the polyadenylation signal comprising a sequence having at least 90%, at least 95%, at least 97%, at least 98%, at least 99%, or 100% identity to SEQ ID NO: 225, and
(f) the second AAV inverted terminal repeat.

Embodiment 99. The nucleic acid of any one of Embodiments 80-98, comprising an intron sequence having at least 90%, at least 95%, at least 97%, at least 98%, at least 99%, or 100% identity to SEQ ID NO: 222, and wherein the intron sequence is located between the promoter and the heterologous nucleic acid encoding RS1.

Embodiment 100. The nucleic acid of Embodiment 99, comprising a CBA sequence of SEQ ID NO: 229, or a sequence having at most 5, at most 4, at most 3, at most 2, or at most 1 mutation(s) thereto, and wherein the CBA sequence is located immediately upstream of the intron sequence without any additional nucleotides in between.

Embodiment 101. The nucleic acid of Embodiment 99 or 100, comprising a CBA-MVM intron sequence having at least 90%, at least 95%, at least 97%, at least 98%, at least 99%, or 100% identity to SEQ ID NO: 226, and wherein the CVA-MVM intron sequence is located between the promoter and the heterologous nucleic acid encoding RS1.

Embodiment 102. The nucleic acid of any one of Embodiments 98-101, comprising a sequence having at least 90%, at least 95%, at least 97%, at least 98%, at least 99%, or 100% identity to SEQ ID NO: 224.

Embodiment 103. The nucleic acid of any one of Embodiments 80-102, comprising a polynucleotide sequence having at least 90%, at least 95%, at least 97%, at least 98%, at least 99%, or 100% identity to any one of SEQ ID NO: 224 and 240-252.

Embodiment 104. A nucleic acid comprising, in 5′ to 3′ orientation:
(a) a promoter,
(b) a heterologous nucleic acid encoding a transgene, and
(c) a polyadenylation signal,
   wherein the promoter is a CBh promoter comprising a sequence having at least 90%, at least 95%, at least 97%, at least 98%, at least 99%, or 100% identity to SEQ ID NO: 154, and wherein the polyadenylation signal comprises a sequence having at least 90%, at least 95%, at least 97%, at least 98%, at least 99%, or 100% identity to SEQ ID NO: 201 or 225.

Embodiment 105. The nucleic acid of Embodiment 104, comprising an intron sequence having at least 90%, at least 95%, at least 97%, at least 98%, at least 99%, or 100% identity to SEQ ID NO: 200, 222, 226, or 227, and wherein the intron is located between the promoter and the heterologous nucleic acid encoding the transgene.

Embodiment 106. The nucleic acid of any one of Embodiments 104-105, comprising a first ITR that is located 5′ to the promoter, and a second ITR that is located 3′ to the polyadenylation signal.

Embodiment 107. The nucleic acid of any one of Embodiments 104-106, wherein the nucleic acid does not comprise any telomeric repeats sequence.

Embodiment 108. The nucleic acid of any one of Embodiments 104-106, comprising a first telomeric repeats sequence located between the polyadenylation signal and the second AAV ITR, and wherein the first telomeric repeats sequence has at least 90%, at least 95%, at least 97%, at least 98%, at least 99%, or 100% identity to SEQ ID NO: 202.

Embodiment 109. The nucleic acid of any one of Embodiments 104-108, comprising a second telomeric repeats sequence located between the first AAV inverted terminal repeat and the promoter, and wherein the second telomeric repeats sequence has at least 90%, at least 95%, at least 97%, at least 98%, at least 99%, or 100% identity to SEQ ID NO: 203.

Embodiment 110. A vector comprising the nucleic acid of any one of Embodiments 62-109.

Embodiment 111. An AAV vector genome comprising the nucleic acid of any one of Embodiments 62-109.

Embodiment 112. An AAV viral vector comprising the AAV vector genome of Embodiment 111.

Embodiment 113. The AAV viral vector of Embodiment 112, wherein the AAV viral vector comprises the AAV capsid protein comprising an amino acid sequence that is at least 95%, at least 98%, at least 99%, at least 99.5%, or 100% identical to any one of SEQ ID NO: 1-3, 30-34, 49, 84 and 164.

Embodiment 114. A method of expressing a transgene in a retinal cell, comprising delivering the nucleic acid of any one of Embodiments 62-109 to the retinal cell, or transducing the retinal cell with the AAV viral vector of any one of Embodiments 112-113.

Embodiment 115. The method of Embodiment 114, wherein the retinal cell is a retinal ganglion cell.

Embodiment 116. A method of treating a disease or disorder comprising administering the AAV viral vector of any one of Embodiments 112-113 to a subject.

Embodiment 117. The method of Embodiment 116, wherein the AAV viral vector is administered to the subject intra-ocularly, peri-ocularly, intravitreally, para-retinally, or sub-retinally.

Embodiment 118. The method of Embodiment 116 or 117, wherein the disease or disorder is macular degeneration, retinitis pigmentosa, Autosomal Dominant Optic Atrophy, Retinoschisis, Stargardt disease, Bietti's Crystalline Dystrophy or BEST vitelliform macular dystrophy.

Embodiment 119. The method of Embodiment 116 or 117, wherein the disease or disorder is X-linked Retinoschisis (XLRS).

1.-119. (canceled)

120. A nucleic acid comprising, in 5′ to 3′ orientation:
(a) a first AAV inverted terminal repeat,
(b) a promoter,
(c) a heterologous nucleic acid encoding Retinoschisin 1 (RS1),
(d) a polyadenylation signal, and
(e) a second AAV inverted terminal repeat,
wherein the nucleic acid comprises an IRBP enhancer sequence upstream of the promoter, wherein the IRBP enhancer sequence has at least 90%, at least 95%, at least 97%, at least 98%, at least 99%, or 100% identity to SEQ ID NO: 199.

121. The nucleic acid of claim 120, wherein the IRBP enhancer sequence is located immediately upstream of the promoter, without any additional nucleotide in between.

122. The nucleic acid of claim 120, wherein the promoter is a RK promoter comprising a sequence having at least 90%, at least 95%, at least 97%, at least 98%, at least 99%, or 100% identity to SEQ ID NO: 196.

123. The nucleic acid of claim 120, wherein the nucleic acid comprises a CVA-MVM intron sequence having at least 90%, at least 95%, at least 97%, at least 98%, at least 99%, or 100% identity to SEQ ID NO: 226, and wherein the CVA-MVM intron sequence is located between the promoter and the heterologous nucleic acid encoding RS1.

124. The nucleic acid of claim 120, wherein the heterologous nucleic acid encoding RS1 comprises a sequence having at least 90%, at least 95%, at least 97%, at least 98%, at least 99%, or 100% identity to SEQ ID NO: 117.

125. The nucleic acid of claim 124, wherein the heterologous nucleic acid encodes an RS1 protein comprising an amino acid sequence having at least 90%, at least 95%, at least 97%, at least 98%, at least 99%, or 100% identity to SEQ ID NO: 143.

126. The nucleic acid of claim 120, wherein the polyadenylation signal comprises a sequence having at least 90%, at least 95%, at least 97%, at least 98%, at least 99%, or 100% identity to SEQ ID NO: 225.

127. The nucleic acid of claim 120, wherein the first AAV inverted terminal repeat comprises or consists of a sequence having at least 90%, at least 95%, at least 97%, at least 98%, at least 99%, or 100% identity to SEQ ID NO: 255, and/or wherein the second AAV inverted terminal repeat comprises or consists of a sequence having at least 90%, at least 95%, at least 97%, at least 98%, at least 99%, or 100% identity to SEQ ID NO: 256.

128. The nucleic acid of claim 120, wherein the nucleic acid comprises, in 5′ to 3′ orientation:
(a) the first AAV inverted terminal repeat,
(b) the IRBP enhancer sequence having at least 90%, at least 95%, at least 97%, at least 98%, at least 99%, or 100% identity to SEQ ID NO: 199,
(c) an RK promoter comprising a sequence having at least 90%, at least 95%, at least 97%, at least 98%, at least 99%, or 100% identity to SEQ ID NO: 196,
(d) the heterologous nucleic acid encoding RS1,
(e) the polyadenylation signal comprising a sequence having at least 90%, at least 95%, at least 97%, at least 98%, at least 99%, or 100% identity to SEQ ID NO: 225, and
(f) the second AAV inverted terminal repeat.

129. The nucleic acid of claim 128, comprising a sequence having 100% identity to SEQ ID NO: 224.

130. A vector or an AAV vector genome comprising the nucleic acid of claim 120.

131. An AAV viral vector comprising the AAV vector genome of claim 130.

132. The AAV viral vector of claim 131, wherein the AAV viral vector comprises an AAV capsid protein comprising an amino acid sequence that is at least 95%, at least 98%, at least 99%, at least 99.5%, or 100% identical to SEQ ID NO: 2.

133. A method of expressing Retinoschisin 1 (RS1) in a retinal cell, comprising transducing the retinal cell with the AAV viral vector of claim 131.

134. A method of treating a disease or disorder in a subject in need thereof, comprising administering the AAV viral vector of claim 131 to the subject.

135. The method of claim 134, wherein the AAV viral vector is administered to the subject para-retinally, or sub-retinally.

136. The method of claim 135, wherein the AAV viral vector is administered at a dose of about 10¹⁰ to about 10¹² viral genome (vg).

137. The method of claim 134, wherein the disease or disorder is X-linked Retinoschisis (XLRS).

138. A nucleic acid comprising, in 5′ to 3′ orientation:
(a) a first AAV inverted terminal repeat,
(b) a promoter,
(c) a heterologous nucleic acid encoding Opa1,
(d) a polyadenylation signal, and
(e) a second AAV inverted terminal repeat.

139. A method of treating a disease or disorder in a subject in need thereof, comprising administering a vector comprising the nucleic acid of claim 138 to the subject.
